(19) United States
(12) Patent Application Publication
(10) Pub. No.: US 2008/0182783 A1
(43) Pub. Date: Jul. 31, 2008

(54) Growth Hormone Conjugates

(75) Inventors: Florencio Zaragoza Dörwald, Smorum (DK); Lars Fogh Iversen, Holte (DK); Nils Langeland Johansen, Copenhagen O (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsvaerd (DK)

(21) Appl. No.: 11/577,238

(22) PCT Filed: Oct. 18, 2005
(86) PCT No.: PCT/EP05/55336

§ 371 Date: Nov. 19, 2007

Related U.S. Application Data
(60) Provisional application No. 60/621,207, filed on Oct. 22, 2004.
(60) Provisional application No. 60/647,605, filed on Jan. 27, 2005.

(30) Foreign Application Priority Data
Oct. 18, 2004 (DK) PA 2004 01588
Jan. 21, 2005 (DK) PA 2005 00107

(51) Int. Cl. C07K 14/61; C12N 15/63; C12N 15/16; A61K 38/27

(52) U.S. Cl. 514/12; 530/399; 536/23.51; 435/320.1

(57) ABSTRACT

Compounds of formula (I)

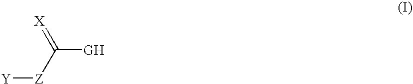

wherein GH represent a radical derived from a growth hormone compound by removal of one hydrogen atom from the N-terminal amino group; X represents oxygen or two hydrogen atoms; Z represents a bond, alkylene, arylene, heteroarylene, —CH₂—O—(CH₂)₁₋₁₀—, —CH₂—O—(C₆H₄)—, or combinations thereof; and
Y represents a radical selected from

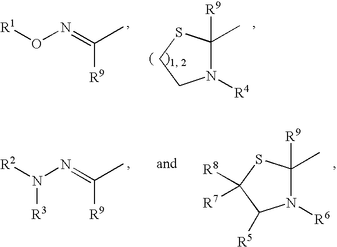

are provided together with methods for making said compounds. The compounds are useful in therapy.

### FIELD OF THE INVENTION

The present invention relates to conjugates of growth hormones with improved pharmacological properties, and to the use of said hormones in therapy.

### BACKGROUND OF THE INVENTION

It is well-known to modify the properties and characteristics of proteins by conjugating groups to said proteins which duly change the properties. Such conjugation generally requires some functional group in the protein to react with another functional group in a conjugating group. Typically, amino groups, such as the N-terminal amino group, the C-amino group in lysines or the cysteine thiol group have been used in combination with a suitable acylating reagent.

It is often desired or even required to conjugate at specific site(s), and this is referred to as regioselective conjugation. Regioselective acylation of growth hormones, such as e.g. human growth hormone (hGH) is, however, difficult, because this protein contains nine lysine residues of similar reactivity, and mixtures of products usually results. The single components of these mixtures are difficult to isolate, and will usually be obtained in low yield and purity only.

Gaertner et al in Bioconjugate Chem., 7, 38-44, 1996 disclose a method for conjugating PEG to IL-8, G-CFS and IKL-1ra by generating an aldehyde at the N-terminus, followed by reaction with an alkoxyamine functionalised PEG. The aldehyde is generated at the N-terminus either by oxidation with periodate if the N-terminal amino acid residue is serine, or by metal catalysed transamination if the N-terminal amino acid residue is different from serine.

N-terminal serine extended human growth hormone, Ser-hGH, is disclosed as SEQ ID No. 66 in WO 04/007687. This application relates to multimeric forms of e.g. hGH with improved properties.

It is also known that human growth hormone may be reductively alkylated with aldehydes selectively at the N-terminal amino group (US 20040127417).

### SUMMARY OF THE INVENTION

The present invention relates to growth hormone compounds (GH) selectively conjugated at the N-terminal to improve pharmacological properties compared to the parent growth hormone compound.

Accordingly, in one embodiment the invention provides compounds of formula (I)

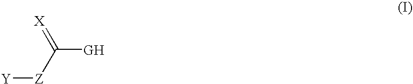

wherein GH represent a radical derived from a growth hormone compound by removal of one hydrogen atom from the N-terminal amino group;
X represents oxygen or two hydrogen atoms;
Z represents a bond, alkylene, arylene, heteroarylene, —CH₂—O—(CH₂)₁₋₁₀—, —CH₂—O—(C₆H₄)—, or combinations thereof;
Y represents a radical selected from

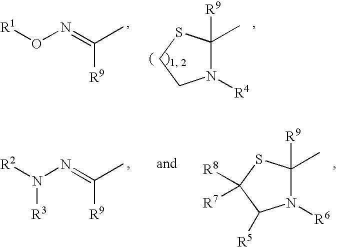

wherein
R¹ and R⁴ independently represent MeO—(CH₂CH₂O)₁₀₀₋₁₀₀₀-E-, [MeO—(CH₂CH₂O)₁₀₀₋₁₀₀₀]—C(═O)NH—(CH₂)₄CH[MeO—(CH₂CH₂O)₁₀₀₋₁₀₀₀C(═O)NH]E-, Me-(CH₂)₁₀₋₃₀-E-, Me-(CH₂)₁₀₋₃₀—C(═O)—NH—S(═O)₂—(CH₂)₂₋₁₀-E-, (5-tetrazolyl)-(CH₂)₁₀₋₃₀-E-, (5-tetrazolyl)₂CH—(CH₂)₁₀₋₃₀-E-, (5-tetrazolyl)-(CH₂)₁₋₃₀—C(═O)—NH—S(═O)₂—(CH₂)₂₋₁₀-E-, (5-tetrazoly)-(C₆H₄)—O—(CH₂)₁₋₃₀, (5-tetrazoly)-(C₆H₄)—(C₆H₄)—O—(CH₂)₁₋₃₀—, HO₂C—(CH₂)₁₋₃₀-E-, cibacronyl-E-, GH-C(═O)—CH═N—O—(CH₂)₂₃₀—, GH-C(═O)—CH═N—(OCH₂CH₂)₁₋₁₀—, GH-(C═O)—CH═N—NH—(CH₂)₂₋₃₀—, GH-(C═O)—CH═N—NH—(CH₂CH₂O)₁₋₃₀—(CH₂)₀₋₃₀—,

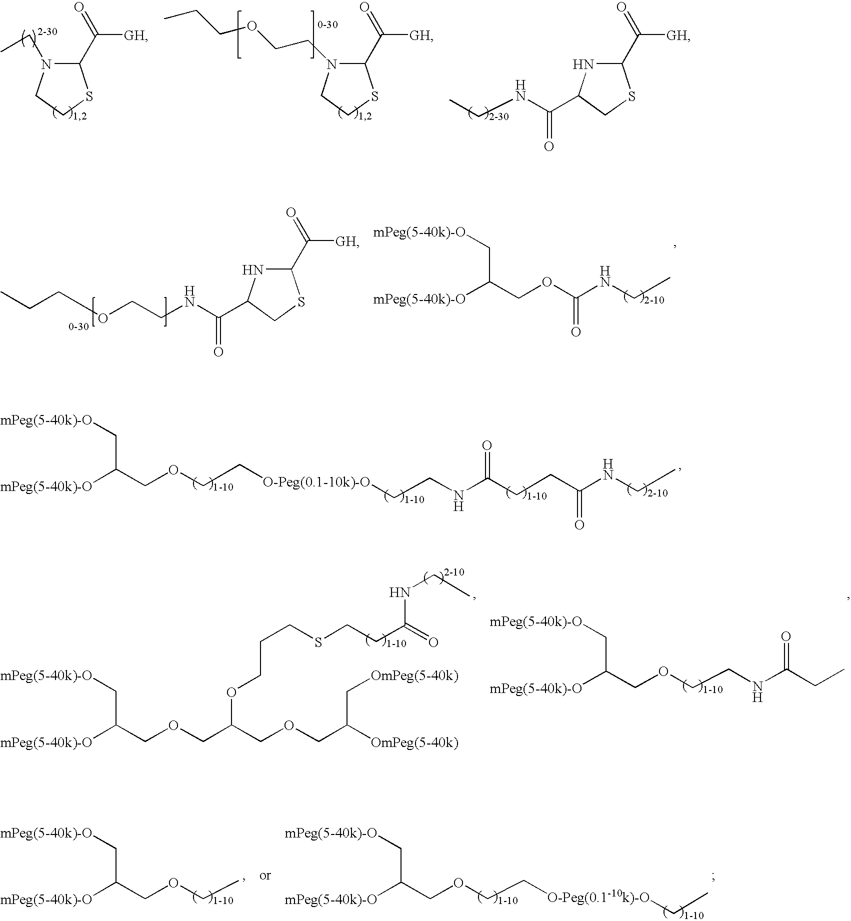

R², R³, R⁵ and R⁶ independently represent

H, C₁₋₆alkyl-, MeO—(CH₂CH₂O)₁₋₁₀₀₀-E-, [MeO—(CH₂CH₂O)₁₋₁₀₀₀]—C(═O)NH—(CH₂)₄CH[MeO—(CH₂CH₂O)₁₋₁₀₀₀—C(═O)NH]E-, Me-(CH₂)₁₋₃₀-E-, Me-(CH₂)₁₀₋₃₀—C(═O)—NH—S(═O)₂—(CH₂)₂₋₁₀-E-, (5-tetrazolyl)-(CH₂)₁₀₋₃₀-E-, (5-tetrazolyl)₂CH—(CH₂)₁₀₋₃₀-E-, (5-tetrazolyl)-(CH₂)₁₋₃₀—C(═O)—NH—S(═O)₂—(CH₂)₂₋₁₀-E-, HO₂C—(CH₂)₁₀₋₃₀-E-, cibacronyl-E-, GH-C(═O)—CH═N—O—(CH₂)₂₋₃₀—, GH-C(═O)—CH═N—O—(OCH₂CH₂)₁₋₁₀—, GH-(C═O)—CH═N—NH—(CH₂)₂₃₀—, GH-(C═O)—CH═N—NH—(CH₂CH₂O)₁₋₃₀—(CH₂)₀₋₃₀—,

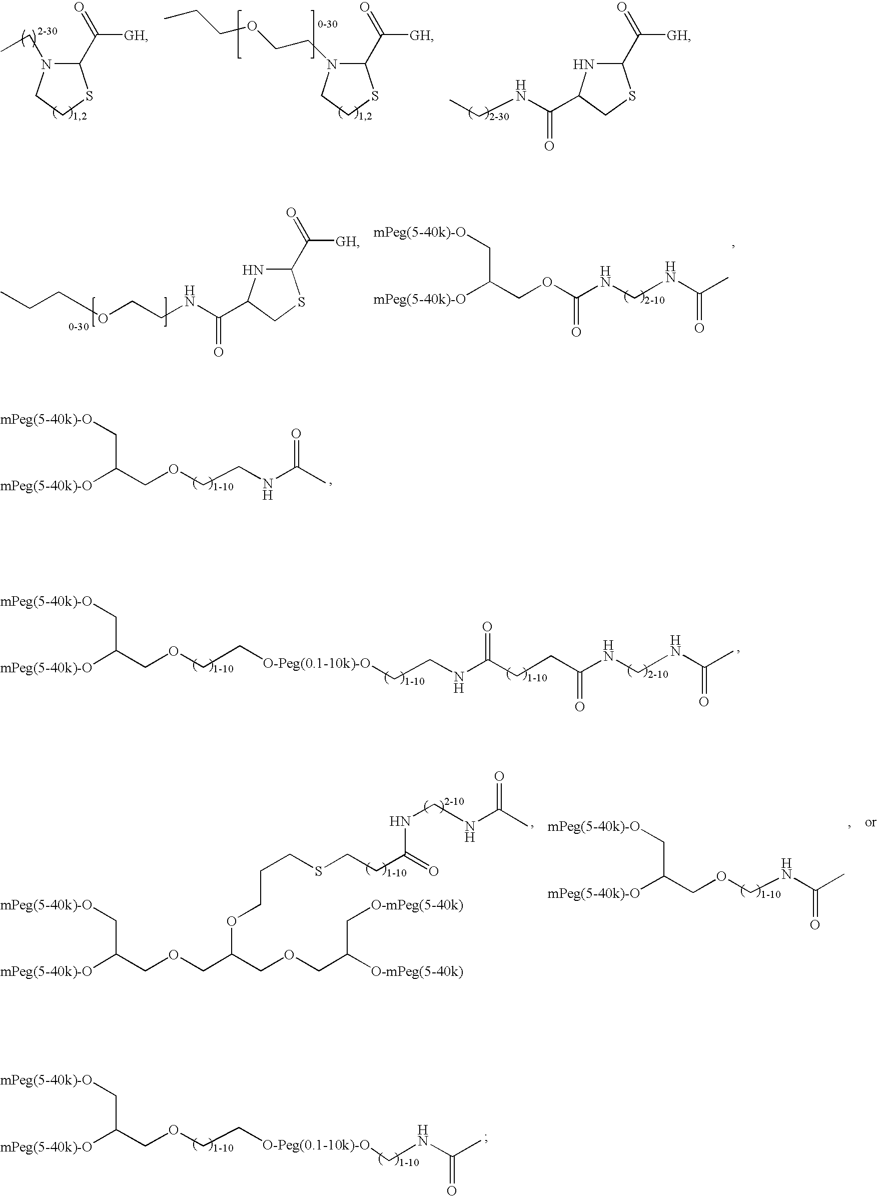

provided that at least one of R² and R³, or at least one of R⁵ and R⁶, does not represent H or C₁₋₆alkyl;
E represents a bond or a diradical selected from
—C(═O)NH(CH₂)₂₋₃₀—, —(CH₂)₁₋₃₀C(═O)NH(CH₂)₂₋₃₀—, —(CH₂)₀₋₃₀C(═O)NH—(CH₂CH₂O)₁₋₁₀—(CH₂)₁₋₅—C(═O)—, —C(═O)NH—[(CH₂CH₂O)₁₋₁₀—(CH₂)₁₋₅—C(═O)]₁₋₅NH(CH₂)₂₋₃₀—, —C(═O)—, —NHC(═O)—, —C(═O)NH—, —(CH₂)₁₋₃₀NHC(═O)—, —(CH₂)₁₋₃₀C(═O)—, —NHC(═O)NH(CH₂)₂₋₃₀—, —(CH₂)₁₋₃₀NHC(═O)NH(CH₂)₂₋₃₀O—,

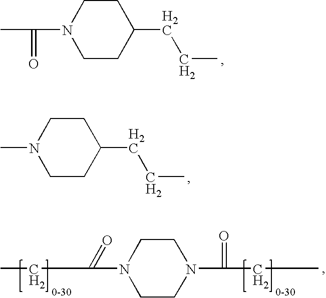

—(CH₂)₀₋₃₀C(═O)NH(CH₂)₂₋₃₀—NHC(═O)—(CH₂)₀₋₃₀—, —(CH₂)₀₋₃₀C(═O)NH(CH₂CH₂O)₁₋₃₀—CH₂CH₂NHC(═O)—(CH₂)₀₋₃₀—, —NH(CH₂)₂₋₃₀—, —NH—, —O—, —S—;
R⁷, R⁸ and R⁹ independently represent H, C₁₋₆alkyl, aryl, or heteroaryl;
and pharmaceutically acceptable salts, prodrugs and solvates thereof.

In one embodiment, the invention relates to methods of preparing compounds of formula I, said method comprising the steps of

(a) reductive alkylation of the N-terminal amino group in GH with a compound of the formula Q-Z-CH═O, wherein Q is a functional group which can be converted into an aldehyde or a ketone by treatment with a suitable reagent to obtain a compound of formula II

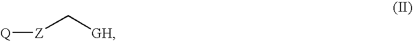

(b) conversion of the functional group Q in the compound of formula (II) into the aldehyde or ketone of formula (III′)

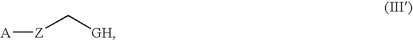

wherein A represents an aldehyde or a ketone moiety; and
(c) condensation of the compound of formula (III′) with a compound selected from R¹—O—NH₂, R²R³N—NH₂, R⁴NH(CH₂)₂₋₃SH or R⁵CH(NR⁶H)(CR⁷R⁸)₁₋₂SH.

In one embodiment, the invention relates to methods of preparing compounds according to formula (I), said method comprising the steps of

(a′) oxidation of the N-terminal serine extended GH of formula (IV)

Ser-GH (IV)

to a glyoxylic acid derivative of formula (V)

O═CH—C(═O)-GH (V); and

(b′) condensation of said derivative of formula V with a compound selected from R¹—O—NH₂, R²R³N—NH₂, R⁴NH(CH₂)₂₋₃SH or R⁵CH(NR⁶H)(CR⁷R⁸)₁₋₂SH.

In one embodiment, the invention relates to a compound of formula (II) or (III)

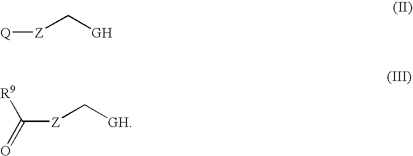

In one embodiment, the invention provides a compound according to formula IV or V,

Ser-GH (IV)

O═CH—C(═O)-GH (V),

provided said compound is not Ser-hGH.

In one embodiment, the invention provides the use of a compound according to formula (II) or formula (III) in the preparation of conjugated GH with improved pharmacological properties compared to the parent GH.

In one embodiment, the invention provides the use of a compound according to formula (IV) or formula (V) in the preparation of conjugated GH with improved pharmacological properties compared to the parent GH.

In one embodiment, the invention relates to a nucleic acid construct comprising a nucleic acid sequence encoding a compound according to formula V, provided that said construct does not encode Ser-hGH, to a vector comprising said construct, and to a host cell comprising said vector.

In one embodiment, the invention provides compounds according to formula (I) for use in therapy.

In one embodiment, the invention provides a pharmaceutical composition comprising a compound according formula (I).

In one embodiment, the invention provides a method of treating diseases benefiting from an increase in the level of circulating growth hormone, the method comprising the administration of a therapeutically effective amount of a compound according to formula (I) to a patient in need thereof.

In one embodiment, the invention relates to the use of a compound according to formula (I) in the manufacture of a medicament for the treatment of a disease benefiting from an increase in the level of circulating growth hormone.

### DEFINITIONS

In the present context, “growth hormone” (GH) is intended to indicate a protein which exhibits growth hormone activity as determined in assay I herein. A protein which exhibits an activity above 20%, such as above 40%, such as above 60%, such as above 80% of that of hGH in said assay is defined as a growth hormone compound.

In the present context, a protein is intended to indicate a sequence of two or more amino acids joined by peptide bonds, wherein said amino acids may be natural or un-natural. It is to be understood that the term is also intended to include proteins which have been derivatized, e.g. by the attachment of lipophilic groups, PEG or prosthetic groups.

In the present context, the term “alkyl” is intended to indicate a straight, branched and/or cyclic saturated monovalent hydrocarbon radical having from one to six carbon atoms, also denoted as C₁₋₆-alkyl. C₁₋₆-alkyl groups include e.g. methyl, ethyl, n-propyl, isopropyl, n-butyl, sec-butyl, isobutyl, tert-butyl, n-pentyl, 2-methylbutyl, 3-methylbutyl, 4-methylpentyl, n-hexyl, 1,1-dimethylpropyl, 1,2-dimethylpropyl, 2,2-dimethylpropyl(neopentyl) and 1,2,2-trimethylpropyl. The term “alkylene” indicates the corresponding bi-radical.

The term “aryl” as used herein is intended to indicate carbocyclic aromatic ring systems comprising one or more rings, such as phenyl, biphenylyl, naphthyl, anthracenyl, phenanthrenyl, fluorenyl, indenyl, pentalenyl and azulenyl. Aryl is also intended to include the partially hydrogenated derivatives of the multi-ring carbocyclic systems enumerated above, wherein at least one ring is aromatic. Examples of such partially hydrogenated derivatives include 1,2,3,4-tetrahydronaphthyl and 1,4-dihydronaphthyl. The term “arylene” is intended to indicate the corresponding bi-radical, and examples include 1,2-phenylene, 1,3-phenylene, 1,4-phenylene, 1,2-naphthylene, 1,4-naphthylene, 4,4′-biphenylene, 4,4″-terphenylene and 4,4′″-quaterphenylene.

The term “heteroaryl” as used herein is intended to indicate radicals of heterocyclic aromatic ring systems containing one or more heteroatoms selected from nitrogen, oxygen and sulphur, such as furyl, thienyl, pyrrolyl, oxazolyl, thiazolyl, imidazolyl, isoxazolyl, isothiazolyl, 1,2,3-triazolyl, 1,2,4-triazolyl, pyranyl, pyridyl, pyridazinyl, pyrimidinyl, pyrazinyl, 1,2,3-triazinyl, 1,2,4-triazinyl, 1,3,5-triazinyl, 1,2,3-oxadiazolyl, 1,2,4-oxadiazolyl, 1,2,5-oxadiazolyl, 1,3,4-oxadiazolyl, 1,2,3-thiadiazolyl, 1,2,4-thiadiazolyl, 1,2,5-thiadiazolyl, 1,3,4-thiadiazolyl, tetrazolyl, thiadiazinyl, indolyl, isoindolyl, benzofuryl, benzothienyl, indazolyl, benzimidazolyl, benzthiazolyl, benzisothiazolyl, benzoxazolyl, benzisoxazolyl, purinyl, quinazolinyl, quinolizinyl, quinolinyl, isoquinolinyl, quinoxalinyl, naphthyridinyl, pteridinyl, carbazolyl, azepinyl, diazepinyl, acridinyl and the like. The term is also intended to include partially hydrogenated derivatives of the multi-ring heterocyclic systems enumerated above, provided at least one ring comprising a hetero atom is aromatic. Examples of such partially hydrogenated derivatives include 2,3-dihydrobenzofuranyl, pyrrolinyl, pyrazolinyl, indolinyl, oxazolidinyl, oxazolinyl and oxazepinyl. The term “heteroarylene” is intended to indicate the corresponding bi-radical, and examples include 1,2,4-pyrazol-2,5-diyl, imidazol-1,2-diyl, thiazol-2,4-diyl, (4-phenylimidazole)-4,1′-diyl and (3,5-diphenyl-1,2,4-oxadiazole)-4,4″-diyl.

The term “cibacronyl” means the radical sketched below, or any salt or solvate of the same:

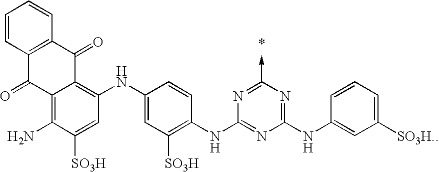

As used herein, the term “solvate” is a complex of defined stoichiometry formed by a solute and a solvent. Solvents may be, by way of example, water, ethanol, or acetic acid.

The term “dipolar solvent” refers to a solvent with a dielectric constant larger than 6.0.

The term “Peg” means a polydisperse or monodisperse diradical of the structure

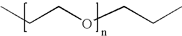

wherein n is an integer larger than 1, and its molecular weight is between approximately 100 and approximately 1,000,000 Da.

The term “mPEG” or “mPeg” means a polydisperse or monodisperse radical of the structure

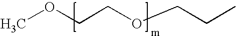

wherein m is an integer larger than 1. Thus, an mPEG wherein m is 90 has a molecular weight of 3991 Da, i.e. approximately 4 kDa. Likewise, an mPEG with an average molecular weight of 20 kDa has an average m of 454. Due to the process for producing mPEG these molecules often have a distribution of molecular weights. This distribution is described by the polydispersity index.

Due to this distribution of m, mPEG with a molecular weight of 20 kDa may also be referred to as MeO—(CH₂CH₂O)₄₀₀₋₅₀₀, mPEG with a molecular weight of 30 kDa may also be referred to as MeO—(CH₂CH₂O)₆₀₀₋₇₀₀, and mPEG with a molecular weight of 40 kDa may also be referred to as MeO—(CH₂CH₂O)₈₅₀₋₉₅₀. The heavier mPEG chains may be difficult to prepare as a single chain molecule, and they are thus made as branched mPEG. Notably, mPEG with a molecular weight of 40 kDa may be achieved with as a branched mPEG comprising to arms of 20 kDa each.

The term “polydispersity index” as used herein means the ratio between the weight average molecular weight and the number average molecular weight, as known in the art of polymer chemistry (see e.g. “Polymer Synthesis and Characterization”, J. A. Nairn, University of Utah, 2003). The polydispersity index is a number which is greater than or equal to one, and it may be estimated from Gel Permeation Chromatographic data. When the polydispersity index is 1, the product is monodisperse and is thus made up of compounds with a single molecular weight. When the polydispersity index is greater than 1 it is a measure of the polydispersity of that polymer, i.e. how broad the distribution of polymers with different molecular weights is.

The use of for example “mPEG20000” in formulas, compound names or in molecular structures indicates an mPEG residue wherein mPEG is polydisperse and has a molecular weight of approximately 20 kDa.

The polydispersity index typically increases with the molecular weight of the PEG or mPEG. When reference is made to 20 kDa PEG and in particular 20 kDa mPEG it is intended to indicate a compound (or in fact a mixture of compounds) with a polydisperisty index below 1.06, such as below 1.05, such as below 1.04, such as below 1.03, such as between 1.02 and 1.03. When reference is made to 30 kDa PEG and in particular 30 kDa mPEG it is intended to indicate a compound (or in fact a mixture of compounds) with a polydisperisty index below 1.06, such as below 1.05, such as below 1.04, such as below 1.03, such as between 1.02 and 1.03. When reference is made to 40 kDa PEG and in particular 40 kDa mPEG it is intended to indicate a compound (or in fact a mixture of compounds) with a polydisperisty index below 1.06, such as below 1.05, such as below 1.04, such as below 1.03, such as between 1.02 and 1.03

As used herein, the term “prodrug” includes biohydrolyzable amides and biohydrolyzable esters and also encompasses a) compounds in which the biohydrolyzable functionality in such a prodrug is encompassed in the compound according to the present invention, and b) compounds which may be oxidized or reduced biologically at a given functional group to yield drug substances according to the present invention. Examples of these functional groups include 1,4-dihydropyridine, N-alkylcarbonyl-1,4-dihydropyridine, 1,4-cyclohexadiene, tert-butyl, and the like.

As used herein, the term “biohydrolyzable ester” is an ester of a drug substance (in casu, a compound according to the invention) which either a) does not interfere with the biological activity of the parent substance but confers on that substance advantageous properties in vivo such as duration of action, onset of action, and the like, or b) is biologically inactive but is readily converted in vivo by the subject to the biologically active principle. The advantage is, for example increased solubility or that the biohydrolyzable ester is orally absorbed from the gut and is transformed to a compound according to the present invention in plasma. Many examples of such are known in the art and include by way of example lower alkyl esters (e.g., C₁-C₄), lower acyloxyalkyl esters, lower alkoxyacyloxyalkyl esters, alkoxyacyloxy esters, alkyl acylamino alkyl esters, and choline esters.

As used herein, the term “biohydrolyzable amide” is an amide of a drug substance (in casu, a compound according to the present invention) which either a) does not interfere with the biological activity of the parent substance but confers on that substance advantageous properties in vivo such as duration of action, onset of action, and the like, or b) is biologically inactive but is readily converted in vivo by the subject to the biologically active principle. The advantage is, for example increased solubility or that the biohydrolyzable amide is orally absorbed from the gut and is transformed to a compound according to the present invention in plasma. Many examples of such are known in the art and include by way of example lower alkyl amides, α-amino acid amides, alkoxyacyl amides, and alkylaminoalkylcarbonyl amides.

In the present context, the term “pharmaceutically acceptable salt” is intended to indicate salts which are not harmful to the patient. Such salts include pharmaceutically acceptable acid addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids and the like. Further examples of pharmaceutically acceptable inorganic or organic acid addition salts include the pharmaceutically acceptable salts listed in J. Pharm. Sci. 1977, 66, 2, which is incorporated herein by reference. Examples of metal salts include lithium, sodium, potassium, magnesium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like.

A “therapeutically effective amount” of a compound as used herein means an amount sufficient to cure, alleviate or partially arrest the clinical manifestations of a given disease and its complications. An amount adequate to accomplish this is defined as “therapeutically effective amount”. Effective amounts for each purpose will depend on e.g. the severity of the disease or injury as well as the weight, sex, age and general state of the subject. It will be understood that determining an appropriate dosage may be achieved using routine experimentation, by constructing a matrix of values and testing different points in the matrix, which is all within the ordinary skills of a trained physician or veterinary.

The term “treatment” and “treating” as used herein means the management and care of a patient for the purpose of combating a condition, such as a disease or a disorder. The term is intended to include the full spectrum of treatments for a given condition from which the patient is suffering, such as administration of the active compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of a patient for the purpose of combating the disease, condition, or disorder and includes the administration of the active compounds to prevent the onset of the symptoms or complications. The patient to be treated is preferably a mammal, in particular a human being, but it may also include animals, such as dogs, cats, cows, sheep and pigs.

### DESCRIPTION OF THE INVENTION

In one embodiment, the present invention relates to GH to which a group has been conjugated at the N-terminal, i.e. to compounds according to formula (I)

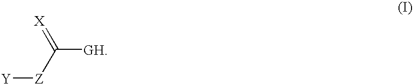

Said compounds have improved pharmacological properties compared to the corresponding un-conjugated GH, also referred to as the parent GH. Examples of such pharmacological properties include functional in vivo half-life, immunogenicity, renal filtration, protease protection and albumin binding.

The term “functional in vivo half-life” is used in its normal meaning, i.e., the time at which 50% of the biological activity of the GH or conjugated GH are still present in the body/target organ, or the time at which the activity of the GH or GH conjugate is 50% of its initial value. As an alternative to determining functional in vivo half-life, “in vivo plasma half-life” may be determined, i.e., the time at which 50% of the GH or GH conjugate circulate in the plasma or bloodstream prior to being cleared. Determination of plasma half-life is often more simple than determining functional half-life and the magnitude of plasma half-life is usually a good indication of the magnitude of functional in vivo half-life. Alternative terms to plasma half-life include serum half-life, circulating half-life, circulatory half-life, serum clearance, plasma clearance, and clearance half-life.

The term “increased” as used in connection with the functional in vivo half-life or plasma half-life is used to indicate that the relevant half-life of the GH conjugate is statistically significantly increased relative to that of the parent GH, as determined under comparable conditions. For instance the relevant half-life may be increased by at least about 25%, such as by at lest about 50%, e.g., by at least about 100%, 150%, 200%, 250%, or 500%. In one embodiment, the compounds of the present invention exhibit an increase in half-life of at least about 5 h, preferably at least about 24 h, more preferably at least about 72 h, and most preferably at least about 7 days, relative to the half-life of the parent GH.

Measurement of in vivo plasma half-life can be carried out in a number of ways as described in the literature. An increase in in vivo plasma half-life may be quantified as a decrease in clearance (CL) or as an increase in mean residence time (MRT). Conjugated GH of the present invention for which the CL is decreased to less than 70%, such as less than 50%, such than less than 20%, such than less than 10% of the CL of the parent GH as determined in a suitable assay is said to have an increased in vivo plasma half-life. Conjugated GH of the present invention for which MRT is increased to more than 130%, such as more than 150%, such as more than 200%, such as more than 500% of the MRT of the parent GH in a suitable assay is said to have an increased in vivo plasma half-life. Clearance and mean residence time can be assessed in standard pharmacokinetic studies using suitable test animals. It is within the capabilities of a person skilled in the art to choose a suitable test animal for a given protein. Tests in human, of course, represent the ultimate test. Suitable text animals include normal, Sprague-Dawley male rats, mice and cynomolgus monkeys. Typically the mice and rats are in injected in a single subcutaneous bolus, while monkeys may be injected in a single subcutaneous bolus or in a single iv dose. The amount injected depends on the test animal. Subsequently, blood samples are taken over a period of one to five days as appropriate for the assessment of CL and MRT. The blood samples are conveniently analysed by ELISA techniques.

The term “Immunogenicity” of a compound refers to the ability of the compound, when administered to a human, to elicit a deleterious immune response, whether humoral, cellular, or both. In any human sub-population, there may exist individuals who exhibit sensitivity to particular administered proteins. Immunogenicity may be measured by quantifying the presence of growth hormone antibodies and/or growth hormone responsive T-cells in a sensitive individual, using conventional methods known in the art. In one embodiment, the conjugated GH of the present invention exhibit a decrease in immunogenicity in a sensitive individual of at least about 10%, preferably at least about 25%, more preferably at least about 40% and most preferably at least about 50%, relative to the immunogenicity for that individual of the parent GH.

The term “protease protection” or “protease protected” as used herein is intended to indicate that the conjugated GH of the present invention is more resistant to the plasma peptidase or proteases than is the parent GH. Protease and peptidase enzymes present in plasma are known to be involved in the degradation of circulating proteins, such as e.g. circulating peptide hormones, such as growth hormone.

Resistance of a protein to degradation by for instance dipeptidyl aminopeptidase IV (DPPIV) is determined by the following degradation assay: Aliquots of the protein (5 nmol) are incubated at 37° C. with 1 μL of purified dipeptidyl aminopeptidase IV corresponding to an enzymatic activity of 5 mU for 10-180 minutes in 100 μL of 0.1 M triethylamine-HCl buffer, pH 7.4. Enzymatic reactions are terminated by the addition of 5 μL of 10% trifluoroacetic acid, and the protein degradation products are separated and quantified using HPLC analysis. One method for performing this analysis is: The mixtures are applied onto a Vydac C18 widepore (30 nm pores, 5 μm particles) 250×4.6 mm column and eluted at a flow rate of 1 ml/min with linear stepwise gradients of acetonitrile in 0.1% trifluoroacetic acid (0% acetonitrile for 3 min, 0-24% acetonitrile for 17 min, 24-48% acetonitrile for 1 min) according to Siegel et al., Regul. Pept. 1999; 79:93-102 and Mentlein et al. Eur. J. Biochem. 1993; 214:829-35. Proteins and their degradation products may be monitored by their absorbance at 220 nm (peptide bonds) or 280 nm (aromatic amino acids), and are quantified by integration of their peak areas related to those of standards. The rate of hydrolysis of a protein by dipeptidyl aminopeptidase IV is estimated at incubation times which result in less than 10% of the peptide being hydrolysed. In one embodiment, the rate of hydrolysis of the GH conjugate is less than 70%, such as less than 40%, such as less than 10% of that of the parent GH.

The most abundant protein component in circulating blood of mammalian species is serum albumin, which is normally present at a concentration of approximately 3 to 4.5 grams per 100 milliters of whole blood. Serum albumin is a blood protein of approximately 70,000 daltons which has several important functions in the circulatory system. It functions as a transporter of a variety of organic molecules found in the blood, as the main transporter of various metabolites such as fatty acids and bilirubin through the blood, and, owing to its abundance, as an osmotic regulator of the circulating blood. Serum albumin has a half-life of more than one week, and one approach to increasing the plasma half-life of proteins has been to conjugate to the protein a group that binds to serum albumin. Albumin binding property may be determined as described in J. Med. Chem., 43, 2000, 1986-1992, which is incorporated herein by reference.

In one embodiment, GH is human growth hormone (hGH) which has an amino acid sequence as set forth in SEQ ID NO 1.

In one embodiment, GH is a variant of hGH, wherein a variant is understood to be the compound obtained by substituting one or more amino acid residues in the hGH sequence with another natural or unnatural amino acid; and/or by adding one or more natural or unnatural amino acids to the hGH sequence; and/or by deleting one or more amino acid residue from the hGH sequence, wherein any of these steps may optionally be followed by further derivatization of one or more amino acid residue. In particular, such substitutions are conservative in the sense that one amino acid residue is substituted by another amino acid residue from the same group, i.e. by another amino acid residue with similar properties. Amino acids may conveniently be divided in the following groups based on their properties: Basic amino acids (such as arginine, lysine, histidine), acidic amino acids (such as glutamic acid and aspartic acid), polar amino acids (such as glutamine, cysteine and asparagine), hydrophobic amino acids (such as leucine, isoleucine, proline, methionine and valine), aromatic amino acids (such as phenylalanine, tryptophan, tyrosine) and small amino acids (such as glycine, alanine, serine and threonine.).

In one embodiment, GH has at least 80%, such as at least 85%, such as at least 90%, such as at least 95% identity with hGH. In one embodiment, said identities to hGH is coupled to at least 20%, such as at least 40%, such as at least 60%, such as at least 80% of the growth hormone activity of hGH as determined in assay I herein.

The term “identity” as known in the art, refers to a relationship between the sequences of two or more proteins, as determined by comparing the sequences. In the art, “identity” also means the degree of sequence relatedness between proteins, as determined by the number of matches between strings of two or more amino acid residues. “Identity” measures the percent of identical matches between the smaller of two or more sequences with gap alignments (if any) addressed by a particular mathematical model or computer program (i.e., “algorithms”). Identity of related proteins can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math., 48:1073 (1988).

Preferred methods to determine identity are designed to give the largest match between the sequences tested. Methods to determine identity are described in publicly available computer programs. Preferred computer program methods to determine identity between two sequences include the GCG program package, including GAP (Devereux et al., Nucl. Acid. Res., 12:387 (1984); Genetics Computer Group, University of Wisconsin, Madison, Wis.), BLASTP, BLASTN, and FASTA (Altschul et al., J. Mol. Biol., 215:403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well known Smith Waterman algorithm may also be used to determine identity.

For example, using the computer algorithm GAP (Genetics Computer Group, University of Wisconsin, Madison, Wis.), two proteins for which the percent sequence identity is to be determined are aligned for optimal matching of their respective amino acids (the “matched span”, as determined by the algorithm). A gap opening penalty (which is calculated as 3.times. the average diagonal; the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. A standard comparison matrix (see Dayhoff et al., Atlas of Protein Sequence and Structure, vol. 5, supp. 3 (1978) for the PAM 250 comparison matrix; Henikoff et al., Proc. Natl. Acad. Sci USA, 89:10915-10919 (1992) for the BLOSUM 62 comparison matrix) is also used by the algorithm.

Preferred parameters for a protein sequence comparison include the following:

Algorithm: Needleman et al., J. Mol. Biol, 48:443-453 (1970); Comparison matrix: BLOSUM 62 from Henikoff et al., Proc. Natl. Acad. Sci. USA, 89:10915-10919 (1992); Gap Penalty: 12, Gap Length Penalty: 4, Threshold of Similarity: 0. The GAP program is useful with the above parameters. The aforementioned parameters are the default parameters for protein comparisons (along with no penalty for end gaps) using the GAP algorithm.

In one embodiment, GH is hGH extended with up to 100 amino acid residues at the N-terminal. In particular, said extension is up to 50, such as up to 40, such as up to 20, such as up to 10, such as up to 5, such as 1, 2 or 3 amino acid residues.

In one embodiment, embodiment i), X represents two hydrogens in which case compounds of formula (I) take the form of formula (I′)

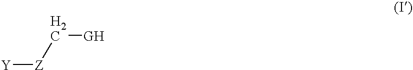

In a further embodiment of embodiment i), Y represents

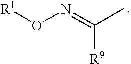

In a further embodiment of embodiment i), R⁹ represents hydrogen.

In a further embodiment to embodiment i), R¹ represents MeO—(CH₂CH₂O)₁₀₀₋₁₀₀₀-E- or [MeO—(CH₂CH₂O)₁₀₀₋₁₀₀₀]—C(═O)NH—(CH₂)₄CH[MeO—(CH₂CH₂O)₁₀₀₋₁₀₀₀C(═O)NH]E-.

In an further embodiment of embodiment i), R¹ represents MeO—(CH₂CH₂O)₄₀₀₋₁₀₀₀-E- or [MeO—(CH₂CH₂O)₃₀₀₋₆₀₀]—C(═O)NH—(CH₂)₄CH[MeO—(CH₂CH₂O)₃₀₀₋₆₀₀—C(═O)NH]E-. In particular, R¹ may represent MeO—(CH₂CH₂O)₄₀₀₋₅₀₀-E-, MeO—(CH₂CH₂O)₆₀₀₋₇₀₀-E- or MeO—(CH₂CH₂O)₈₅₀₋₉₅₀-E-; or [MeO—(CH₂CH₂O)₄₀₀₋₅₀₀]—C(═O)NH—(CH₂)₄CH[MeO—(CH₂CH₂O)₄₀₀₅₀₀—C(═O)NH]E-.

In a further embodiment to embodiment i), E represents —C(═O)NH(CH₂)₂₋₃₀— or —(CH₂)₁₋₃₀C(═O)NH(CH₂)₂₋₃₀—.

In a further embodiment to embodiment i), E represents —C(═O)NH(CH₂)₂₋₁₀ or —(CH₂)₁₋₁₀C(═O)NH(CH₂)₂₋₁₀—. In particular, E may represent —C(═O)NH(CH₂)₄ or —(CH₂)₃C(═O)NH(CH₂)₄—.

In a further embodiment to embodiment i), R¹ is selected from

### MeO—(CH₂CH₂O)₄₀₀₋₅₀₀—CH₂CH₂CH₂—C(═O)—NH—(CH₂)₄—,

### MeO—(CH₂CH₂O)₆₀₀₋₇₀₀—CH₂CH₂CH₂—C(═O)—NH—(CH₂)₄—,

### MeO—(CH₂CH₂O)₈₅₀₋₉₅₀—CH₂CH₂CH₂—C(═O)—NH—(CH₂)₄—, and

### MeO—(CH₂CH₂O)₄₀₀₋₅₀₀—C(═O)NH—(CH₂)₄CH[MeO—(CH₂CH₂O)₄₀₀₋₅₀₀—C(═O)NH]—.

In a further embodiment of embodiment i), Z represents —CH₂—O—(CH₂)₁₋₅ or —CH—O—(C₆H₄)—. In particular, Z may represent —CH₂—O—(CH₂)₃, —CH₂—O—(CH₂)₄, —CH₂—O—(CH₂)₅, —CH₂—O-(1,4-C₆H₄)— or —CH₂—O-(1,3-C₆H₄)—.

In one embodiment, embodiment ii), X represents O and Z represents a bond, in which case compounds of formula (I) take the form of formula (I″)

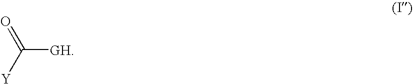

In one embodiment of embodiment ii), Y represents

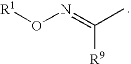

In one embodiment of embodiment ii), R⁹ represents hydrogen.

In one embodiment of embodiment ii), R¹ represents MeO—(CH₂CH₂O)₁₀₀₋₁₀₀₀-E-, [MeO—(CH₂CH₂O)₁₀₀₋₁₀₀₀]—C(═O)NH—(CH₂)₄CH[MeO—(CH₂CH₂O)₁₀₀₋₁₀₀₀—C(═O)NH]E-, Me-(CH₂)₁₀₋₃₀-E-, (5-tetrazolyl)-(CH₂)₁₀₋₃₀-E-, (5-tetrazolyl)₂CH—(CH₂)₁₀₋₃₀-E-, (5-tetrazolyl)-(CH₂)₁₋₃₀—C(═O)—NH—S(═O)₂—(CH₂)₂₋₁₀-E-, (5-tetrazoly)-(C₆H₄)—O—(CH₂)₁₋₃₀, (5-tetrazoly)-(C₆H₄)—(C₆H₄)—O—(CH₂)₁₋₃₀, GH-C(═O)—CH═N—(OCH₂CH₂)₁₋₁₀—, or cibacronyl-E-.

In one embodiment of embodiment ii), R¹ represents MeO—(CH₂CH₂O)₄₀₀₋₁₀₀₀-E-, [MeO—(CH₂CH₂O)₄₀₀₋₈₀₀]—C(═O)NH—(CH₂)₄CH[MeO—(CH₂CH₂O)₄₀₀₋₈₀₀—C(═O)NH]E-, Me-(CH₂)₁₀₋₂₀-E-, (5-tetrazolyl)-(CH₂)₁₋₂₀-E-, (5-tetrazolyl)₂CH—(CH₂)₁₋₂₀-E-, (5-tetrazolyl)-(CH₂)₁₋₂₀—C(═O)—NH—S(═O)₂—(CH₂)₂₋₅-E-, (5-tetrazoly)-(C₆H₄)—O—(CH₂)₁₋₂₀, (5-tetrazoly)-(C₆H₄)—(C₆H₄)—O—(CH₂)₁₋₂₀—, GH-C(═O)—CH═N—(OCH₂CH₂)₁₋₈—, or cibacronyl-E-.

In one embodiment, R¹ represents MeO—(CH₂CH₂O)₄₀₀₋₅₀₀-E-, MeO—(CH₂CH₂O)₆₀₀₋₇₀₀-E-, MeO—(CH₂CH₂O)₈₅₀₋₉₅₀-E-, [MeO—(CH₂CH₂O)₄₀₀₅₀₀]—C(═O)NH—(CH₂)₄CH[MeO—(CH₂CH₂O)₄₀₀₋₅₀₀—C(═O)NH]E-, [MeO—(CH₂CH₂O)₆₀₀₋₇₀₀]—C(═O)NH—(CH₂)₄CH[MeO—(CH₂CH₂O)₆₀₀₇₀₀—C(═O)NH]E-, Me-(CH₂)₁₂-E-, Me-(CH₂)₁₀₂₀-E-, Me-(CH₂)₁₄-E-, Me-(CH₂)₁₆-E-, Me-(CH₂)₁₈-E-, (5-tetrazolyl)-(CH₂)₁₅-E-, (5-tetrazolyl)₂CH—(CH₂)₁₄-E-, (5-tetrazolyl)-(CH₂)₁₅—C(═O)—NH—S(═O)₂—(CH₂)₄-E-, (5-tetrazoly)-(C₆H₄)—O—(CH₂)₁₅, (5-tetrazoly)-(C₆H₄)—(C₆H₄)—O—(CH₂)₁₅—, or cibacronyl-E-.

In one embodiment, R¹ represents

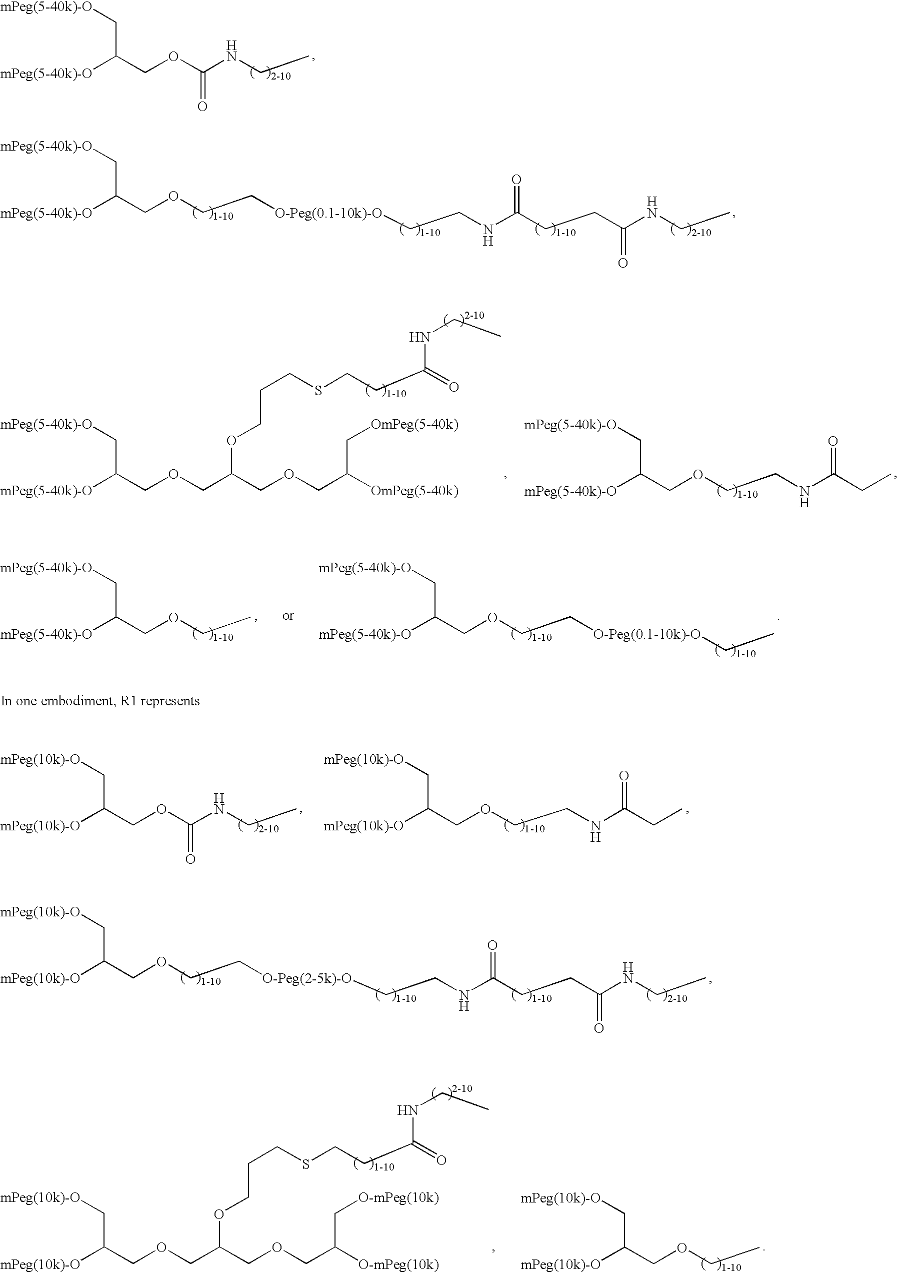

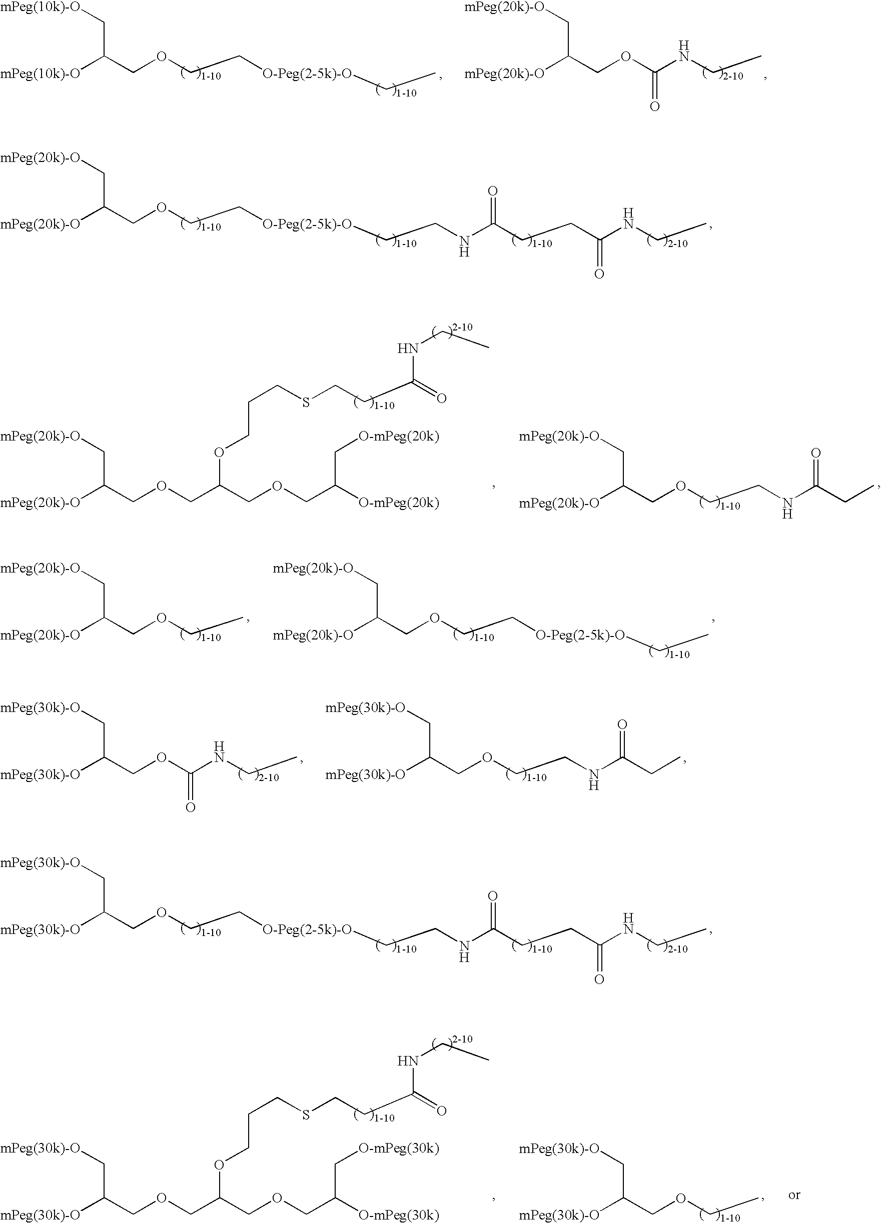

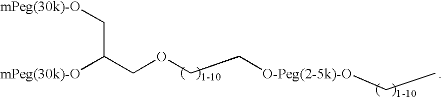

in one embodiment of embodiment (ii), Y represents

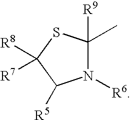

In one embodiment, R⁶ and R⁹ represent hydrogen, R⁷ and R⁸ independently represent hydrogen or methyl, and R⁵ represents

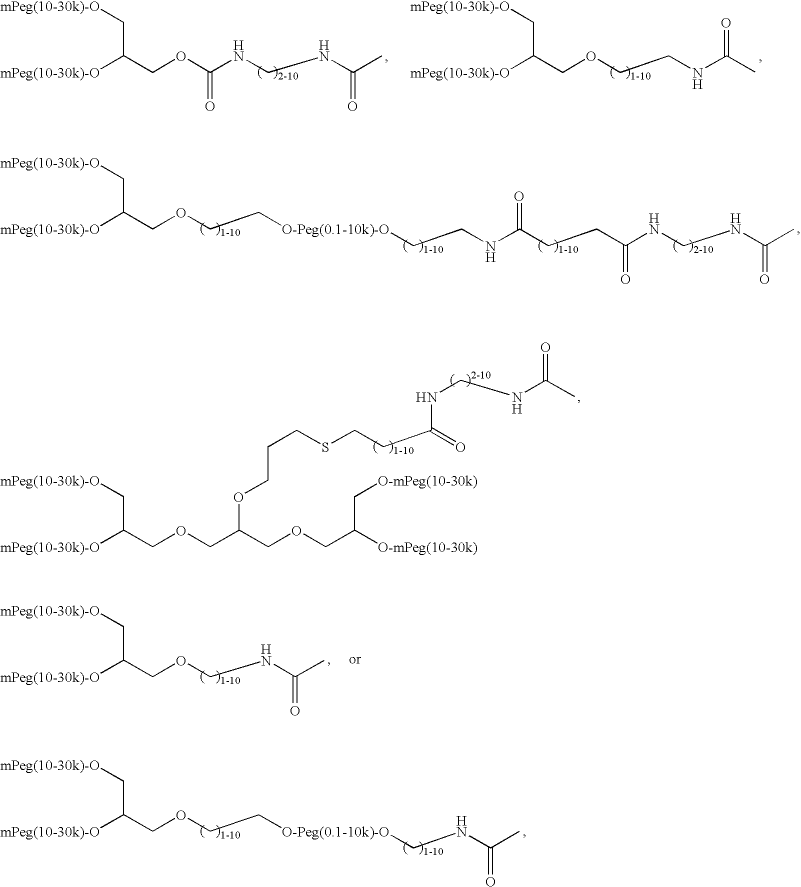

wherein mPeg(10-30k)=mPeg(10k), mPeg(20k), and mPeg(30k) are separate and specific embodiments for each mPeg(10-30k) and any specific combination thereof.

In one embodiment, R⁶ and R⁹ represent hydrogen, R⁷ and R⁸ independently represent hydrogen or methyl, and R⁵ represents

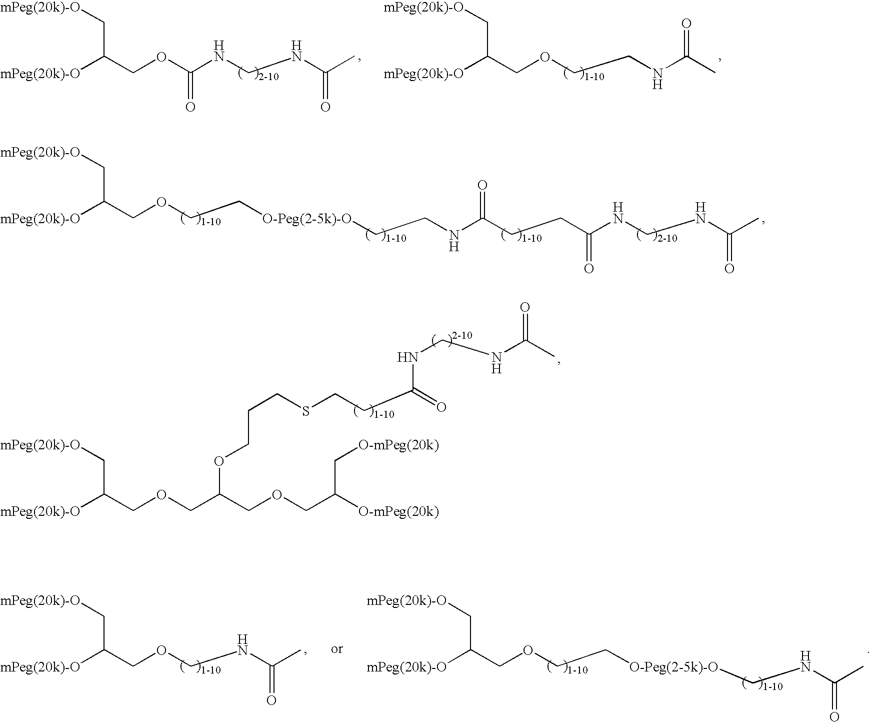

In a further embodiment of embodiment ii), E represents —C(═O)NH(CH₂)₂₋₃₀—,

—(CH₂)₁₋₃₀C(═O)NH(CH₂)₂₋₃₀—,

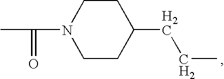

—C(═O)NH—[(CH₂CH₂O)₁₋₁₀—(CH₂)₁₋₅—C(═O)]₁₋₅NH(CH₂)₂₋₃₀—, or a bond.

In a further embodiment of embodiment ii), E represents —C(═O)NH(CH₂)₂₋₁₀—,

—(CH₂)₁₋₁₀C(═O)NH(CH₂)₂₋₁₀—,

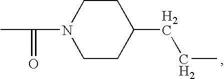

—C(═O)NH—[(CH₂CH₂O)₁₋₅—(CH₂)₁—C(═O)]₁₋₄NH(CH₂)₂₋₁₀—, or a bond. Particular examples of E includes —C(═O)NH(CH₂)₄—, —(CH₂)₃C(═O)NH(CH₂)₄,

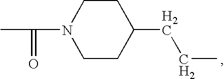

—C(═O)NH—[(CH₂CH₂O)₂—(CH₂)₁—C(═O)]₁ NH(CH₂)₄—, —C(═O)NH-[(CH₂CH₂O)₂—(CH₂)₁—C(═O)]₂NH(CH₂)₄—, —C(═O)NH—[(CH₂CH₂O)₂—(CH₂)₁—C(═O)]₃NH(CH₂)₄—, and a bond.

In a further embodiment to embodiment ii), R¹ represents

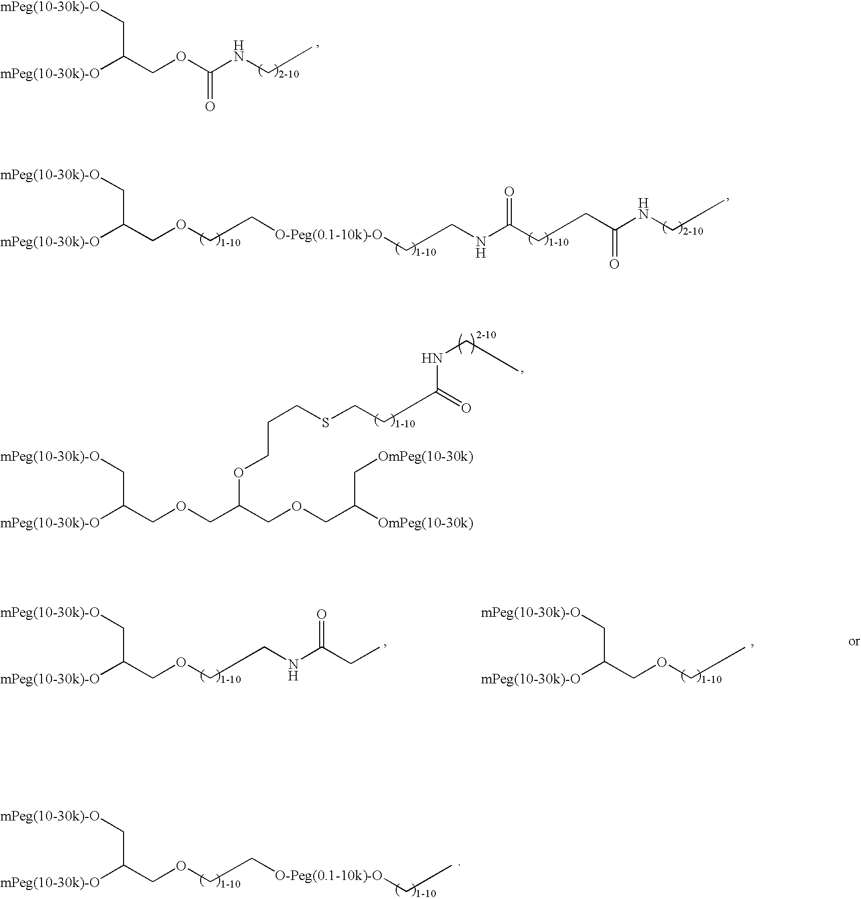

In a further embodiment to embodiment ii), R¹ represents

### MeO—(CH₂CH₂O)₄₀₀₋₅₀₀—CH₂CH₂CH₂—C(═O)—NH—(CH₂)₄—,

### MeO—(CH₂CH₂O)₆₀₀₋₇₀₀—CH₂CH₂CH₂—C(═O)—NH—(CH₂)₄—,

### MeO—(CH₂CH₂O)₈₅₀₋₉₅₀—CH₂CH₂CH₂—C(═O)—NH—(CH₂)₄—,

MeO—(CH₂CH₂O)₄₀₀₋₅₀₀—C(═O)NH—(CH₂)₄CH[MeO—(CH₂CH₂O)₄₀₀₋₅₀₀—C(═O)NH]—C(═O)NH—(CH₂)₄—,
MeO—(CH₂CH₂O)₆₀₀₋₇₀₀—C(═O)NH—(CH₂)₄CH[MeO—(CH₂CH₂O)₆₀₀₋₇₀₀—C(═O)NH]—C(═O)NH—(CH₂)₄—,

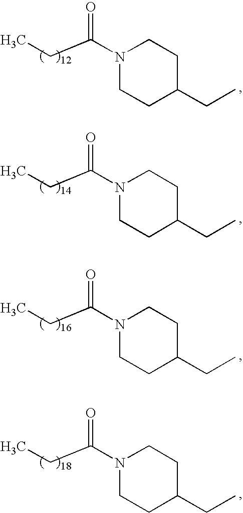

(5-tetrazolyl)-(CH₂)₁₅—C(═O)—NH—(CH₂)₄—,
(5-tetrazolyl)-(CH₂)₁₅—C(═O)—NH—S(═O)₂—(CH₂)₃—C(═O)—NH—(CH₂)₄—,
(5-tetrazolyl)-(CH₂)₁₅—C(═O)—NH—(CH₂CH₂—O)₂—CH₂—C(═O)NH—(CH₂)₄—,
(5-tetrazolyl)-(CH₂)₁₅—C(═O)-[NH—(CH₂CH₂—O)₂—CH₂—C(═O)]₂NH—(CH₂)₄—,
(5-tetrazolyl)-(CH₂)₁₅—C(═O)-[NH—(CH₂CH₂—O)₂—CH₂—C(═O)]₃NH—(CH₂)₄—,
(5-tetrazolyl)₂CH—(CH₂)₁₄—C(═O)—NH—(CH₂)₄—
(5-tetrazolyl)(1,4-C₆H₄)—O—(CH₂)₁₅—C(═O)—NH—(CH₂)₄—
(5-tetrazolyl)(1,4-C₆H₄)₂—O—(CH₂)₁₅—C(═O)—NH—(CH₂)₄—

### GH-C(═O)—CH₂—CH═N—(O—CH₂CH₂)₂—,

### GH-C(═O)—CH₂—CH═N—(O—CH₂CH₂)₃—,

### GH-C(═O)—CH₂—CH═N—(O—CH₂CH₂)₄—,

### GH-C(═O)—CH₂—CH═N—(O—CH₂CH₂)₅—,

### GH-C(═O)—CH₂—CH═N—(O—CH₂CH₂)₆—,

cibacronyl-NH—(CH₂)₄—,

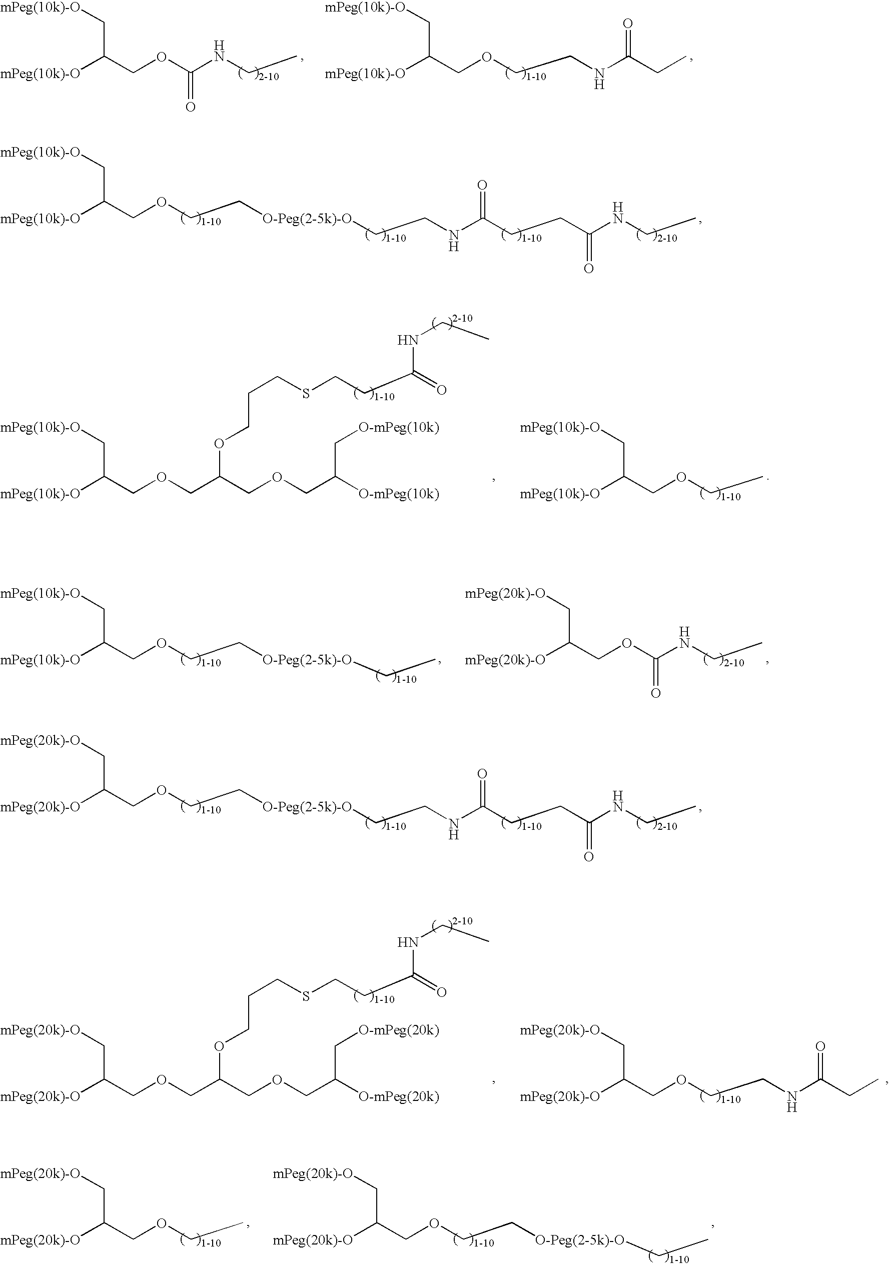

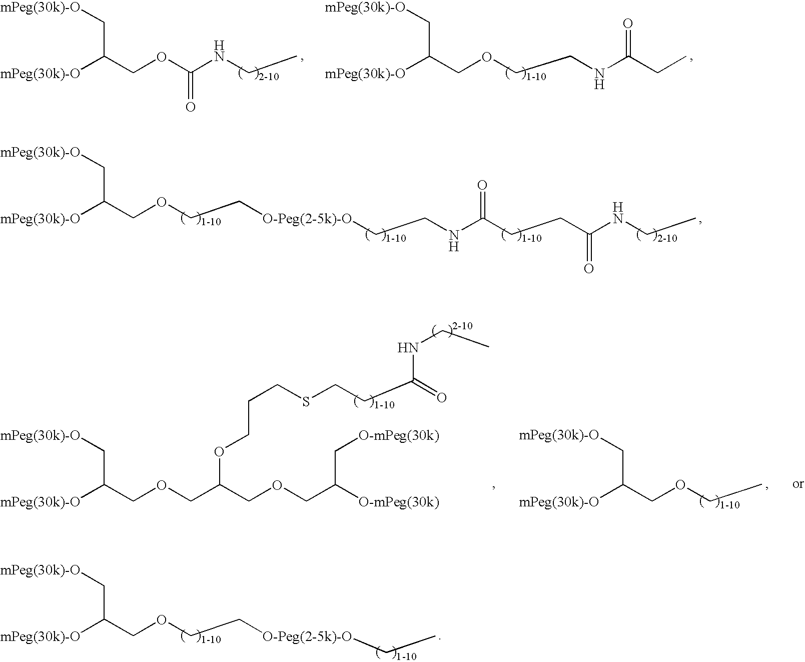

Particular examples of compounds according to formula I include

[MeO—(CH₂CH₂O)₄₀₀₋₅₀₀—CH₂CH₂—NH—C(═O)—O—CH₂]₂CH—O—(CH₂)₃—C(═O)—NH—(CH₂)₄—O—N═CH—C(═O)-GH,
[MeO—(CH₂CH₂O)₆₀₀₋₇₅₀](CH₂)₃—C(═O)NH—(CH₂)₄CH[MeO—(CH₂CH₂O)₆₀₀₋₇₅₀—(CH₂)₃C(═O)NH]—C(═O)NH—(CH₂)₄—O—N═CH—C(═O)-GH,

### MeO—(CH₂CH₂O)₄₀₀₋₅₀₀—CH₂CH₂CH₂—C(═O)—NH—(CH₂)₄—O—N═CH—C(═O)-GH,

### MeO—(CH₂CH₂O)₆₀₀₋₇₀₀—CH₂CH₂CH₂—C(═O)—NH—(CH₂)₄—O—N═CH—C(═O)-GH,

### MeO—(CH₂CH₂O)₈₅₀₋₉₅₀—CH₂CH₂CH₂—C(═O)—NH—(CH₂)₄—O—N═CH—C(═O)-GH,

[MeO—(CH₂CH₂O)₄₀₀₋₅₀₀]—C(═O)NH—(CH₂)₄CH[MeO—(CH₂CH₂O)₄₀₀₋₅₀₀—C(═O)NH]—C(═O)NH—(CH₂)₄—O—N═CH—C(═O)-GH,
[MeO—(CH₂CH₂O)₆₀₀₋₇₀₀]—C(═O)NH—(CH₂)₄CH[MeO—(CH₂CH₂O)₆₀₀₋₇₀₀—C(═O)NH]—C(═O)NH—(CH₂)₄—O—N═CH—C(═O)-GH,

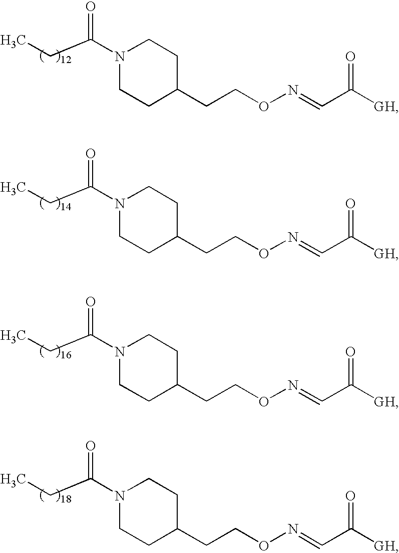

(5-tetrazolyl)-(CH₂)₁₅—C(═O)—NH—(CH₂)₄—O—N═CH—C(═O)-GH,
(5-tetrazolyl)-(CH₂)₁₅—C(═O)—NH—S(═O)₂—(CH₂)₃—C(═O)—NH—(CH₂)₄—O—N═CH—C(═O)-GH,
(5-tetrazolyl)-(CH₂)₁₅—C(═O)—NH—(CH₂CH₂—O)₂—CH₂—C(═O)NH—(CH₂)₄—O—N═CH—C(═O)-GH,
(5-tetrazolyl)-(CH₂)₁₋₅—C(═O)—[NH—(CH₂CH₂—O)₂—CH₂—C(═O)]₂NH—(CH₂)₄—O—N═CH—C(═O)-GH.
(5-tetrazolyl)-(CH₂)₁₅—C(═O)-[NH—(CH₂CH₂—O)₂—CH₂—C(═O)]₃NH—(CH₂)₄—O—N═CH—C(═O)-GH,
(5-tetrazolyl)₂CH—(CH₂)₁₄—C(═O)—NH—(CH₂)₄—O—N═CH—C(═O)-GH,
(5-tetrazolyl)(1,4-C₆H₄)—O—(CH₂)₁₅—C(═O)—NH—(CH₂)₄—O—N═CH—C(═O)-GH,
(5-tetrazolyl)(1,4-C₆H₄)₂—O—(CH₂)₁₅—C(═O)—NH—(CH₂)₄—O—N═CH—C(═O)-GH,

### GH-C(═O)—CH═N—(O—CH₂CH₂)₂—O—N═CH—C(═O)-GH,

### GH-C(═O)—CH═N—(O—CH₂CH₂)₃—O—N═CH—C(═O)-GH,

### GH-C(═O)—CH═N—(O—CH₂CH₂)₄—O—N═CH—C(═O)-GH,

### GH-C(═O)—CH═N—(O—CH₂CH₂)₅—O—N═CH—C(═O)-GH,

### GH-C(═O)—CH═N—(O—CH₂CH₂)₆—O—N═CH—C(═O)-GH,

cibacronyl-NH—(CH₂)₄—O—N═CH—C(═O)-GH,
MeO—(CH₂CH₂O)₄₀₀₋₅₀₀—CH₂CH₂CH₂—C(═O)—NH—(CH₂)₄—O—N═CH—CH₂—O—(CH₂)₄-GH,
MeO—(CH₂CH₂O)₆₀₀₋₇₀₀—CH₂CH₂CH₂—C(═O)—NH—(CH₂)₄—O—N═CH—CH₂—O—(CH₂)₄-GH,
MeO—(CH₂CH₂O)₈₅₀₋₉₅₀—CH₂CH₂CH₂—C(═O)—NH—(CH₂)₄—O—N═CH—CH₂—O—(CH₂)₄-GH,
[MeO—(CH₂CH₂O)₄₀₀₋₅₀₀]—C(═O)NH—(CH₂)₄CH[MeO—(CH₂CH₂O)₄₀₀₋₅₀₀—C(═O)NH]—C(═O)NH—(CH₂)₄—O—N═CH—CH₂—O—(CH₂)₄-GH,
MeO—(CH₂CH₂O)₄₀₀₋₅₀₀—CH₂CH₂CH₂—C(═O)—NH—(CH₂)₄—O—N═CH—CH₂—O—(CH₂)₅-GH,
MeO—(CH₂CH₂O)₆₀₀₋₇₀₀—CH₂CH₂CH₂—C(═O)—NH—(CH₂)₄—O—N═CH—CH₂—O—(CH₂)₅-GH,
MeO—(CH₂CH₂O)₈₅₀₋₉₅₀—CH₂CH₂CH₂—C(═O)—NH—(CH₂)₄—O—N═CH—CH₂—O—(CH₂)₅-GH,
[MeO—(CH₂CH₂O)₄₀₀₋₅₀₀]—C(═O)NH—(CH₂)₄CH[MeO—(CH₂CH₂O)₄₀₀₋₅₀₀—C(═O)NH]—C(═O)NH—(CH₂)₄—O—N═CH—CH₂—O—(CH₂)₅-GH,
MeO—(CH₂CH₂O)₄₀₀₋₅₀₀—CH₂CH₂CH₂—C(═O)—NH—(CH₂)₄—O—N═CH—CH₂—O—(CH₂)₆-GH,
MeO—(CH₂CH₂O)₆₀₀₋₇₀₀—CH₂CH₂CH₂—C(═O)—NH—(CH₂)₄—O—N═CH—CH₂—O—(CH₂)₆-GH,
MeO—(CH₂CH₂O)₈₅₀₋₉₅₀—CH₂CH₂CH₂—C(═O)—NH—(CH₂)₄—O—N═CH—CH₂—O—(CH₂)₆-GH,
[MeO—(CH₂CH₂O)₄₀₀₋₅₀₀]—C(═O)NH—(CH₂)₄CH[MeO—(CH₂CH₂O)₄₀₀₋₅₀₀—C(═O)NH]—C(═O)NH—(CH₂)₄—O—N═CH—CH₂—O—(CH₂)₆-GH,
MeO—(CH₂CH₂O)₄₀₀₋₅₀₀—CH₂CH₂CH₂—C(═O)—NH—(CH₂)₄—O—N═CH—CH₂—O-(1,4-C₆H₄)CH₂-GH,
MeO—(CH₂CH₂O)₆₀₀₋₇₀₀—CH₂CH₂CH₂—C(═O)—NH—(CH₂)₄—O—N═CH—CH₂—O-(1,4-C₆H₄)CH₂-GH,
MeO—(CH₂CH₂O)₈₅₀₋₉₅₀—CH₂CH₂CH₂—C(═O)—NH—(CH₂)₄—O—N═CH—CH₂—O-(1,4-C₆H₄)CH₂-GH,
[MeO—(CH₂CH₂O)₄₀₀₋₅₀₀]—C(═O)NH—(CH₂)₄CH[MeO—(CH₂CH₂O)₄₀₀₋₅₀₀—C(═O)NH]—C(═O)NH—(CH₂)₄—O—N═CH—CH₂—O-(1,4-C₆H₄)CH₂-GH,
MeO—(CH₂CH₂O)₄₀₀₋₅₀₀—CH₂CH₂CH₂—C(═O)—NH—(CH₂)₄—O—N═CH—CH₂—O-(1,3-C₆H₄)CH₂-GH,
MeO—(CH₂CH₂O)₆₀₀₋₇₀₀—CH₂CH₂CH₂—C(═O)—NH—(CH₂)₄—O—N═CH—CH₂—O-(1,3-C₆H₄)CH₂-GH,
MeO—(CH₂CH₂O)₈₅₀₋₉₅₀—CH₂CH₂CH₂—C(═O)—NH—(CH₂)₄—O—N═CH—CH₂—O-(1,3-C₆H₄)CH₂-GH,
[MeO—(CH₂CH₂O)₄₀₀₋₅₀₀]—C(═O)NH—(CH₂)₄CH[MeO—(CH₂CH₂O)₄₀₀₋₅₀₀—C(═O)NH]—C(═O)NH—(CH₂)₄—O—N═CH—CH₂—O-(1,3-C₆H₄)CH₂-GH,

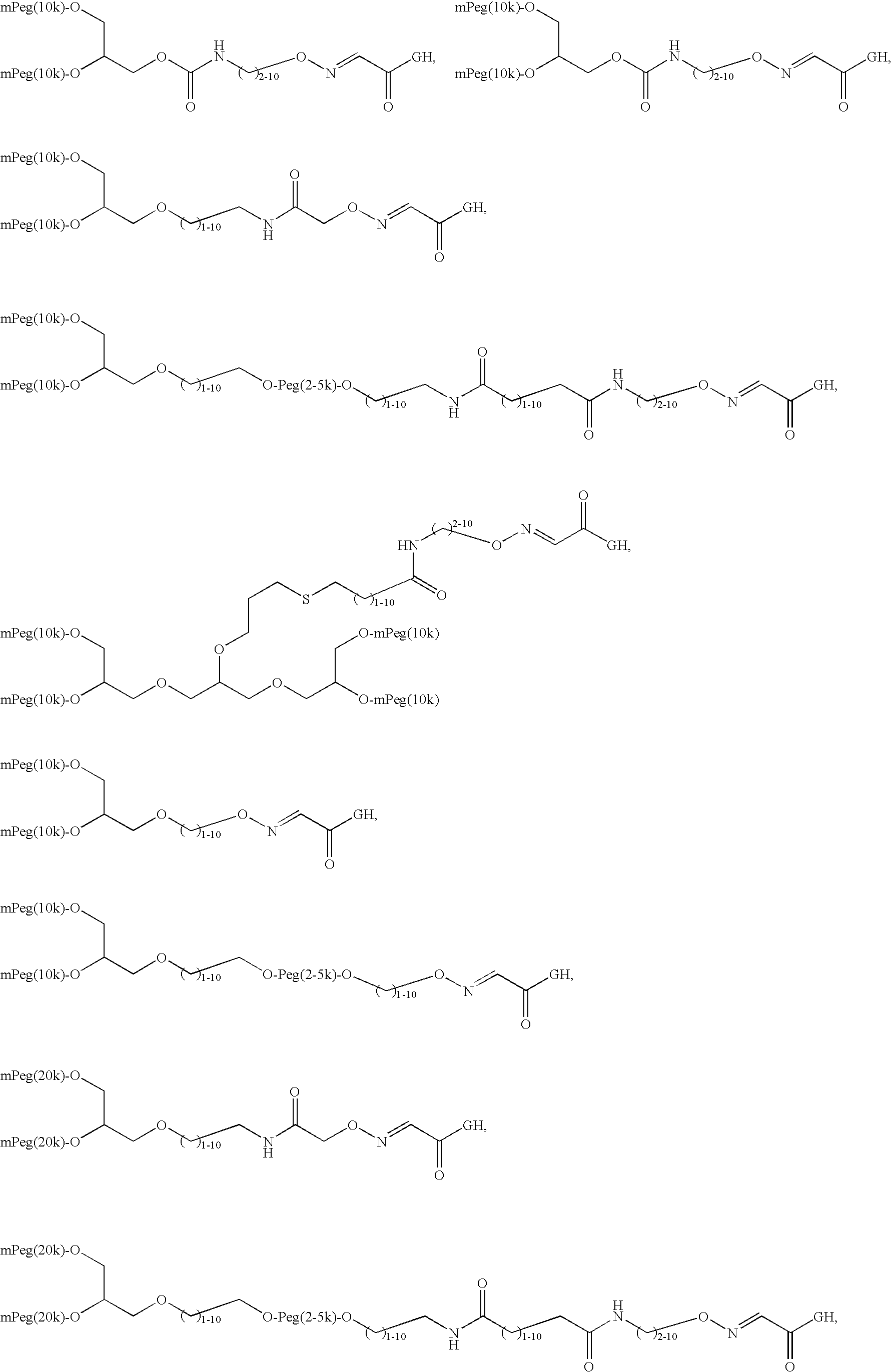

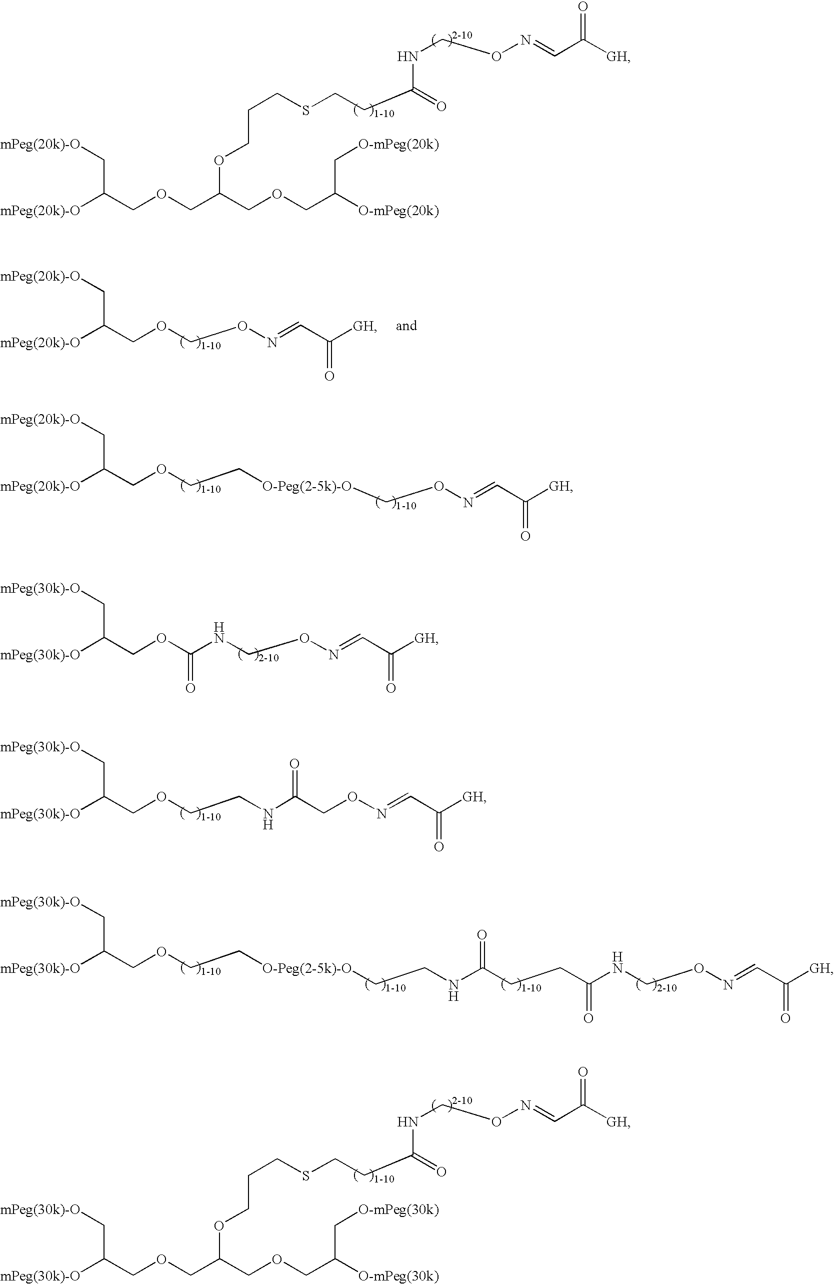

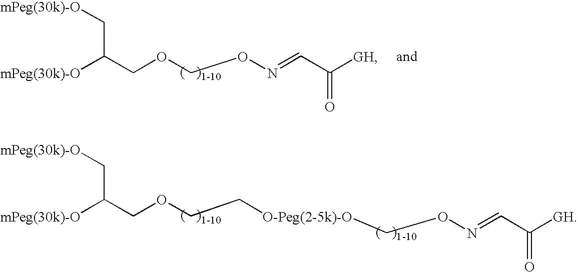

In one embodiment, the GH in the above compounds is hGH.

In an additional or alternative embodiment, the invention provides the methods and compounds described herein, wherein each instance of “mPEG” is replaced by an alkoxy-PEG or “aPEG” compound of the formula

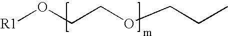

wherein m is an integer larger than 1. Each R¹ can be any suitable C₁₋₁₀ alkyl group, branched or (for C₃₋₁₀) unbranched, including, but not limited to, methyl, ethyl, and propyl, and butyl.

In one embodiment, the invention relates a method for preparing compounds of formula (I) or (I′), the method comprising the steps of

(a) reductive alkylation of the N-terminal amino group in GH with a compound of the formula Q-Z-CH═O, wherein Q is a functional group which can be converted into an aldehyde or a ketone by treatment with a suitable reagent, to obtain a compound of formula II.

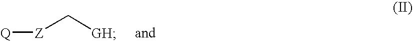

(b) conversion of the functional group Q in the compound of formula (II) into the aldehyde or ketone of formula (III′)

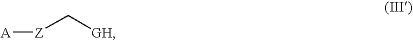

wherein A represents an aldehyde or ketone moiety; and
(c) condensation of the compound of formula (III′) with a compound selected from R¹—O—NH₂, R²R³N—NH₂, R⁴NH(CH₂)₂₋₃SH or R⁵CH(NR⁶H)(CR⁷R⁸)₁₋₂SH.

In one embodiment, Q is selected from

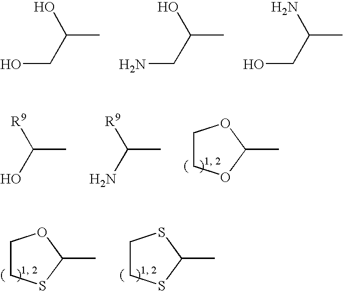

wherein R⁹ represents H, C₁₋₆alkyl, aryl, or heteroaryl, and compounds of formula (III′) takes the form of formula (III)

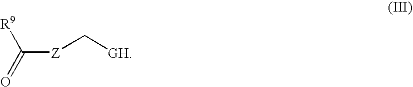

In one embodiment, the reductive alkylation of step (a) is brought about by mixing a buffered solution of GH (pH 4-8) with a solution of G-Z-C(═O)H in water or mixture of water and a dipolar solvent, followed by addition of an aqueous solution of NaCNBH₄. DMF is one example of a dipolar solvent.

Particular examples of compounds of formula (II) or (III) include

### HO—CH₂CH(OH)—CH₂—O—(CH₂)₄-GH,

### HO—CH₂CH(OH)—CH₂—O—(CH₂)₅-GH,

### HO—CH₂CH(OH)—CH₂—O—(CH₂)₆-GH,

### HO—CH₂CH(OH)—CH₂—O-(1,4-C₆H₄)CH₂-GH,

### HO—CH₂CH(OH)—CH₂—O-(1,3-C₆H₄)CH₂-GH,

### O═CH—CH₂—O—(CH₂)₄-GH,

### O═CH—CH₂—O—(CH₂)₅-GH,

### O═CH—CH₂—O—(CH₂)₆-GH,

### O═CH—CH₂—O-(1,4-C₆H₄)CH₂-GH, and

### O═CH—CH₂—O-(1,3-C₆H₄)CH₂-GH.

In one embodiment, the invention relates to a method for preparing compounds according to formula (I) or (I″), the method comprising the steps of

(a′) oxidation of the N-terminal serine extended GH of formula (IV)

Ser-GH (IV)

to a glyoxylic acid derivative of formula (V)

O═CH—C(═O)-GH (V); and

(b′) condensation of said derivative of formula V with a compound selected from R¹—O—NH₂, R²R³N—NH₂, R⁴NH(CH₂)₂₋₃SH or R⁵CH(NR⁶H)(CR⁷R⁸)₁₋₂SH.

In one embodiment, the oxidation in step (a′) is brought about by mixing a buffered solution of GH (pH 7-9) with a solution of methionine (30-300 equivalents), and adding a solution of periodate, such as NaIO₄ (5-20 equivalents). The glyoxylic acid derivative obtained exists in an equilibrium between an aldehyde form and the corresponding hydrate, as illustrated below

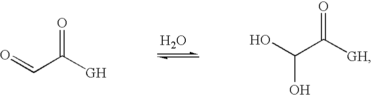

and when reference is made to compounds of formula (V), it is to be understood that reference is made to both compounds in the above equilibrium.

Particular examples of compounds of formula (IV) or (V) include

Ser-hGH, and

O═CH—C(═O)-hGH.

In one embodiment, the invention relates to methods of improving the pharmacological properties of GH, the method comprising preparing a conjugated GH as described in the above methods. In particular, improving the pharmacological properties is intended to indicate an increase in the functional in vivo half-life, the plasma in vivo half-life, the mean residence time, or an decrease in the clearance.

Compounds of formula (I) exert growth hormone activity and may as such be used in the treatment of diseases or states which will benefit from an increase in the amount of circulating growth hormone. In particular, the invention provides a method for the treatment of growth hormone deficiency (GHD); Turner Syndrome; Prader-Willi syndrome (PWS); Noonan syndrome; Down syndrome; chronic renal disease, juvenile rheumatoid arthritis; cystic fibrosis, HIV-infection in children receiving HAART treatment (HIV/HALS children); short children born short for gestational age (SGA); short stature in children born with very low birth weight (VLBW) but SGA; skeletal dysplasia; hypochondroplasia; achondroplasia; idiopathic short stature (ISS); GHD in adults; fractures in or of long bones, such as tibia, fibula, femur, humerus, radius, ulna, clavicula, matacarpea, matatarsea, and digit; fractures in or of spongious bones, such as the scull, base of hand, and base of food; patients after tendon or ligament surgery in e.g. hand, knee, or shoulder; patients having or going through distraction oteogenesis; patients after hip or discus replacement, meniscus repair, spinal fusions or prosthesis fixation, such as in the knee, hip, shoulder, elbow, wrist or jaw; patients into which osteosynthesis material, such as nails, screws and plates, have been fixed; patients with non-union or mal-union of fractures; patients after osteatomia, e.g. from tibia or 1^{st} toe; patients after graft implantation; articular cartilage degeneration in knee caused by trauma or arthritis; osteoporosis in patients with Turner syndrome; osteoporosis in men; adult patients in chronic dialysis (APCD); malnutritional associated cardiovascular disease in APCD; reversal of cachexia in APCD; cancer in APCD; chronic abstractive pulmonal disease in APCD; HIV in APCD; elderly with APCD; chronic liver disease in APCD, fatigue syndrome in APCD; Crohn's disease; impaired liver function; males with HIV infections; short bowel syndrome; central obesity; HIV-associated lipodystrophy syndrome (HALS); male infertility; patients after major elective surgery, alcohol/drug detoxification or neurological trauma; aging; frail elderly; osteo-arthritis; traumatically damaged cartilage; erectile dysfunction; fibromyalgia; memory disorders; depression; traumatic brain injury; traumatic spinal cord injury; subarachnoid haemorrhage; very low birth weight; metabolic syndrome; glucocorticoid myopathy; or short stature due to glucucorticoid treatment in children, the method comprising administering to a patient in need thereof a therapeutically effective amount of a compound according to formula (I)

In one aspect, the invention provides a method for the acceleration of the healing of muscle tissue, nervous tissue or wounds; the acceleration or improvement of blood flow to damaged tissue; or the decrease of infection rate in damaged tissue, the method comprising administration to a patient in need thereof an effective amount of a therapeutically effective amount of a compound of formula I.

In one embodiment, the invention relates to the use of compounds according to formula I in the manufacture of diseases benefiting from an increase in the growth hormone plasma level, such as the disease mentioned above.

A typical parenteral dose is in the range of 10⁻⁹ mg/kg to about 100 mg/kg body weight per administration. Typical administration doses are from about 0.0000001 to about 10 mg/kg body weight per administration. The exact dose will depend on e.g. indication, medicament, frequency and mode of administration, the sex, age and general condition of the subject to be treated, the nature and the severity of the disease or condition to be treated, the desired effect of the treatment and other factors evident to the person skilled in the art.

Typical dosing frequencies are twice daily, once daily, bi-daily, twice weekly, once weekly or with even longer dosing intervals. Due to the prolonged half-lifes of the fusion proteins of the present invention, a dosing regime with long dosing intervals, such as twice weekly, once weekly or with even longer dosing intervals is a particular embodiment of the invention.

Many diseases are treated using more than one medicament in the treatment, either concomitantly administered or sequentially administered. It is therefore within the scope of the present invention to use compounds of formula (I) in therapeutic methods for the treatment of one of the above mentioned diseases in combination with one or more other therapeutically active compound normally used in the treatment said diseases. By analogy, it is also within the scope of the present invention to use compounds of formula (I) in combination with other therapeutically active compounds normally used in the treatment of one of the above mentioned diseases in the manufacture of a medicament for said disease.

The GH and in particular Ser-GH may be prepared in a number of different ways, such as e.g. synthesis using standard protein synthesis techniques. In a particular embodiment, however, the GH or Ser-GH is expressed in a suitable host after incorporation of a suitable nucleic acid construct into said host.

As used herein the term “nucleic acid construct” is intended to indicate any nucleic acid molecule of cDNA, genomic DNA, synthetic DNA or RNA origin. The term “construct” is intended to indicate a nucleic acid segment which may be single- or double-stranded, and which may be based on a complete or partial nucleotide sequence encoding a protein of interest. The construct may optionally contain other nucleic acid segments.

The nucleic acid construct of the invention encoding the protein of the invention may suitably be of genomic or cDNA origin, for instance obtained by preparing a genomic or cDNA library and screening for DNA sequences coding for all or part of the protein by hybridization using synthetic oligonucleotide probes in accordance with standard techniques (cf. J. Sambrook, E. F. Fritsch, and T. Maniatus, 1989, Molecular Cloning, A Laboratory Manual, 2d edition, Cold Spring Harbor, N.Y.). For the present purpose, the DNA sequence encoding the GH is preferably of human origin, i.e. derived from a human genomic DNA or cDNA library. In particular, the DNA sequence may be of human origin, e.g. cDNA from a particular human organ or cell type or a gene derived from human genomic DNA.

The nucleic acid construct of the invention encoding the GH or Ser-GH may also be prepared synthetically by established standard methods, e.g. the phosphoamidite method described by Beaucage and Caruthers, Tetrahedron Letters 22 (1981), 1859-1869, or the method described by Matthes et al., EMBO Journal 3 (1984), 801-805. According to the phosphoamidite method, oligonucleotides are synthesized, e.g. in an automatic DNA synthesizer, purified, annealed, ligated and cloned in suitable vectors.

Furthermore, the nucleic acid construct may be of mixed synthetic and genomic, mixed synthetic and cDNA or mixed genomic and cDNA origin prepared by ligating fragments of synthetic, genomic or cDNA origin (as appropriate), the fragments corresponding to various parts of the entire nucleic acid construct, in accordance with standard techniques.

The nucleic acid construct may also be prepared by polymerase chain reaction using specific primers, for instance as described in U.S. Pat. No. 4,683,202 or Saiki et al., Science 239 (1988), 487-491.

In one embodiment, the nucleic acid construct is a DNA construct which term will be used exclusively in the following.

In one embodiment, the present invention relates to a recombinant vector comprising a DNA construct of the invention. The recombinant vector into which the DNA construct of the invention is inserted may be any vector which may conveniently be subjected to recombinant DNA procedures, and the choice of vector will often depend on the host cell into which it is to be introduced. Thus, the vector may be an autonomously replicating vector, i.e. a vector which exists as an extrachromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated.

The vector is preferably an expression vector in which the DNA sequence encoding the protein of the invention is operably linked to additional segments required for transcription of the DNA. In general, the expression vector is derived from plasmid or viral DNA, or may contain elements of both. The term, “operably linked” indicates that the segments are arranged so that they function in concert for their intended purposes, e.g. transcription initiates in a promoter and proceeds through the DNA sequence coding for the protein.

The promoter may be any DNA sequence which shows transcriptional activity in the host cell of choice and may be derived from genes encoding proteins either homologous or heterologous to the host cell.

Examples of suitable promoters for directing the transcription of the DNA encoding the GH in mammalian cells are the SV40 promoter (Subramani et al., Mol. Cell. Biol. 1 (1981), 854-864), the MT-1 (metallothionein gene) promoter (Palmiter et al., Science 222 (1983), 809-814) or the adenovirus 2 major late promoter.

An example of a suitable promoter for use in insect cells is the polyhedrin promoter (U.S. Pat. No. 4,745,051; Vasuvedan et al., FEBS Lett. 311, (1992) 7-11), the P10 promoter (J. M. Vlak et al., J. Gen. Virology 69, 1988, pp. 765-776), the Autographa californica polyhedrosis virus basic protein promoter (EP 397 485), the baculovirus immediate early gene 1 promoter (U.S. Pat. No. 5,155,037; U.S. Pat. No. 5,162,222), or the baculovirus 39K delayed-early gene promoter (U.S. Pat. No. 5,155,037; U.S. Pat. No. 5,162,222).

Examples of suitable promoters for use in yeast host cells include promoters from yeast glycolytic genes (Hitzeman et al., J. Biol. Chem. 255 (1980), 12073-12080; Alber and Kawasaki, J. Mol. Appl. Gen. 1 (1982), 419-434) or alcohol dehydrogenase genes (Young et al., in Genetic Engineering of Microorganisms for Chemicals (Hollaender et al, eds.), Plenum Press, New York, 1982), or the TPI1 (U.S. Pat. No. 4,599,311) or ADH2-4-c (Russell et al., Nature 304 (1983), 652-654) promoters.

Examples of suitable promoters for use in filamentous fungus host cells are, for instance, the ADH3 promoter (McKnight et al., The EMBO J. 4 (1985), 2093-2099) or the tpiA promoter. Examples of other useful promoters are those derived from the gene encoding A. oryzae TAKA amylase, Rhizomucor miehei aspartic proteinase, A. niger neutral α-amylase, A. niger acid stable α-amylase, A. niger or A. awamori glucoamylase (gluA), Rhizomucor miehei lipase, A. oryzae alkaline protease, A. oryzae triose phosphate isomerase or A. nidulans acetamidase. Preferred are the TAKA-amylase and gluA promoters.

Examples of suitable promoters for use in bacterial host cells include the promoter of the Bacillus stearothermophilus maltogenic amylase gene, the Bacillus licheniformis alpha-amylase gene, the Bacillus amyloliquefaciens BAN amylase gene, the Bacillus subtilis alkaline protease gen, or the Bacillus pumilus xylosidase gene, or by the phage Lambda P_{R} or P_{L} promoters or the E. coli lac, trp or tac promoters.

The DNA sequence encoding GH or Ser-GH may also, if necessary, be operably connected to a suitable terminator, such as the human growth hormone terminator (Palmiter et al., M. cit.) or (for fungal hosts) the TPI1 (Alber and Kawasaki, M. cit.) or ADH3 (McKnight et al., op. cit.) terminators. The vector may further comprise elements such as polyadenylation signals (e.g. from SV40 or the adenovirus 5 Elb region), transcriptional enhancer sequences (e.g. the SV40 enhancer) and translational enhancer sequences (e.g. the ones encoding adenovirus VA RNAs).

The recombinant vector of the invention may further comprise a DNA sequence enabling the vector to replicate in the host cell in question. An example of such a sequence (when the host cell is a mammalian cell) is the SV40 origin of replication.

When the host cell is a yeast cell, suitable sequences enabling the vector to replicate are the yeast plasmid 2μ replication genes REP 1-3 and origin of replication.

When the host cell is a bacterial cell, sequences enabling the vector to replicate are DNA polymerase III complex encoding genes and origin of replication.

The vector may also comprise a selectable marker, e.g. a gene the product of which complements a defect in the host cell, such as the gene coding for dihydrofolate reductase (DHFR) or the Schizosaccharomyces pombe TPI gene (described by P. R. Russell, Gene 40, 1985, pp. 125-130), or one which confers resistance to a drug, e.g. ampicillin, kanamycin, tetracyclin, chloramphenicol, neomycin, hygromycin or methotrexate. For filamentous fungi, selectable markers include amdS, pyrG, argB, niaD and sC.

To direct GH into the secretory pathway of the host cells, a secretory signal sequence (also known as a leader sequence, prepro sequence or pre sequence) may be provided in the recombinant vector. The secretory signal sequence is joined to the DNA sequence encoding the protein in the correct reading frame. Secretory signal sequences are commonly positioned 5′ to the DNA sequence encoding the protein. The secretory signal sequence may be that which is normally associated with the GH or may be from a gene encoding another secreted protein.

For secretion from yeast cells, the secretory signal sequence may encode any signal peptide which ensures efficient direction of the expressed protein into the secretory pathway of the cell. The signal peptide may be naturally occurring signal peptide, or a functional part thereof, or it may be a synthetic peptide. Suitable signal peptides have been found to be the α-factor signal peptide (cf. U.S. Pat. No. 4,870,008), the signal peptide of mouse salivary amylase (cf. O. Hagenbuchle et al., Nature 289, 1981, pp. 643-646), a modified carboxypeptidase signal peptide (cf. L. A. Valls et al., Cell 48, 1987, pp. 887-897), the yeast BAR1 signal peptide (cf. WO 87/02670), or the yeast aspartic protease 3 (YAP3) signal peptide (cf. M. Egel-Mitani et al., Yeast 6, 1990, pp. 127-137).

For efficient secretion in yeast, a sequence encoding a leader peptide may also be inserted downstream of the signal sequence and uptream of the DNA sequence encoding the protein. The function of the leader peptide is to allow the expressed protein to be directed from the endoplasmic reticulum to the Golgi apparatus and further to a secretory vesicle for secretion into the culture medium (i.e. exportation of the protein across the cell wall or at least through the cellular membrane into the periplasmic space of the yeast cell). The leader peptide may be the yeast α-factor leader (the use of which is described in e.g. U.S. Pat. No. 4,546,082, EP 16 201, EP 123 294, EP 123 544 and EP 163 529). Alternatively, the leader peptide may be a synthetic leader peptide, which is to say a leader peptide not found in nature. Synthetic leader peptides may, for instance, be constructed as described in WO 89/02463 or WO 92/11378.

For use in filamentous fungi, the signal peptide may conveniently be derived from a gene encoding an Aspergillus sp. amylase or glucoamylase, a gene encoding a Rhizomucor miehei lipase or protease or a Humicola lanuginosa lipase. The signal peptide is preferably derived from a gene encoding A. oryzae TAKA amylase, A. niger neutral α-amylase, A. niger acid-stable amylase, or A. niger glucoamylase.

For use in insect cells, the signal peptide may conveniently be derived from an insect gene (cf. WO 90/05783), such as the lepidopteran Manduca sexta adipokinetic hormone precursor signal peptide (cf. U.S. Pat. No. 5,023,328).

The procedures used to ligate the DNA sequences coding for the present protein, the promoter and optionally the terminator and/or secretory signal sequence, respectively, and to insert them into suitable vectors containing the information necessary for replication, are well known to persons skilled in the art (cf., for instance, Sambrook et al., op. cit.).

The host cell into which the DNA construct or the recombinant vector of the invention is introduced may be any cell which is capable of producing the present protein and includes bacteria, yeast, fungi and higher eukaryotic cells.

Examples of bacterial host cells which, on cultivation, are capable of producing GH are grampositive bacteria such as strains of Bacillus, such as strains of B. subtilis, B. licheniformis, B. lentus, B. brevis, B. stearothermophilus, B. alkalophilus, B. amyloliquefaciens, B. coagulans, B. circulans, B. lautus, B. megatherium or B. thuringiensis, or strains of Streptomyces, such as S. lividans or S. murinus, or gram negative bacteria such as Echerichia coli. The transformation of the bacteria may be effected by protoplast transformation or by using competent cells in a manner known per se (cf. Sambrook et al., supra).

When expressing a protein in bacteria such as E. coli, the protein may be retained in the cytoplasm, typically as insoluble granules (known as inclusion bodies), or may be directed to the periplasmic space by a bacterial secretion sequence. In the former case, the cells are lysed and the granules are recovered and denatured after which the protein is refolded by diluting the denaturing agent. In the latter case, the protein may be recovered from the periplasmic space by disrupting the cells, e.g. by sonication or osmotic shock, to release the contents of the periplasmic space and recovering the protein.

Examples of suitable mammalian cell lines are the COS (ATCC CRL 1650), BHK (ATCC CRL 1632, ATCC CCL 10), CHL (ATCC CCL39) or CHO (ATCC CCL 61) cell lines. Methods of transfecting mammalian cells and expressing DNA sequences introduced in the cells are described in e.g. Kaufman and Sharp, J. Mol. Biol. 159 (1982), 601-621; Southern and Berg, J. Mol. Appl. Genet. 1 (1982), 327-341; Loyter et al., Proc. Natl. Acad. Sci. USA 79 (1982), 422-426; Wigler et al., Cell 14 (1978), 725; Corsaro and Pearson, Somatic Cell Genetics 7 (1981), 603, Graham and van der Eb, Virology 52 (1973), 456; and Neumann et al., EMBO J. 1 (1982), 841-845.

Examples of suitable yeasts cells include cells of Saccharomyces spp. or Schizosaccharomyces spp., in particular strains of Saccharomyces cerevisiae or Saccharomyces kluyveri. Methods for transforming yeast cells with heterologous DNA and producing heterologous proteins therefrom are described, e.g. in U.S. Pat. No. 4,599,311, U.S. Pat. No. 4,931,373, U.S. Pat. Nos. 4,870,008, 5,037,743, and U.S. Pat. No. 4,845,075, all of which are hereby incorporated by reference. Transformed cells are selected by a phenotype determined by a selectable marker, commonly drug resistance or the ability to grow in the absence of a particular nutrient, e.g. leucine. A preferred vector for use in yeast is the POT1 vector disclosed in U.S. Pat. No. 4,931,373. The DNA sequence encoding a protein of the invention may be preceded by a signal sequence and optionally a leader sequence, e.g. as described above. Further examples of suitable yeast cells are strains of Kluyveromyces, such as K. lactis, Hansenula, e.g. H. polymorpha, or Pichia, e.g. P. pastoris (cf. Gleeson et al., J. Gen. Microbiol. 132, 1986, pp. 3459-3465; U.S. Pat. No. 4,882,279).

Examples of other fungal cells are cells of filamentous fungi, e.g. Aspergillus spp., Neurospora spp., Fusarium spp. or Trichoderma spp., in particular strains of A. oryzae, A. nidulans or A. niger. The use of Aspergillus spp. for the expression of proteins is described in, e.g., EP 272 277 and EP 230 023. The transformation of F. oxysporum may, for instance, be carried out as described by Malardier et al., 1989, Gene 78: 147-156.

When a filamentous fungus is used as the host cell, it may be transformed with the DNA construct of the invention, conveniently by integrating the DNA construct in the host chromosome to obtain a recombinant host cell. This integration is generally considered to be an advantage as the DNA sequence is more likely to be stably maintained in the cell. Integration of the DNA constructs into the host chromosome may be performed according to conventional methods, e.g. by homologous or heterologous recombination.

Transformation of insect cells and production of heterologous proteins therein may be performed as described in U.S. Pat. No. 4,745,051; U.S. Pat. No. 4,879,236; U.S. Pat. Nos. 5,155,037; 5,162,222; EP 397,485) all of which are incorporated herein by reference. The insect cell line used as the host may suitably be a Lepidopteracell line, such as Spodoptera frugiperda cells or Trichoplusia ni cells (cf. U.S. Pat. No. 5,077,214). Culture conditions may suitably be as described in, for instance, WO 89/01029 or WO 89/01028, or any of the aforementioned references.

The transformed or transfected host cell described above is then cultured in a suitable nutrient medium under conditions permitting the expression of the present protein, after which the resulting protein is recovered from the culture.

The medium used to culture the cells may be any conventional medium suitable for growing the host cells, such as minimal or complex media containing appropriate supplements. Suitable media are available from commercial suppliers or may be prepared according to published recipes (e.g. in catalogues of the American Type Culture Collection). The protein produced by the cells may then be recovered from the culture medium by conventional procedures including separating the host cells from the medium by centrifugation or filtration, precipitating the proteinaceous components of the supernatant or filtrate by means of a salt, e.g. ammonium sulphate, purification by a variety of chromatographic procedures, e.g. ion exchange chromatography, gelfiltration chromatography, affinity chromatography, or the like, dependent on the specific protein in question.

### Pharmaceutical Compositions

Another purpose is to provide a pharmaceutical composition comprising a conjugated GH of the present invention which is present in a concentration from 10⁻¹⁵ mg/ml to 200 mg/ml, such as e.g. 10⁻¹⁰ mg/ml to 5 mg/ml and wherein said composition has a pH from 2.0 to 10.0. The composition may further comprise a buffer system, preservative(s), tonicity agent(s), chelating agent(s), stabilizers and surfactants. In one embodiment of the invention the pharmaceutical composition is an aqueous composition, i.e. composition comprising water. Such composition is typically a solution or a suspension. In a further embodiment of the invention the pharmaceutical composition is an aqueous solution. The term “aqueous composition” is defined as a composition comprising at least 50% w/w water. Likewise, the term “aqueous solution” is defined as a solution comprising at least 50% w/w water, and the term “aqueous suspension” is defined as a suspension comprising at least 50% w/w water.

In another embodiment the pharmaceutical composition is a freeze-dried composition, whereto the physician or the patient adds solvents and/or diluents prior to use.

In another embodiment the pharmaceutical composition is a dried composition (e.g. freeze-dried or spray-dried) ready for use without any prior dissolution.

In a further aspect the invention relates to a pharmaceutical composition comprising an aqueous solution of a GH conjugate, and a buffer, wherein said GH conjugate is present in a concentration from 0.1-100 mg/ml or above, and wherein said composition has a pH from about 2.0 to about 10.0.

In a another embodiment of the invention the pH of the composition is selected from the list consisting of 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, and 10.0.

In a further embodiment of the invention the buffer is selected from the group consisting of sodium acetate, sodium carbonate, citrate, glycylglycine, histidine, glycine, lysine, arginine, sodium dihydrogen phosphate, disodium hydrogen phosphate, sodium phosphate, and tris(hydroxymethyl)-aminomethan, bicine, tricine, malic acid, succinate, maleic acid, fumaric acid, tartaric acid, aspartic acid or mixtures thereof. Each one of these specific buffers constitutes an alternative embodiment of the invention.

In a further embodiment of the invention the composition further comprises a pharmaceutically acceptable preservative. In a further embodiment of the invention the preservative is selected from the group consisting of phenol, o-cresol, m-cresol, p-cresol, methyl p-hydroxybenzoate, propyl p-hydroxybenzoate, 2-phenoxyethanol, butyl p-hydroxybenzoate, 2-phenylethanol, benzyl alcohol, chlorobutanol, and thiomerosal, bronopol, benzoic acid, imidurea, chlorohexidine, sodium dehydroacetate, chlorocresol, ethyl p-hydroxybenzoate, benzethonium chloride, chlorphenesine (3p-chlorphenoxypropane-1,2-diol) or mixtures thereof. In a further embodiment of the invention the preservative is present in a concentration from 0.1 mg/ml to 20 mg/ml. In a further embodiment of the invention the preservative is present in a concentration from 0.1 mg/ml to 5 mg/ml. In a further embodiment of the invention the preservative is present in a concentration from 5 mg/ml to 10 mg/ml. In a further embodiment of the invention the preservative is present in a concentration from 10 mg/ml to 20 mg/ml. Each one of these specific preservatives constitutes an alternative embodiment of the invention. The use of a preservative in pharmaceutical compositions is well-known to the skilled person. For convenience reference is made to Remington: The Science and Practice of Pharmacy, 20^{th} edition, 2000.

In a further embodiment of the invention the composition further comprises an isotonic agent. In a further embodiment of the invention the isotonic agent is selected from the group consisting of a salt (e.g. sodium chloride), a sugar or sugar alcohol, an amino acid (e.g. L-glycine, L-histidine, arginine, lysine, isoleucine, aspartic acid, tryptophan, threonine), an alditol (e.g. glycerol (glycerine), 1,2-propanediol (propyleneglycol), 1,3-propanediol, 1,3-butanediol) polyethyleneglycol (e.g. PEG400), or mixtures thereof. Any sugar such as mono-, di-, or polysaccharides, or water-soluble glucans, including for example fructose, glucose, mannose, sorbose, xylose, maltose, lactose, sucrose, trehalose, dextran, pullulan, dextrin, cyclodextrin, soluble starch, hydroxyethyl starch and carboxymethylcellulose-Na may be used. In one embodiment the sugar additive is sucrose. Sugar alcohol is defined as a C4-C8 hydrocarbon having at least one —OH group and includes, for example, mannitol, sorbitol, inositol, galactitol, dulcitol, xylitol, and arabitol. In one embodiment the sugar alcohol additive is mannitol. The sugars or sugar alcohols mentioned above may be used individually or in combination. There is no fixed limit to the amount used, as long as the sugar or sugar alcohol is soluble in the liquid preparation and does not adversely effect the stabilizing effects obtained using the methods of the invention. In one embodiment, the sugar or sugar alcohol concentration is between about 1 mg/ml and about 150 mg/ml. In a further embodiment of the invention the isotonic agent is present in a concentration from 1 mg/ml to 50 mg/ml. In a further embodiment of the invention the isotonic agent is present in a concentration from 1 mg/ml to 7 mg/ml. In a further embodiment of the invention the isotonic agent is present in a concentration from 8 mg/ml to 24 mg/ml. In a further embodiment of the invention the isotonic agent is present in a concentration from 25 mg/ml to 50 mg/ml. Each one of these specific isotonic agents constitutes an alternative embodiment of the invention. The use of an isotonic agent in pharmaceutical compositions is well-known to the skilled person. For convenience reference is made to Remington: The Science and Practice of Pharmacy, 20^{th} edition, 2000.

In a further embodiment of the invention the composition further comprises a chelating agent. In a further embodiment of the invention the chelating agent is selected from salts of ethylenediaminetetraacetic acid (EDTA), citric acid, and aspartic acid, and mixtures thereof. In a further embodiment of the invention the chelating agent is present in a concentration from 0.1 mg/ml to 5 mg/ml. In a further embodiment of the invention the chelating agent is present in a concentration from 0.1 mg/ml to 2 mg/ml. In a further embodiment of the invention the chelating agent is present in a concentration from 2 mg/ml to 5 mg/ml. Each one of these specific chelating agents constitutes an alternative embodiment of the invention. The use of a chelating agent in pharmaceutical compositions is well-known to the skilled person. For convenience reference is made to Remington: The Science and Practice of Pharmacy, 20^{th} edition, 2000.

In a further embodiment of the invention the composition further comprises a stabilizer. The use of a stabilizer in pharmaceutical compositions is well-known to the skilled person. For convenience reference is made to Remington: The Science and Practice of Pharmacy, 20^{th} edition, 2000.

More particularly, compositions of the invention are stabilized liquid pharmaceutical compositions whose therapeutically active components include a protein that possibly exhibits aggregate formation during storage in liquid pharmaceutical compositions. By “aggregate formation” is intended a physical interaction between the protein molecules that results in formation of oligomers, which may remain soluble, or large visible aggregates that precipitate from the solution. By “during storage” is intended a liquid pharmaceutical composition or composition once prepared, is not immediately administered to a subject. Rather, following preparation, it is packaged for storage, either in a liquid form, in a frozen state, or in a dried form for later reconstitution into a liquid form or other form suitable for administration to a subject. By “dried form” is intended the liquid pharmaceutical composition or composition is dried either by freeze drying (i.e., lyophilization; see, for example, Williams and Polli (1984) J. Parenteral Sci. Technol. 38:48-59), spray drying (see Masters (1991) in Spray-Drying Handbook (5th ed; Longman Scientific and Technical, Essez, U.K.), pp. 491-676; Broadhead et al. (1992) Drug Devel. Ind. Pharm. 18:1169-1206; and Mumenthaler et al. (1994) Pharm. Res. 11:12-20), or air drying (Carpenter and Crowe (1988) Cryobiology 25:459-470; and Roser (1991) Biopharm. 4:47-53). Aggregate formation by a protein during storage of a liquid pharmaceutical composition can adversely affect biological activity of that protein, resulting in loss of therapeutic efficacy of the pharmaceutical composition. Furthermore, aggregate formation may cause other problems such as blockage of tubing, membranes, or pumps when the protein-containing pharmaceutical composition is administered using an infusion system.

The pharmaceutical compositions of the invention may further comprise an amount of an amino acid base sufficient to decrease aggregate formation by the protein during storage of the composition. By “amino acid base” is intended an amino acid or a combination of amino acids, where any given amino acid is present either in its free base form or in its salt form. Where a combination of amino acids is used, all of the amino acids may be present in their free base forms, all may be present in their salt forms, or some may be present in their free base forms while others are present in their salt forms. In one embodiment, amino acids to use in preparing the compositions of the invention are those carrying a charged side chain, such as arginine, lysine, aspartic acid, and glutamic acid. Any stereoisomer (i.e., L or D isomer, or mixtures thereof) of a particular amino acid (methionine, histidine, arginine, lysine, isoleucine, aspartic acid, tryptophan, threonine and mixtures thereof) or combinations of these stereoisomers or glycine or an organic base such as but not limited to imidazole, may be present in the pharmaceutical compositions of the invention so long as the particular amino acid or organic base is present either in its free base form or its salt form. In one embodiment the L-stereoisomer of an amino acid is used. In one embodiment the L-stereoisomer is used. Compositions of the invention may also be formulated with analogues of these amino acids. By “amino acid analogue” is intended a derivative of the naturally occurring amino acid that brings about the desired effect of decreasing aggregate formation by the protein during storage of the liquid pharmaceutical compositions of the invention. Suitable arginine analogues include, for example, aminoguanidine, ornithine and N-monoethyl L-arginine, suitable methionine analogues include ethionine and buthionine and suitable cysteine analogues include S-methyl-L cysteine. As with the other amino acids, the amino acid analogues are incorporated into the compositions in either their free base form or their salt form. In a further embodiment of the invention the amino acids or amino acid analogues are used in a concentration, which is sufficient to prevent or delay aggregation of the protein.

In a further embodiment of the invention methionine (or other sulphuric amino acids or amino acid analogous) may be added to inhibit oxidation of methionine residues to methionine sulfoxide when the protein acting as the therapeutic agent is a protein comprising at least one methionine residue susceptible to such oxidation. By “inhibit” is intended minimal accumulation of methionine oxidized species over time. Inhibiting methionine oxidation results in greater retention of the protein in its proper molecular form. Any stereoisomer of methionine (L or D isomer) or any combinations thereof can be used. The amount to be added should be an amount sufficient to inhibit oxidation of the methionine residues such that the amount of methionine sulfoxide is acceptable to regulatory agencies. Typically, this means that the composition contains no more than about 10% to about 30% methionine sulfoxide. Generally, this can be obtained by adding methionine such that the ratio of methionine added to methionine residues ranges from about 1:1 to about 1000:1, such as 10:1 to about 100:1.

In a further embodiment of the invention the composition further comprises a stabilizer selected from the group of high molecular weight polymers or low molecular compounds. In a further embodiment of the invention the stabilizer is selected from polyethylene glycol (e.g. PEG 3350), polyvinyl alcohol (PVA), polyvinylpyrrolidone, carboxy-/hydroxycellulose or derivates thereof (e.g. HPC, HPC-SL, HPC-L and HPMC), cyclodextrins, sulphur-containing substances as monothioglycerol, thioglycolic acid and 2-methylthioethanol, and different salts (e.g. sodium chloride). Each one of these specific stabilizers constitutes an alternative embodiment of the invention.

The pharmaceutical compositions may also comprise additional stabilizing agents, which further enhance stability of a therapeutically active protein therein. Stabilizing agents of particular interest to the present invention include, but are not limited to, methionine and EDTA, which protect the protein against methionine oxidation, and a nonionic surfactant, which protects the protein against aggregation associated with freeze-thawing or mechanical shearing.

In a further embodiment of the invention the composition further comprises a surfactant. In a further embodiment of the invention the surfactant is selected from a detergent, ethoxylated castor oil, polyglycolyzed glycerides, acetylated monoglycerides, sorbitan fatty acid esters, polyoxypropylene-polyoxyethylene block polymers (eg. poloxamers such as Pluronic® F68, poloxamer 188 and 407, Triton X-100), polyoxyethylene sorbitan fatty acid esters, polyoxyethylene and polyethylene derivatives such as alkylated and alkoxylated derivatives (tweens, e.g. Tween-20, Tween-40, Tween-80 and Brij-35), monoglycerides or ethoxylated derivatives thereof, diglycerides or polyoxyethylene derivatives thereof, alcohols, glycerol, lectins and phospholipids (eg. phosphatidyl serine, phosphatidyl choline, phosphatidyl ethanolamine, phosphatidyl inositol, diphosphatidyl glycerol and sphingomyelin), derivates of phospholipids (eg. dipalmitoyl phosphatidic acid) and lysophospholipids (eg. palmitoyl lysophosphatidyl-L-serine and 1-acyl-sn-glycero-3-phosphate esters of ethanolamine, choline, serine or threonine) and alkyl, alkoxyl(alkyl ester), alkoxy(alkyl ether)-derivatives of lysophosphatidyl and phosphatidylcholines, e.g. lauroyl and myristoyl derivatives of lysophosphatidylcholine, dipalmitoylphosphatidylcholine, and modifications of the polar head group, that is cholines, ethanolamines, phosphatidic acid, serines, threonines, glycerol, inositol, and the positively charged DODAC, DOTMA, DCP, BISHOP, lysophosphatidylserine and lysophosphatidylthreonine, and glycerophospholipids (eg. cephalins), glyceroglycolipids (eg. galactopyransoide), sphingoglycolipids (eg. ceramides, gangliosides), dodecylphosphocholine, hen egg lysolecithin, fusidic acid derivatives—(e.g. sodium tauro-dihydrofusidate etc.), long-chain fatty acids and salts thereof C₆-C₁₂ (eg. oleic acid and caprylic acid), acylcarnitines and derivatives, N^{α}-acylated derivatives of lysine, arginine or histidine, or side-chain acylated derivatives of lysine or arginine, N^{α}-acylated derivatives of dipeptides comprising any combination of lysine, arginine or histidine and a neutral or acidic amino acid, N′-acylated derivative of a tripeptide comprising any combination of a neutral amino acid and two charged amino acids, DSS (docusate sodium, CAS registry no [577-11-7]), docusate calcium, CAS registry no [128-49-4]), docusate potassium, CAS registry no [7491-09-0]), SDS (sodium dodecyl sulphate or sodium lauryl sulphate), sodium caprylate, cholic acid or derivatives thereof, bile acids and salts thereof and glycine or taurine conjugates, ursodeoxycholic acid, sodium cholate, sodium deoxycholate, sodium taurocholate, sodium glycocholate, N-Hexadecyl-N,N-dimethyl-3-ammonio-1-propanesulfonate, anionic (alkyl-aryl-sulphonates) monovalent surfactants, zwitterionic surfactants (e.g. N-alkyl-N,N-dimethylammonio-1-propanesulfonates, 3-cholamido-1-propyldimethylammonio-1-propanesulfonate, cationic surfactants (quaternary ammonium bases) (e.g. cetyl-trimethylammonium bromide, cetylpyridinium chloride), non-ionic surfactants (eg. Dodecyl β-D-glucopyranoside), poloxamines (eg. Tetronic's), which are tetrafunctional block copolymers derived from sequential addition of propylene oxide and ethylene oxide to ethylenediamine, or the surfactant may be selected from the group of imidazoline derivatives, or mixtures thereof. Each one of these specific surfactants constitutes an alternative embodiment of the invention.

The use of a surfactant in pharmaceutical compositions is well-known to the skilled person. For convenience reference is made to Remington: The Science and Practice of Pharmacy, 20^{th} edition, 2000.

It is possible that other ingredients may be present in the pharmaceutical composition of the present invention. Such additional ingredients may include wetting agents, emulsifiers, antioxidants, bulking agents, tonicity modifiers, chelating agents, metal ions, oleaginous vehicles, proteins (e.g., human serum albumin, gelatine or proteins) and a zwitterion (e.g., an amino acid such as betaine, taurine, arginine, glycine, lysine and histidine). Such additional ingredients, of course, should not adversely affect the overall stability of the pharmaceutical composition of the present invention.

Pharmaceutical compositions containing a GH conjugate according to the present invention may be administered to a patient in need of such treatment at several sites, for example, at topical sites, for example, skin and mucosal sites, at sites which bypass absorption, for example, administration in an artery, in a vein, in the heart, and at sites which involve absorption, for example, administration in the skin, under the skin, in a muscle or in the abdomen.

Administration of pharmaceutical compositions according to the invention may be through several routes of administration, for example, lingual, sublingual, buccal, in the mouth, oral, in the stomach and intestine, nasal, pulmonary, for example, through the bronchioles and alveoli or a combination thereof, epidermal, dermal, transdermal, vaginal, rectal, ocular, for examples through the conjunctiva, uretal, and parenteral to patients in need of such a treatment.

Compositions of the current invention may be administered in several dosage forms, for example, as solutions, suspensions, emulsions, microemulsions, multiple emulsion, foams, salves, pastes, plasters, ointments, tablets, coated tablets, rinses, capsules, for example, hard gelatine capsules and soft gelatine capsules, suppositories, rectal capsules, drops, gels, sprays, powder, aerosols, inhalants, eye drops, ophthalmic ointments, ophthalmic rinses, vaginal pessaries, vaginal rings, vaginal ointments, injection solution, in situ transforming solutions, for example in situ gelling, in situ setting, in situ precipitating, in situ crystallization, infusion solution, and implants.

Compositions of the invention may further be compounded in, or attached to, for example through covalent, hydrophobic and electrostatic interactions, a drug carrier, drug delivery system and advanced drug delivery system in order to further enhance stability of the GH conjugate, increase bioavailability, increase solubility, decrease adverse effects, achieve chronotherapy well known to those skilled in the art, and increase patient compliance or any combination thereof. Examples of carriers, drug delivery systems and advanced drug delivery systems include, but are not limited to, polymers, for example cellulose and derivatives, polysaccharides, for example dextran and derivatives, starch and derivatives, poly(vinyl alcohol), acrylate and methacrylate polymers, polylactic and polyglycolic acid and block co-polymers thereof, polyethylene glycols, carrier proteins, for example albumin, gels, for example, thermogelling systems, for example block co-polymeric systems well known to those skilled in the art, micelles, liposomes, microspheres, nanoparticulates, liquid crystals and dispersions thereof, L2 phase and dispersions there of, well known to those skilled in the art of phase behaviour in lipid-water systems, polymeric micelles, multiple emulsions, self-emulsifying, self-microemulsifying, cyclodextrins and derivatives thereof, and dendrimers.

Compositions of the current invention are useful in the composition of solids, semisolids, powder and solutions for pulmonary administration of GH conjugate, using, for example a metered dose inhaler, dry powder inhaler and a nebulizer, all being devices well known to those skilled in the art.

Compositions of the current invention are specifically useful in the composition of controlled, sustained, protracting, retarded, and slow release drug delivery systems. More specifically, but not limited to, compositions are useful in composition of parenteral controlled release and sustained release systems (both systems leading to a many-fold reduction in number of administrations), well known to those skilled in the art. Even more preferably, are controlled release and sustained release systems administered subcutaneous. Without limiting the scope of the invention, examples of useful controlled release system and compositions are hydrogels, oleaginous gels, liquid crystals, polymeric micelles, microspheres, nanoparticles,

Methods to produce controlled release systems useful for compositions of the current invention include, but are not limited to, crystallization, condensation, co-crystallization, precipitation, co-precipitation, emulsification, dispersion, high pressure homogenisation, encapsulation, spray drying, microencapsulating, coacervation, phase separation, solvent evaporation to produce microspheres, extrusion and supercritical fluid processes. General reference is made to Handbook of Pharmaceutical Controlled Release (Wise, D. L., ed. Marcel Dekker, New York, 2000) and Drug and the Pharmaceutical Sciences vol. 99: Protein Composition and Delivery (MacNally, E. J., ed. Marcel Dekker, New York, 2000).

Parenteral administration may be performed by subcutaneous, intramuscular, intraperitoneal or intravenous injection by means of a syringe, optionally a pen-like syringe. Alternatively, parenteral administration can be performed by means of an infusion pump. A further option is a composition which may be a solution or suspension for the administration of the GH conjugate in the form of a nasal or pulmonal spray. As a still further option, the pharmaceutical compositions containing the GH conjugate of the invention can also be adapted to transdermal administration, e.g. by needle-free injection or from a patch, optionally an iontophoretic patch, or transmucosal, e.g. buccal, administration.

The term “stabilized composition” refers to a composition with increased physical stability, increased chemical stability or increased physical and chemical stability.

The term “physical stability” of the protein composition as used herein refers to the tendency of the protein to form biologically inactive and/or insoluble aggregates of the protein as a result of exposure of the protein to thermo-mechanical stresses and/or interaction with interfaces and surfaces that are destabilizing, such as hydrophobic surfaces and interfaces. Physical stability of the aqueous protein compositions is evaluated by means of visual inspection and/or turbidity measurements after exposing the composition filled in suitable containers (e.g. cartridges or vials) to mechanical/physical stress (e.g. agitation) at different temperatures for various time periods. Visual inspection of the compositions is performed in a sharp focused light with a dark background. The turbidity of the composition is characterized by a visual score ranking the degree of turbidity for instance on a scale from 0 to 3 (a composition showing no turbidity corresponds to a visual score 0, and a composition showing visual turbidity in daylight corresponds to visual score 3). A composition is classified physical unstable with respect to protein aggregation, when it shows visual turbidity in daylight. Alternatively, the turbidity of the composition can be evaluated by simple turbidity measurements well-known to the skilled person. Physical stability of the aqueous protein compositions can also be evaluated by using a spectroscopic agent or probe of the conformational status of the protein. The probe is preferably a small molecule that preferentially binds to a non-native conformer of the protein. One example of a small molecular spectroscopic probe of protein structure is Thioflavin T. Thioflavin T is a fluorescent dye that has been widely used for the detection of amyloid fibrils. In the presence of fibrils, and perhaps other protein configurations as well, Thioflavin T gives rise to a new excitation maximum at about 450 nm and enhanced emission at about 482 nm when bound to a fibril protein form. Unbound Thioflavin T is essentially non-fluorescent at the wavelengths.

Other small molecules can be used as probes of the changes in protein structure from native to non-native states. For instance the “hydrophobic patch” probes that bind preferentially to exposed hydrophobic patches of a protein. The hydrophobic patches are generally buried within the tertiary structure of a protein in its native state, but become exposed as a protein begins to unfold or denature. Examples of these small molecular, spectroscopic probes are aromatic, hydrophobic dyes, such as anthracene, acridine, phenanthroline or the like. Other spectroscopic probes are metal-amino acid complexes, such as cobalt metal complexes of hydrophobic amino acids, such as phenylalanine, leucine, isoleucine, methionine, and valine, or the like.

The term “chemical stability” of the protein composition as used herein refers to chemical covalent changes in the protein structure leading to formation of chemical degradation products with potential less biological potency and/or potential increased immunogenic properties compared to the native protein structure. Various chemical degradation products can be formed depending on the type and nature of the native protein and the environment to which the protein is exposed. Elimination of chemical degradation can most probably not be completely avoided and increasing amounts of chemical degradation products is often seen during storage and use of the protein composition as well-known by the person skilled in the art. Most proteins are prone to deamidation, a process in which the side chain amide group in glutaminyl or asparaginyl residues is hydrolysed to form a free carboxylic acid. Other degradations pathways involves formation of high molecular weight transformation products where two or more protein molecules are covalently bound to each other through transamidation and/or disulfide interactions leading to formation of covalently bound dimer, oligomer and polymer degradation products (Stability of Protein Pharmaceuticals, Ahern. T. J. & Manning M. C., Plenum Press, New York 1992). Oxidation (of for instance methionine residues) can be mentioned as another variant of chemical degradation. The chemical stability of the protein composition can be evaluated by measuring the amount of the chemical degradation products at various time-points after exposure to different environmental conditions (the formation of degradation products can often be accelerated by for instance increasing temperature). The amount of each individual degradation product is often determined by separation of the degradation products depending on molecule size and/or charge using various chromatography techniques (e.g. SEC-HPLC and/or RP-HPLC).

Hence, as outlined above, a “stabilized composition” refers to a composition with increased physical stability, increased chemical stability or increased physical and chemical stability. In general, a composition must be stable during use and storage (in compliance with recommended use and storage conditions) until the expiration date is reached.

In one embodiment of the invention the pharmaceutical composition comprising the GH conjugate is stable for more than 6 weeks of usage and for more than 3 years of storage.

In another embodiment of the invention the pharmaceutical composition comprising the GH conjugate is stable for more than 4 weeks of usage and for more than 3 years of storage.

In a further embodiment of the invention the pharmaceutical composition comprising the GH conjugate is stable for more than 4 weeks of usage and for more than two years of storage.

In an even further embodiment of the invention the pharmaceutical composition comprising the GH conjugate is stable for more than 2 weeks of usage and for more than two years of storage.

All references, including publications, patent applications and patents, cited herein are hereby incorporated by reference to the same extent as if each reference was individually and specifically indicated to be incorporated by reference and was set forth in its entirety herein.

All headings and sub-headings are used herein for convenience only and should not be construed as limiting the invention in any way,

Any combination of the above-described elements in all possible variations thereof is encompassed by the invention unless otherwise indicated herein or otherwise clearly contradicted by context.

The terms “a” and “an” and “the” and similar referents as used in the context of describing the invention are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein. Unless otherwise stated, all exact values provided herein are representative of corresponding approximate values (e.g., all exact exemplary values provided with respect to a particular factor or measurement can be considered to also provide a corresponding approximate measurement, modified by “about,” where appropriate).

All methods described herein can be performed in any suitable order unless otherwise indicated herein or otherwise clearly contradicted by context.

The use of any and all examples, or exemplary language (e.g., “such as”) provided herein, is intended merely to better illuminate the invention and does not pose a limitation on the scope of the invention unless otherwise indicated. No language in the specification should be construed as indicating any element is essential to the practice of the invention unless as much is explicitly stated.

The citation and incorporation of patent documents herein is done for convenience only and does not reflect any view of the validity, patentability and/or enforceability of such patent documents,

The description herein of any aspect or embodiment of the invention using terms such as “comprising”, “having”, “including” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

This invention includes all modifications and equivalents of the subject matter recited in the aspects or claims presented herein to the maximum extent permitted by applicable law.

### EXAMPLES

The following abbreviations are used:
Boc: tert-butyloxycarbonyl
Bt: 1-benzotriazolyl
DBU: 1,8-diazabicyclo[5.4.0]undec-7-ene
DCM: dichloromethane, methylenechloride
Dde: 1-(4,4-dimethyl-2,6-dioxocyclohexylidene)ethyl
DIC: diisopropylcarbodiimide
DMA: N,N-dimethylacetamide
DMF: N,N-dimethyl formamide
DMSO: dimethyl sulfoxide
DMAP: 4-dimethylaminopyridine
DMPU: 1,3-dimethyltetrahydropyrimidin-2-one
EDC: N-ethyl-N′-(3-dimethylaminopropyl)carbodiimide hydrochloride
Fmoc: 9-fluorenylmethyloxycarbonyl
HBTU: 2-(1H-Benzotriazol-1-yl-)-1,1,3,3 tetramethyluronium hexafluorophosphate
HOAt: 3-hydroxy-3H-[1,2,3]triazolo[4,5-b]pyridine, 4-aza-3-hydroxybenzotriazole
HOBt: N-hydroxybenzotriazole, 1-hydroxybenzotriazole
HONSu: N-hydroxysuccinimide
NMP: N-methylpyrrolidone
HPLC: high pressure liquid chromatography
Pmc 2,2,5,7,8-pentamethylchroman-6-sulfonyl
r.t. room temperature
Su: succinimidyl
TFA: trifluoroacetic acid
TIS triisopropylsilane
Trt: trityl, triphenylmethyl
Ts: toluenesulfonyl
TSTU O-(1-succinimidyl)-N,N,N′,N′-tetramethyluronium hexafluorophosphate

NMR spectra were recorded on Bruker 300 MHz and 400 MHz instruments. HPLC-MS was performed on a Perkin Elmer instrument (API 100).

HPLC-systems from Merck-Hitachi (Hibar™ RT 250-4, Lichrosorb™ RP 18, 5.0 μm, 4.0×250 mm, gradient elution, 20% to 80% acetonitrile in water within 30 min, 1.0 ml/min, detection at 254 nm) and Waters (Symmetry™, C18, 3.5 μm, 3.0×150 mm, gradient elution, 5% to 90% acetonitrile in water within 15 min, 1.0 ml/min, detection at 214 nm) were used.

### General Method (A)

The compounds of formula (II) and (III) according to the invention may be prepared as follows:

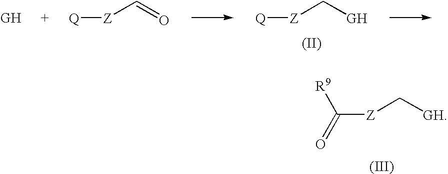

To a solution of GH in a buffer (pH 4-8) is added a solution of the aldehyde Q-Z-CHO in water or a mixture of water and a dipolar solvent, followed by addition of an aqueous solution of NaCNBH₄. The resulting mixture is shaken at room temperature for 1-24 h. Addition of a buffer (pH≧7), followed by standard purification yields the compound of formula (II). As mentioned above, Q is a functional group which can be converted into an aldehyde or ketone. The conditions required for converting compounds of formula (II) into compounds of formula (III) depend on the structure of the group Q. If Q contains hydroxyl (OH) or primary amino groups (NH₂), then a periodate oxidation as described in the General Procedure (B) can bring about said conversion. If compound of formula (II) is an acetal, aminal, hemiaminal, thioacetal, dithioacetal, or any other type of acetal, then the conversion may require treatment of compounds of formula (II) with an acid or with an oxidant. Alternatively, the condensation reaction to yield compounds of formula (I) or (I′) may also be performed directly by mixing compounds of formula (II), in which Q is an acetal-type structure, with a suitable alkoxylamine, hydrazine, or aminothiol, in the presence of an acid or a silver salt or a mercury salt, as described in the literature (Tumelty et al., J. Am. Chem. Soc. 2003, 125, 14238-14239).

### General Method (B)

The compounds of formula (V) according to the invention may be prepared as follows:

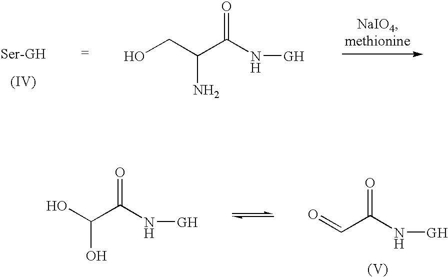

To a solution of Ser-GH in a suitable buffer (pH=7-9) a solution methionine (30-300 equivalents) in water and subsequently a solution of sodium periodate (5-20 equivalents) in water is added. The resulting solution is shaken carefully for 5-30 min, to yield a solution containing a mixture of the aldehyde and its hydrate. This product mixture can be used without further purification.

### General Method (C)

Condensation of compounds of general formula (III) or of general formula (V) with alkoxylamines, hydrazines, or aminothiols is performed by adding a solution of a suitable alkoxylamine, hydrazine, or aminothiol in water or in a mixture of water and a dipolar solvent or in a dipolar solvent alone, such as DMF, to the solution of compounds (III) or (V). The pH of the resulting mixture may range from 2.0 to 12.0, and may optionally be adjusted to a given value, for instance 3.6, 4.0, 6.0, 6.5, 10.0 or 10.5. The resulting mixture is shaken carefully for 1-200 h, and the reaction is followed by HPLC or any other, conventional analytical tool. When the reaction is finished the mixture is diluted with water, and the product purified using standard chromatographic techniques.

### Example 1

### Preparation of Ser-hGH

The Ser-hGH analogue expression plasmid was created on the basis of pNNC13 (Zbasic2mt-D4K-hGH), which expresses the wild type hGH in fusion with Zbasic domain (mvdnkfnkerrrarreirhlpnlnreqrrapirslrddpsqsanllaeakklnraqapkyrggsddddksfptiplsrlfdnamlrah rlhqlafdtyqefeeayipkeqkysflqnpqtslcfsesiptpsnreetqqksnlellrisllliqswlepvqfrsvfanslvygasdsnvy dllkdleegiqtlmgrledgsprtgqifkqtyskfdtnshnddallknygllycfrkdmdkvetflrivqcrsvegscgf). Additional Ser was inserted in front of Phe, the first amino acid of mature hGH, by QuikChange® XL Site-Directed Mutagenesis Kit from Stratagene with a pair of primes: 5′ end: pNNC13 Ser-F

5′-GGATCAGACGACGACGACAAAagcTTCCCAACCATTCCCTTATCC-3′ and 3′ end: pNNC13 Ser-R

### 5′-GGATAAGGGAATGGTTGGGAAgctTTTGTCGTCGTCGTCTGATCC-3′.

E. coli BL21 (DE3) was transformed by pET11a-Zbasic2mt-D4K-Ser-hGH. Single colony was inoculated into 100 ml LB media with 100 μg/ml Amp and grew at 37° C. When OD600 reached 0.6, the cell culture temperature was reduced to 30° C., and the cells were induced with 1 mM IPTG for 4 hours at 30 degree. The bacteria cells were harvested by centrifugation at 3000 g for 15 minutes (Eppendorf centrifuge 5810R). The cell pellet was re-suspended in cell lysis buffer (25 mM Na₂HPO₄ 25 mM NaH₂PO₄ pH 7, 5 mM EDTA, 0.1% Triton X-100), and the cells were disrupted by cell disruption at 30 kpsi (Constant Cell Disruption Systems). The lysate was clarified by centrifugation at 10000 g for 30 minutes. The supernatant was saved and used for purification, while the pellet was discarded.

Zbasic2mt-D4K-Ser-hGH was purified on SP-Sepharose using a step gradient elution (buffer A: 25 mM Na₂HPO₄ 25 mM NaH₂PO₄ pH 7; buffer B: 25 mM Na₂HPO₄ 25 mM NaH₂PO₄ pH 7, 1 M NaCl). The protein was subsequently cleaved using Enteropeptidase for the release of Ser-hGH. Ser-hGH was further purified on a Butyl Sepharose 4FF column to separate the product from the Zbasic2mt-D4K domain and Enteropeptidase (buffer A: 100 mM Hepes pH 7.5; buffer B: 100 mM Hepes pH 7.5, 2 M NaCl, a linear gradient was used). The final product of Ser-hGH was buffer exchanged and lyophilized from 50 mM NH₄HCO₃, pH 7.8.

### Example 2

### 1-[2-(O-(2-(N-Hexadecanoylpiperidin-4-yl)ethyl)oximino)acetyl]-hGH

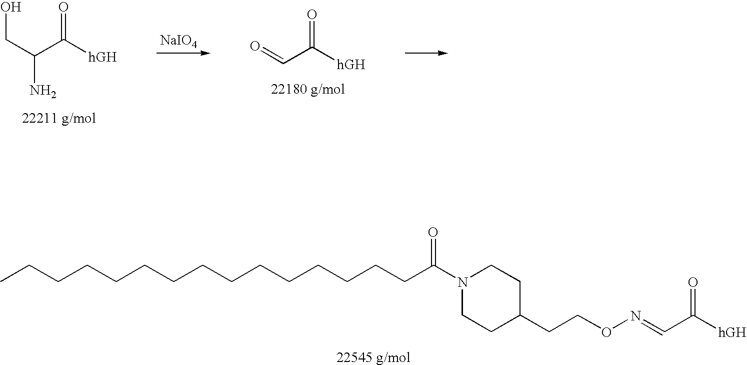

The following solutions were prepared:

Buffer: 25 μl (188 μmol) triethanolamine (Mwt: 149.21, d: 1.12) in water (10 ml)
Methionine (Mwt: 149.21): 20 mg (134 μmol) in water (1.0 ml)
NaIO₄ (Mwt: 213.89): 5 mg (23.3 μmol) in water (1.0 ml)
1-hexadecanoyl-4-(2-aminoxy-1-ethyl)piperidine (Mwt: 382.6): 3.6 mg in DMF (0.2 ml)
AcOH (Mwt: 60, d: 1): 17 μl (283 μmol) in water (1.0 ml)

Ser-hGH (Mwt: 22211; approx 1 mg, 45 nmol) was dissolved in the buffer (110 PI). To this solution was added the methionine solution (10 μl, approx 30 eq) followed by the NaIO₄ solution (10 μl, approx 5 eq). The clear solution was kept at room temperature for 15 min. A solution of the hydroxylamine (10 μl; approx 10 eq) was added and the resulting, slightly turbid mixture was kept at room temperature for 15 min.

Then the solution of AcOH (10 μl, approx 60 eq) was added, and after approx 1 min water (0.5 ml) and the above triethanolamine buffer (0.5 ml) were added. Analysis by HPLC and Maldi indicated at least 80% conversion to the desired oxime.

### Example 3

### 1-[2-(O-(4-(4-(Methoxy-PEG(30K)-oxy)butyrylamino)butyl)oximino)acetyl]-hGH

### (a) N-(tert-Butyloxycarbonylaminoxybutyl)phthalimide

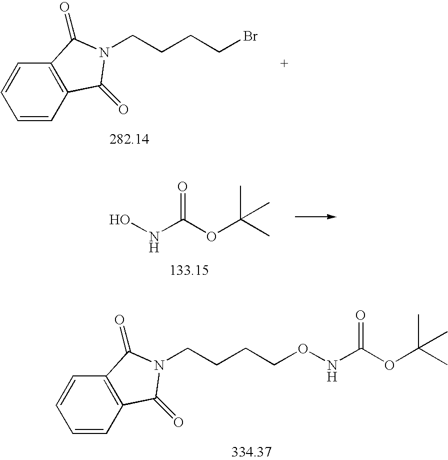

To a stirred mixture of N-(4-bromobutyl)phthalimide (18.9 g, 67.0 mmol), MeCN (14 ml), and N-Boc-hydroxylamine (12.7 g, 95.4 mmol) was added DBU (15.0 ml, 101 mmol) in portions. The resulting mixture was stirred at 50° C. for 24 h. Water (300 ml) and 12 M HCl (10 ml) were added, and the product was extracted three times with AcOEt. The combined extracts were washed with brine, dried (MgSO₄), and concentrated under reduced pressure. The resulting oil (28 g) was purified by chromatography (140 g SiO₂, gradient elution with heptane/AcOEt). 17.9 g (80%) of the title compound was obtained as an oil. ¹H NMR (DMSO-d₆) δ 1.36 (s, 9H), 1.50 (m, 2H), 1.67 (m, 2H), 3.58 (t, J=7 Hz, 2H), 3.68 (t, J=7 Hz, 2H), 7.85 (m, 4H), 9.90 (s, 1H).

### (b) 4-(tert-Butyloxycarbonylaminoxy)butylamine

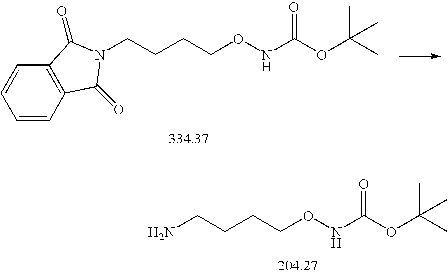

To a solution of N-(tert-butyloxycarbonylaminoxybutyl)phthalimide obtained from (a) (8.35 g, 25.0 mmol) in EtOH (10 ml) was added hydrazine hydrate (20 ml), and the mixture was stirred at 80° C. for 38 h. The mixture was concentrated and the residue coevaporated with EtOH and PhMe. To the residue was added EtOH (50 ml), and the precipitated phthalhydrazide was filtered off and washed with EtOH (50 ml). Concentration of the combined filtrates yielded 5.08 g of an oil. This oil was mixed with a solution of K₂CO₃ (10 g) in water (20 ml), and the product was extracted with CH₂Cl₂. Drying (MgSO₄) and concentration yielded 2.28 g (45%) of the title compound as an oil, which was used without further purification. ¹H NMR (DMSO-d₆) δ 1.38 (m, 2H), 1.39 (s, 9H), 1.51 (m, 2H), 2.51 (t, J=7 Hz, 2H), 3.66 (t, J=7 Hz, 2H).

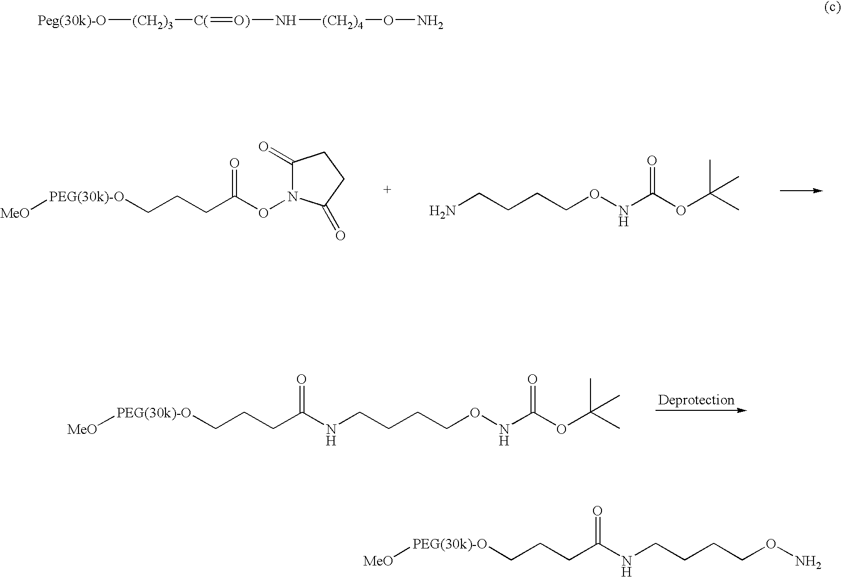

To a solution of 4-(N-butyloxycarbonyl-aminoxy)butylamine obtained in (b) (0.43 g, 2.10 mmol) in CH₂Cl₂ (40 ml) was added mPEG-SBA(30K) (Nektar Therapeutics, 5.0 g, 0.17 mmol). The resulting mixture was stirred at room temperature for 5 d. The mixture was concentrated and the residue dried under reduced pressure. Recrystallization from iPrOH (4×80 ml) yielded 4.14 g of the Boc-protected Peg(30K)-alkoxylamine.

To this product (21 mg, approx 0.7 μmol) were added CH₂Cl₂ (2.5 ml) and TFA (2.5 ml). After 0.5 h the mixture was concentrated, the residue redissolved in CH₂Cl₂ (2.5 ml) and toluene (10 ml) and concentrated again, and the residue was transferred to a smaller vial with CH₂Cl₂ (3 ml), and concentrated with a stream of N₂, then under reduced pressure. The residue was dissolved in water (0.21 ml) and with 2-methylpyridine (≈5 μl) the pH was adjusted to 6.0.

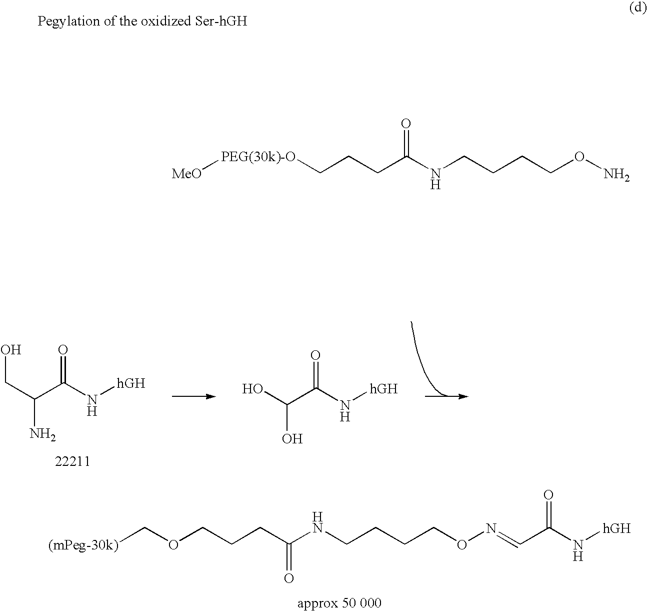

The following solutions were prepared:

Buffer: 240 mg (1.61 mmol) triethanolamine (Mwt: 149.21) in water (100 ml); pH=8.5.
Methionine (Mwt: 149.21): 26 mg (0.174 mmol) in water (1.0 ml)
NaIO₄ (Mwt: 213.89): 5 mg (24 μmol) in water (1.0 ml)

To Ser-hGH (1 mg, 45 nmol) was added the buffer (0.10 ml) and the methionine solution (0.030 ml), and the periodate solution (15 μl) was added. After 15 min DMF (60 μl) was added, and the whole mixture was added to the Peg-solution obtained in (c). The resulting mixture has pH 6.5. The mixture was allowed to stand for 20 h, and was then analyzed by SDS-PAGE. The pegylated hGH was obtained in approximately 30% yield.

### Example 4

### 2,3-Bis(methylpolyoxyethyleneoxy)-1-({3-[(1,5-dioxo-5-(4-boc-aminoxybutylamino)pentyl)amino]propyl}polyoxyethyleneoxy)propane

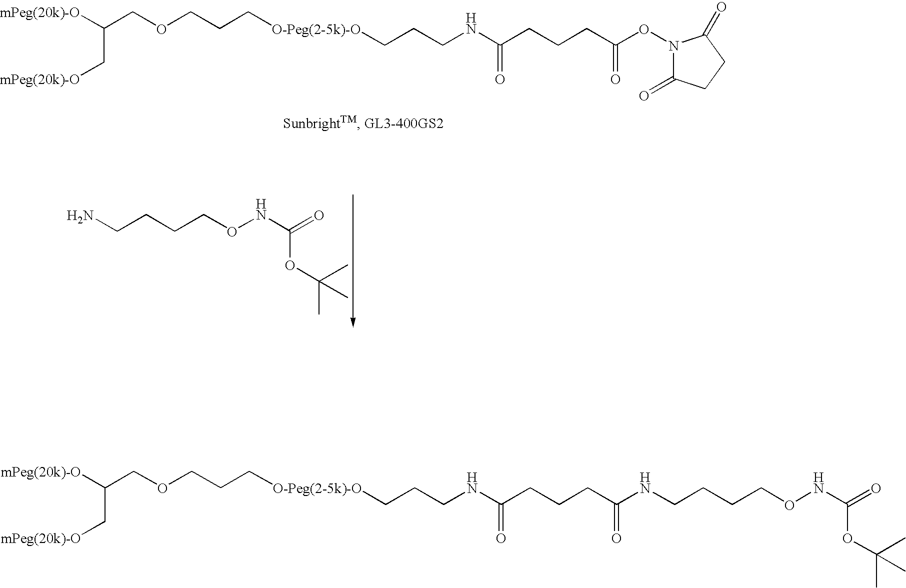

To a solution of 4-(N-Boc-aminoxy)-1-butylamine (0.3 g, 1.5 mmol) in DCM (20 ml) was added the Peg-OSu ester (5 g, 0.125 mmol; Sunbright GL3-400GS2 supplied by NOF Corp.), followed by the addition of DIPEA (0.5 ml). The mixture was stirred at room temperature for five days. The product was precipitated by addition of diethylether, isolated by filtration, redissolved in DCM and precipitated three more times. The product was then dissolved in a mixture of DCM and MeOH, and acidic ion exchange resin (Amberlyst 15, 10 g, washed with DCM+MeOH) was added. The mixture was stirred for 1 h, filtered and concentrated, and the residue purified by three more precipitations with diethylether. The amount of Boc-groups was determined by 1H NMR spectroscopy with an internal standard (4-cyano-4′-hydroxybiphenyl) to be 12.8 μmol/g.

### Example 5

### Pegylation of oxidized SerhGH: 1-(N-{4-(5-[3-(omega-(3-(2,3-Bis(methylpolyoxyethyleneoxy)propyloxy)propyl)polyoxyethyleneoxy)propylamino]-1,5-dioxopentylamino)butoxy}iminoacetyl)hGH

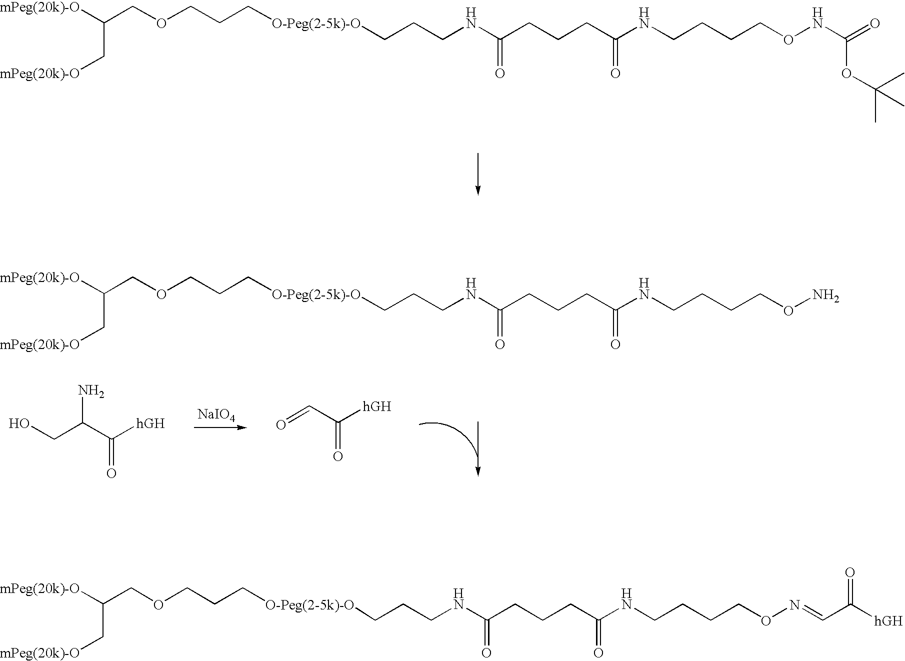

The following buffers were used:

Buffer A: triethanolamine (270 mg, 1.8 mmol), 3-methylthiopropanol (580 mg, 5.46 mmol), water (40 ml).
Buffer E: MES-hydrate (4.27 g, 20 mmol)+water (50 ml)+1 N NaOH (12.5 ml, 12.5 mmol). pH=6.5.
Buffer F: L-methionine (2.0 g)+water (80 ml).
Periodate solution: NaIO₄ (48.1 mg, 0.225 mmol)+water (1.0 ml).

### Peg-Deprotection:

The product prepared in Example 2 (200 mg, 2.5 μmol) was mixed with DCM (15 ml) and TFA (15 ml), and after 0.5 h the mixture was concentrated, and the residue coevaporated once with EtOH and dried overnight under reduced pressure. The residue was dissolved in buffer E (4.0 ml).

### Oxidation:

To a solution of SerhGH (60 mg, 2.7 μmol) in buffer A (6 ml) the periodate solution (0.6 ml) was added. After 15 min the mixture was diluted with buffer F and dialyzed five times with buffer F (Amicon Ultra, cut-off 5000 Da, 10° C.). The residue was diluted with buffer F to 1.5 ml and to this solution ice-cold (0° C.) NMP (0.5 ml) was added.

### Oximation:

The resulting aldehyde-solution was added at once to the Peg-solution. The slightly turbid mixture was kept at room temperature in the dark for one week. The pegylated protein was purified by ion-exchange chromatography.

### Pharmacological Methods

### Assay (I) BAF-3GHR Assay to Determine Growth Hormone Activity

The BAF-3 cells (a murine pro-B lymphoid cell line derived from the bone marrow) was originally IL-3 dependent for growth and survival. Il-3 activates JAK-2 and STAT which are the same mediators GH is activating upon stimulation. After transfection of the human growth hormone receptor the cell line was turn into a growth hormone-dependent cell line. This clone can be used to evaluate the effect of different growth hormone samples on the survival of the BAF-3 GHR.

The BAF-3 GHR cells are grown in starvation medium (culture medium without growth hormone) for 24 hours at 37° C., 5% CO₂.

The cells are washed and re-suspended in starvation medium and seeded in plates. 10 μl of growth hormone compound or human growth hormone in different concentrations or control is added to the cells, and the plates are incubated for 68 hours at 37° C., 5% CO₂.

AlamarBlue® is added to each well and the cells are then incubated for another 4 hours. The AlamarBlue® is a redox indicator, and is reduced by reactions innate to cellular metabolism and, therefore, provides an indirect measure of viable cell number.

Finally, the metabolic activity of the cells is measure in a fluorescence plate reader. The absorbance in the samples is expressed in % of cells not stimulated with growth hormone compound or control and from the concentration-response curves the activity (amount of a compound that stimulates the cells with 50%) can be calculated.

1. A compound according to formula (I) 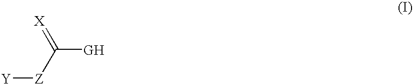
wherein GH represent a radical derived from a growth hormone compound by removal of one hydrogen atom from the N-terminal amino group;
X represents oxygen or two hydrogen atoms;
Z represents a bond, alkylene, arylene, heteroarylene, —CH₂—O—(CH₂)₁₋₁₀—, —CH₂—O—(C₆H₄)—, or any combination thereof;
Y represents a radical selected from 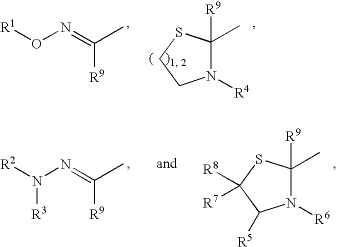
wherein
R¹ and R⁴ independently represent MeO—(CH₂CH₂O)₁₀₀₀₋₁₀₀₀-E-, [MeO—(CH₂CH₂O)₁₀₀₋₁₀₀₀]—C(═O)NH—(CH₂)₄CH[MeO—(CH₂CH₂O)₁₀₀₋₁₀₀₀—C(═O)NH]E-, Me-(CH₂)₁₀₋₃₀-E-, Me-(CH₂)₁₀₋₃₀—C(═O)—NH—S(═O)₂—(CH₂)₂₋₁₀-E-, (5-tetrazolyl)-(CH₂)₁₋₃₀-E-, (5-tetrazolyl)₂CH—(CH₂)₁₋₃₀-E-, (5-tetrazolyl)-(CH₂)₁₋₃₀—C(═O)—NH—S(═O)₂—(CH₂)₂₋₁₀-E-, (5-tetrazoly)-(C₆H₄)—O—(CH₂) 1-30, (5-tetrazoly)-(C₆H₄)—(C₆H₄)—O—(CH₂)₁₋₃₀—, HO₂C—(CH₂)₁₀₋₃₀-E-, cibacronyl-E-, GH-C(═O)—CH═N—O—(CH₂)₂₋₃₀—, GH-C(═O)—CH═N—(OCH₂CH₂)₁₋₁₀—, GH-(C═O)—CH═N—NH—(CH₂)₂₋₃₀—, GH-(C═O)—CH═N—NH—(CH₂CH₂O)₁₋₃₀—(CH₂)₀₋₃₀—, 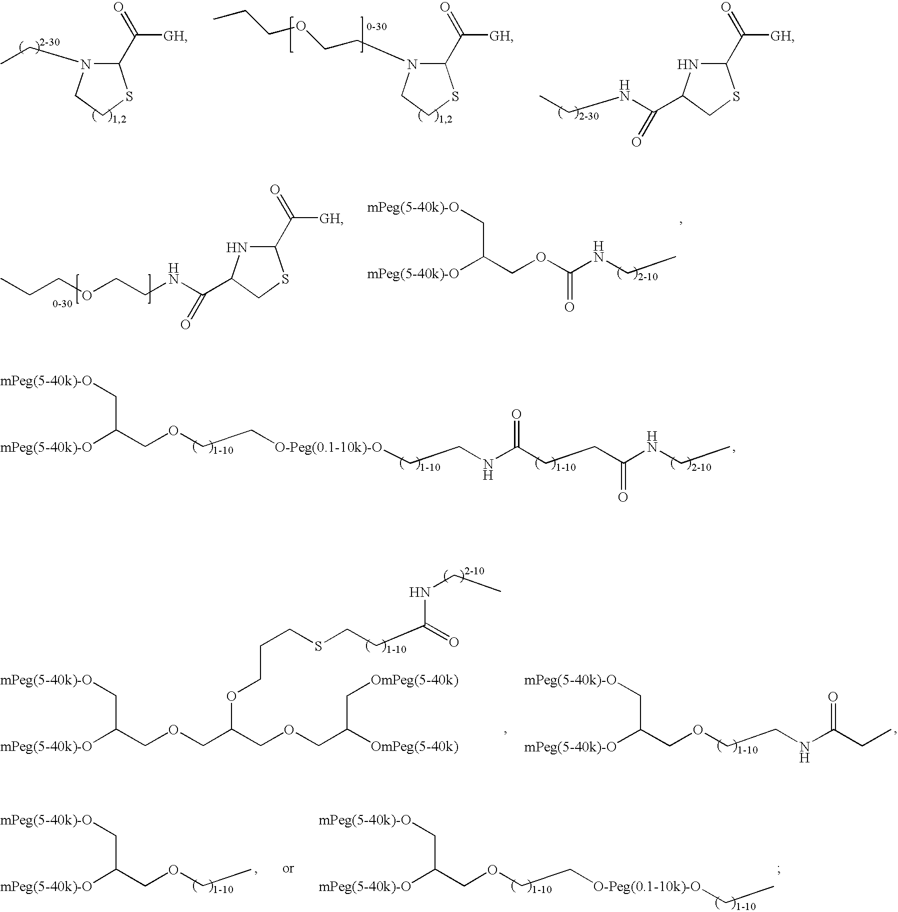
R², R³, R⁵ and R⁶ independently represent
H, C₁₋₆alkyl, MeO—(CH₂CH₂O)₁₋₁₀₀₀-E-, [MeO—(CH₂CH₂O)₁₋₁₀₀₀]—C(═O)NH—(CH₂)₄CH[MeO—(CH₂CH₂O)₁₋₁₀₀₀—C(═O)NH]E-, Me-(CH₂)₁₋₃₀-E-, Me-(CH₂)₁₀₋₃₀—C(═O)—NH—S(═O)₂—(CH₂)₂₋₁₀-E-, (5-tetrazolyl)-(CH₂)₁₀₋₃₀-E-, (5-tetrazolyl)₂CH—(CH₂)₁₀₋₃₀-E-, (5-tetrazolyl)-(CH₂)₁₋₃₀—C(═O)—NH—S(═O)₂—(CH₂)₂₋₁₀-E-, HO₂C—(CH₂)₁₀₋₃₀-E-, cibacronyl-E-, GH-C(═O)—CH═N—O—(CH₂)₂₋₃₀—, GH-C(═O)—CH═N—O—(OCH₂CH₂)₁₋₁₀—, GH-(C═O)—CH═N—NH—(CH₂)₂₋₃₀—, GH-(C═O)—CH—═N—NH—(CH₂CH₂O)₁₋₃₀—(CH₂)₀₋₃₀—, 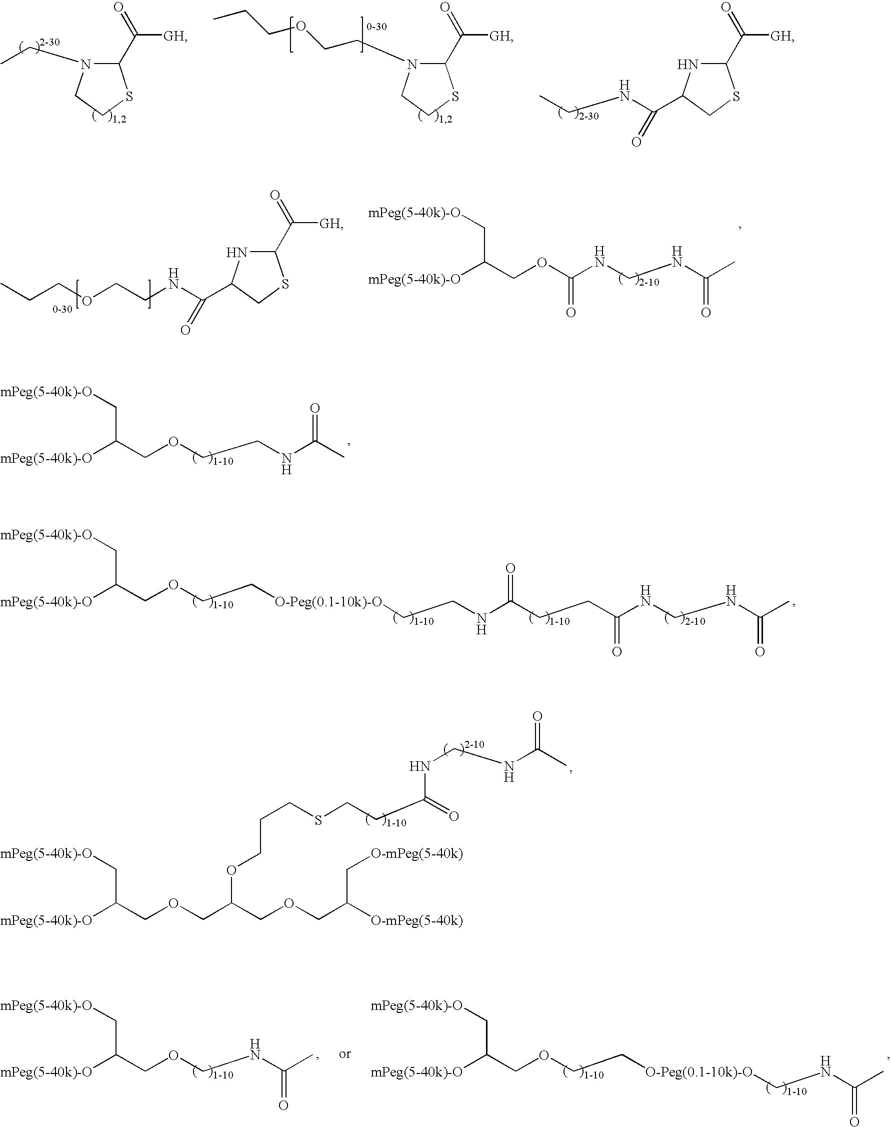
provided that at least one of R² and R³, or at least one of R⁵ and R⁶, does not represent H or C₁₋₆alkyl;
E represents a bond or a diradical selected from
—C(═O)NH(CH₂)₂₋₃₀—, —(CH₂)₁₋₃₀C(═O)NH(CH₂)₂₋₃₀—, —(CH₂)₀₋₃₀C(═O)NH—(CH₂CH₂O)₁₋₁₀—(CH₂)₁₋₅—C(═O)—, —C(═O)NH—[(CH₂CH₂O)₁₋₁₀—(CH₂)₁₋₅—C(═O)]₁₋₅NH(CH₂)₂₋₃₀—, —C(═O)—, —NHC(═O)—, —C(═O)NH—, —(CH₂)₁₋₃₀NHC(═O)—, —(CH₂)₁₋₃₀C(═O)—, —NHC(═O)NH(CH₂)₂₋₃₀—, —(CH₂)₁₋₃₀NHC(═O)NH(CH₂)₂₋₃₀—, 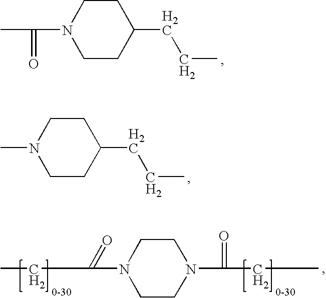
—(CH₂)₀₋₃₀C(═O)NH(CH₂)₂₋₃₀—NHC(═O)—(CH₂)₀₋₃₀—, —(CH₂)₀₋₃₀C(═O)NH(CH₂CH₂O)₁₋₃₀—CH₂CH₂NHC(═O)—(CH₂)₀₋₃₀—, —NH(CH₂)₂₋₃₀—, —NH—, —O—, —S—; and
R⁷, R⁸ and R⁹ independently represent H, C₁₋₆alkyl, aryl, or heteroaryl;
and pharmaceutically acceptable salts, prodrugs and solvates thereof.

2. The compound according to claim 1, wherein X represents two hydrogens.

3. The compound according to claim 1, wherein Y represents

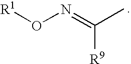

4. The compound according to claim 3, wherein R⁹ represents hydrogen.

5. The compound according to claim 1, wherein, R¹ represents MeO—(CH₂CH₂O)₁₀₀₋₁₀₀₀-E- or [MeO—(CH₂CH₂O)₁₀₀₋₁₀₀₀]—C(═O)NH—(CH₂)₄CH[MeO—(CH₂CH₂O)₁₀₀₋₁₀₀₀—C(═O)NH]E-.

6. The compound according to claim 5, wherein, R¹ represents MeO—(CH₂CH₂O)₄₀₀₋₁₀₀₀-E- or [MeO—(CH₂CH₂O)₃₀₀₋₆₀₀]—C(═O)NH—(CH₂)₄CH[MeO—(CH₂CH₂O)₃₀₀₋₆₀₀—C(═O)NH]E-.

7. The compound according to claim 5, wherein R¹ represents MeO—(CH₂CH₂O)₄₀₀₋₅₀₀-E-, MeO—(CH₂CH₂O)₆₀₀₋₇₀₀-E- or MeO—(CH₂CH₂O)₈₅₀₋₉₅₀-E-; or [MeO—(CH₂CH₂O)₄₀₀₋₅₀₀]—C(═O)NH—(CH₂)₄CH[MeO—(CH₂CH₂O)₄₀₀₋₅₀₀—C(═O)NH]E-.

8. The compound according to claim 7, wherein E represents —C(═O)NH(CH₂)₂₋₃₀— or —(CH₂)₁₋₃₀C(═O)NH(CH₂)₂₋₃₀—.

9. The compound according to claim 8, wherein E represents —C(═O)NH(CH₂)₂₋₁₀ or —(CH₂)₁₋₁₀C(═O)NH(CH₂)₂₋₁₀—.

10. The compound according to claim 8, wherein E represents —C(═O)NH(CH₂)₄ or —(CH₂)₃C(═O)NH(CH₂)₄—.

11. The compound according to claim 2, wherein R¹ is selected is selected from
MeO—(CH₂CH₂O)₄₀₀₋₅₀₀—CH₂CH₂CH₂—C(═O)—NH—(CH₂)₄—,
MeO—(CH₂CH₂O)₆₀₀₋₇₀₀—CH₂CH₂CH₂—C(═O)—NH—(CH₂)₄—,
MeO—(CH₂CH₂O)₈₅₀₋₉₅₀—CH₂CH₂CH₂—C(═O)—NH—(CH₂)₄—, and
MeO—(CH₂CH₂O)₄₀₀₋₅₀₀—C(═O)NH—(CH₂)₄CH[MeO—(CH₂CH₂O)₄₀₀₋₅₀₀—C(═O)NH]—,

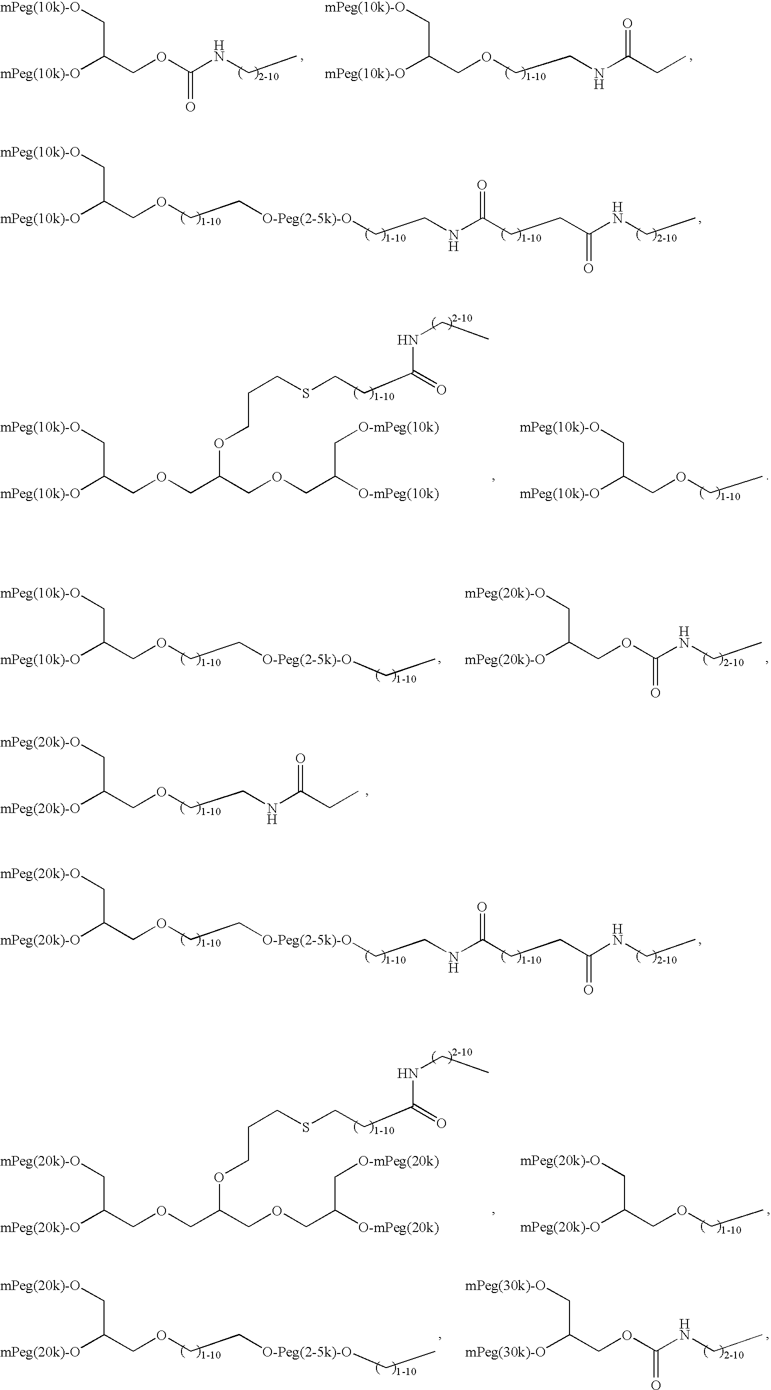

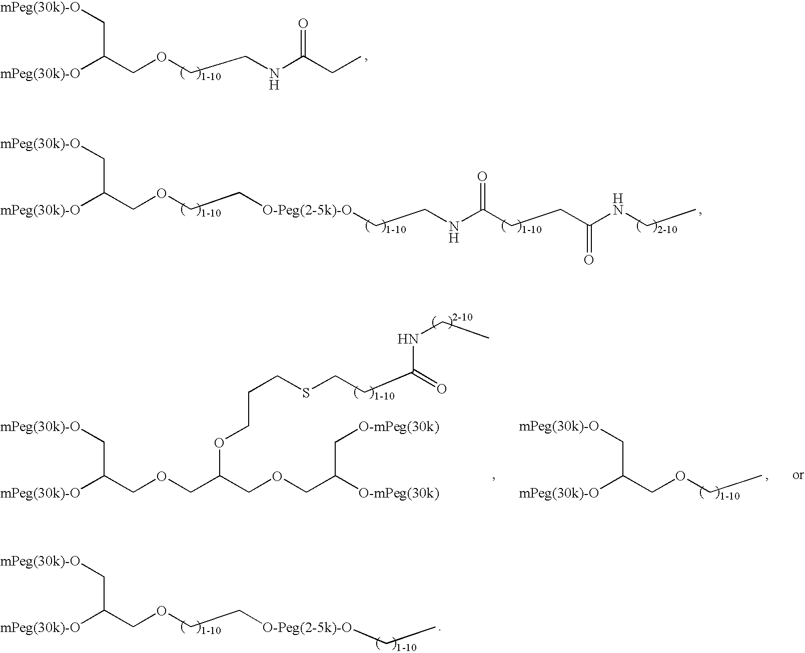

12. The compound according to claim 2, wherein Z represents —CH₂—O—(CH₂)₁₋₅ or —CH—O—(C₆H₄)—.

13. The compound according to claim 12, wherein Z represents —CH₂—O—(CH₂)₃, —CH₂—O—(CH₂)₄, —CH₂—O—(CH₂)₅, —CH₂—O-(1,4-C₆H₄)—, or —CH₂—O-(1,3-C₆H₄)—.

14. The compound according to claim 1, wherein X represents O and Z represents a bond.

15. The compound according to claim 14, wherein Y represents

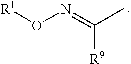

16. The compound according to claim 14, wherein R⁹ represents hydrogen.

17. The compound according to claim 14, wherein R¹ represents

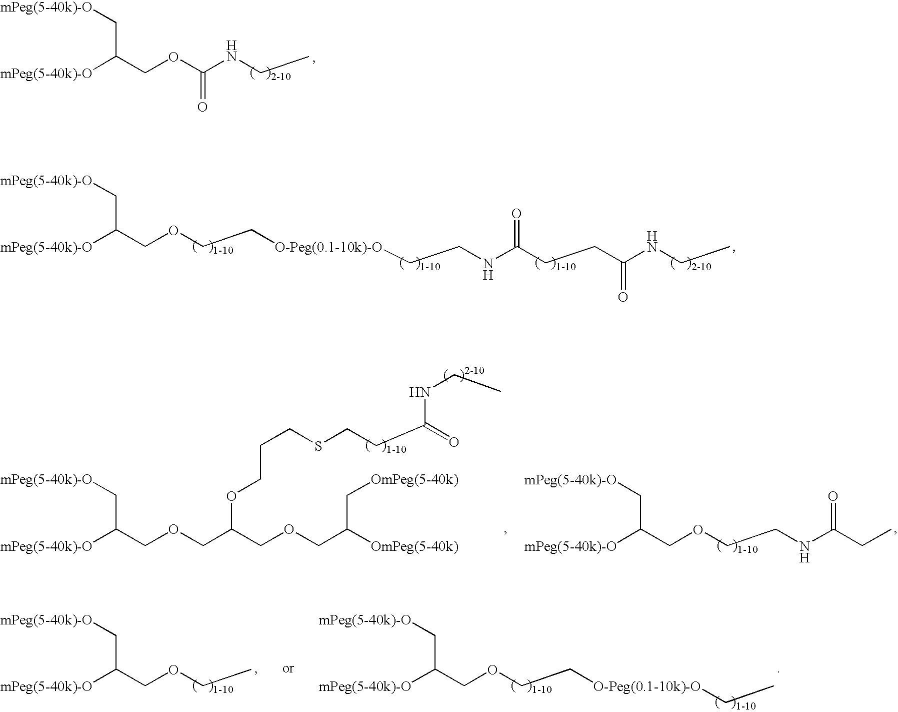

18. The compound according to claim 14, wherein R¹ represents

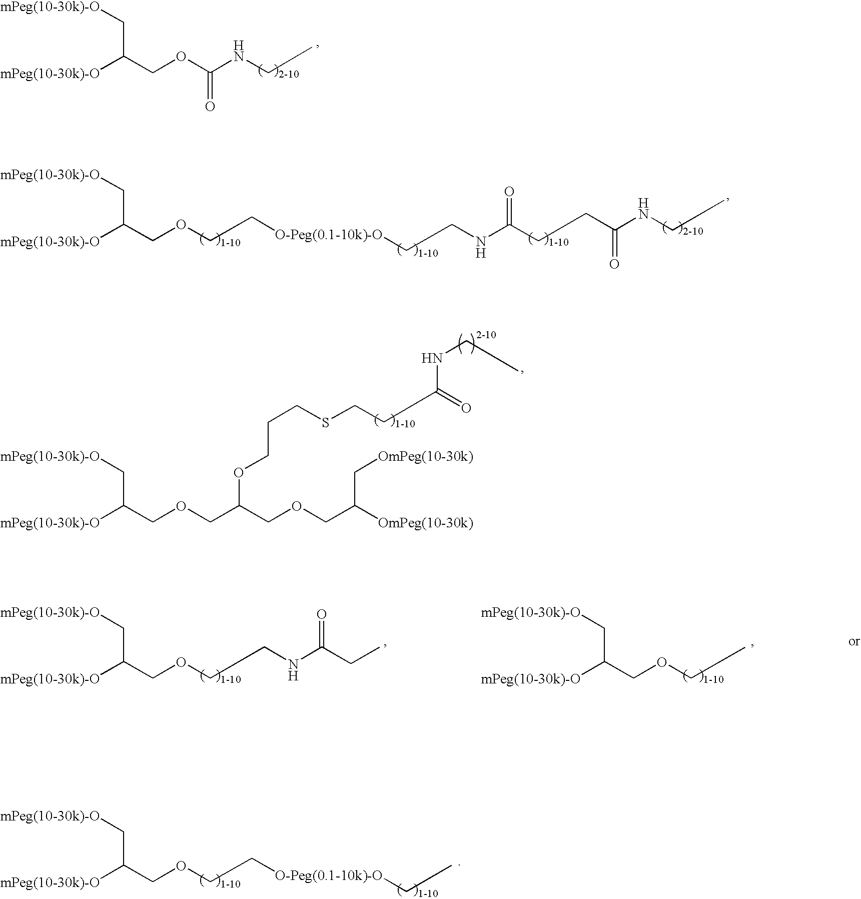

19. The compound according to claim 14, wherein R¹ represents MeO—(CH₂CH₂O)₁₀₀₋₁₀₀₀-E-, [MeO—(CH₂CH₂O)₁₀₀₋₁₀₀₀]—C(═O)NH—(CH₂)₄CH[MeO—(CH₂CH₂O)₁₀₀₋₁₀₀₀—C(═O)NH]E-, Me-(CH₂)₁₀₋₃₀-E-, (5-tetrazolyl)-(CH₂)₁₋₃₀-E-, (5-tetrazolyl)₂CH—(CH₂)₁₋₃₀-E-, (5-tetrazolyl)-(CH₂)₁₋₃₀—C(═O)—NH—S(═O)₂—(CH₂)₁₋₁₀, (5-tetrazoly)-(C₆H₄)—O—(CH₂)₁₋₃₀, (5-tetrazoly)-(C₆H₄)—(C₆H₄)—O—(CH₂)₁₋₃₀, GH-C(═O)—CH═N—(OCH₂CH₂)₁₋₁₀—, or cibacronyl-E-.

20. The compound according to claim 19, wherein R¹ represents MeO—(CH₂CH₂O)₄₀₀₋₁₀₀₀-E-, [MeO—(CH₂CH₂O)₄₀₀₋₈₀₀]—C(═O)NH—(CH₂)₄CH[MeO—(CH₂CH₂O)₄₀₀₋₈₀₀—C(═O)NH]E-, Me-(CH₂)₁₀₋₂₀-E-, (5-tetrazolyl)-(CH₂)₁₋₂₀-E-, (5-tetrazolyl)₂CH—(CH₂)₁₋₂₀-E-, (5-tetrazolyl)-(CH₂)₁₋₂₀—C(═O)—NH—S(═O)₂—(CH₂)₁—, (5-tetrazoly)-(C₆H₄)—O—(CH₂)₁₋₂₀, (5-tetrazoly)-(C₆H₄)—(C₆H₄)—O—(CH₂)₁₋₂₀—, GH-C(═O)—CH═N—(OCH₂CH₂)₁₋₈—, or cibacronyl-E-.

21. The compound according to claim 20, wherein R¹ represents MeO—(CH₂CH₂O)₄₀₀₋₅₀₀-E-, MeO—(CH₂CH₂O)₆₀₀₋₇₀₀-E-, MeO—(CH₂CH₂O)₈₅₀₋₉₅₀-E-, [MeO—(CH₂CH₂O)₄₀₀₋₅₀₀]—C(═O)NH—(CH₂)₄CH[MeO—(CH₂CH₂O)₄₀₀₋₅₀₀—C(═O)NH]E-, [MeO—(CH₂CH₂O)₆₀₀₋₇₀₀]—C(═O)NH—(CH₂)₄CH[MeO—(CH₂CH₂O)₆₀₀₋₇₀₀—C(═O)NH]E-, Me-(CH₂)₁₂-E-, Me-(CH₂)₁₀₋₂₀-E-, Me-(CH₂)₁₄-E-, Me-(CH₂)₁₆-E-, Me-(CH₂)₁₈-E-, (5-tetrazolyl)-(CH₂)₁₅-E-, (5-tetrazolyl)₂CH—(CH₂)₁₄-E-, (5-tetrazolyl)-(CH₂)₁₅—C(═O)—NH—S(═O)₂—(CH₂)₄, (5-tetrazoly)-(C₆H₄)—O—(CH₂)₁₅, (5-tetrazoly)-(C₆H₄)—(C₆H₄)—O—(CH₂)₁₋₅—, or cibacronyl-E-.

22. The compound according to claim 14, wherein E represents —C(═O)NH(CH₂)₂₋₃₀—, —(CH₂)₁₋₃₀C(═O)NH(CH₂)₂₋₃₀—, 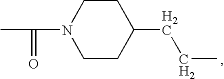
—C(═O)NH—[(CH₂CH₂O)₁₋₁₀—(CH₂)₁₋₅—C(═O)]₁₋₅NH(CH₂)₂₋₃₀—, or a bond.

23. The compound according to claim 22, wherein, E represents —C(═O)NH(CH₂)₂₋₁₀—, —(CH₂)₁₋₁₀C(═O)NH(CH₂)₂₋₁₀—, 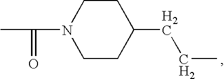
—C(═O)NH—[(CH₂CH₂O)₁₋₅—(CH₂)₁—C(═O)]₁₋₄NH(CH₂)₂₋₁₀—, or a bond.

24. The compound according to claim 22, wherein, E represents —C(═O)NH(CH₂)₄—, —(CH₂)₃C(═O)NH(CH₂)₄, 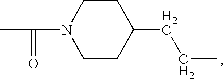
—C(═O)NH—[(CH₂CH₂O)₂—(CH₂)₁—C(═O)]₁NH(CH₂)₄—, —C(═O)NH-[(CH₂CH₂O)₂—(CH₂)₁—C(═O)]₂NH(CH₂)₄—, —C(═O)NH—[(CH₂CH₂O)₂—(CH₂)₁—C(═O)]₃NH(CH₂)₄—, and a bond.

25. The compound according to claim 14, wherein R¹ represents
MeO—(CH₂CH₂O)₄₀₀₋₅₀₀—CH₂CH₂CH₂—C(═O)—NH—(CH₂)₄—,
MeO—(CH₂CH₂O)₆₀₀₋₇₀₀—CH₂CH₂CH₂—C(═O)—NH—(CH₂)₄—,
MeO—(CH₂CH₂O)₈₅₀₋₉₅₀—CH₂CH₂CH₂—C(═O)—NH—(CH₂)₄—,
MeO—(CH₂CH₂O)₄₀₀₋₅₀₀—C(═O)NH—(CH₂)₄CH[MeO—(CH₂CH₂O)₄₀₀₋₅₀₀—C(═O)NH]—C(═O)NH—(CH₂)₄—,
MeO—(CH₂CH₂O)₆₀₀₋₇₀₀—C(═O)NH—(CH₂)₄CH[MeO—(CH₂CH₂O)₆₀₀₋₇₀₀—C(═O)NH]—C(═O)NH—(CH₂)₄— 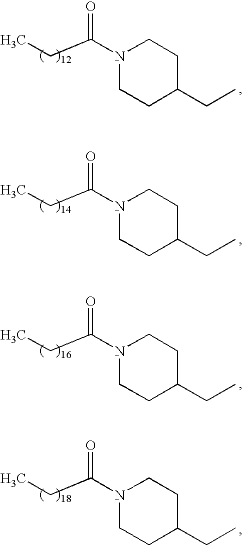
(5-tetrazolyl)-(CH₂)₁₅—C(═O)—NH—(CH₂)₄—,
(5-tetrazolyl)-(CH₂)₁₋₅—C(═O)—NH—S(═O)₂—(CH₂)₃—C(═O)—NH—(CH₂)₄—,
(5-tetrazolyl)-(CH₂)₁₅—C(═O)—NH—(CH₂CH₂—O)₂—CH₂—C(═O)NH—(CH₂)₄—,
(5-tetrazolyl)-(CH₂)₁₋₅—C(═O)-[NH—(CH₂CH₂—O)₂—CH₂—C(═O)]₂NH—(CH₂)₄—,
(5-tetrazolyl)-(CH₂)₁₅—C(═O)-[NH—(CH₂CH₂—O)₂—CH₂—C(═O)]₃NH—(CH₂)₄—,
(5-tetrazolyl)₂CH—(CH₂)₁₋₄—C(═O)—NH—(CH₂)₄—
(5-tetrazolyl)(1,4-C₆H₄)—O—(CH₂)₁₅—C(═O)—NH—(CH₂)₄—
(5-tetrazolyl)(1,4-C₆H₄)₂—O—(CH₂)₁₅—C(═O)—NH—(CH₂)₄—
GH-C(═O)—CH₂—CH═N—(O—CH₂CH₂)₂—,
GH-C(═O)—CH₂—CH═N—(O—CH₂CH₂)₃—,
GH-C(═O)—CH₂—CH═N—(O—CH₂CH₂)₄—,
GH-C(═O)—CH₂—CH═N—(O—CH₂CH₂)₅—,
GH-C(═O)—CH₂—CH═N—(O—CH₂CH₂)₆—,
cibacronyl-NH—(CH₂)₄—,

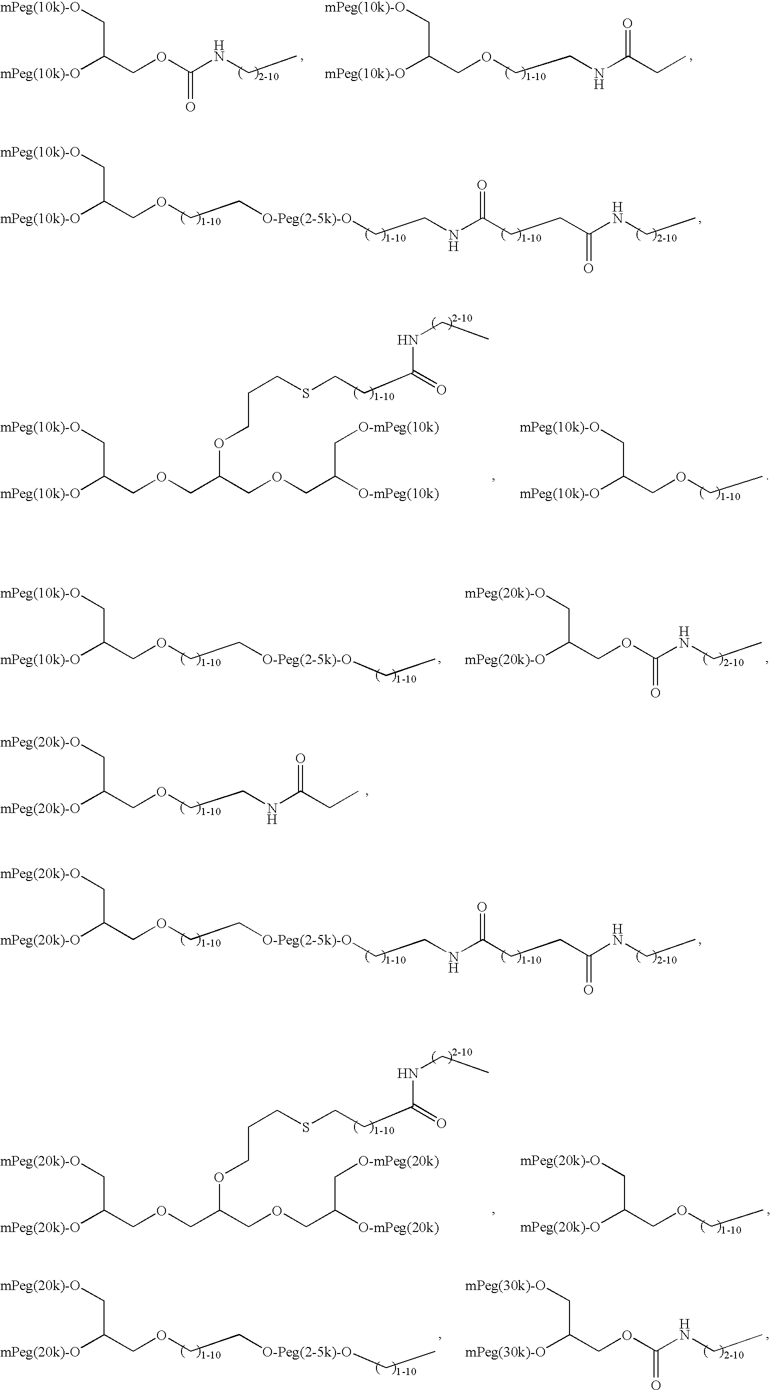

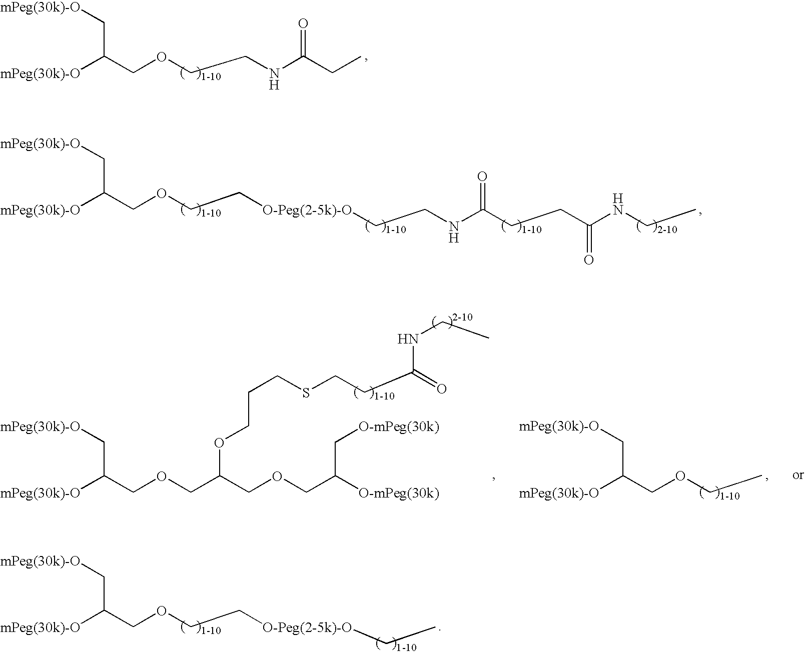

26. The compound according to claim 1 selected from
[MeO—(CH₂CH₂O)₄₀₀₋₅₀₀—CH₂CH₂—NH—C(═O)—O—CH₂]₂CH—O—(CH₂)₃—C(═O)—NH—(CH₂) N═CH—C(═O)-GH,
[MeO—(CH₂CH₂O)₆₀₀₋₇₅₀](CH₂)₃—C(═O)NH—(CH₂)₄CH[MeO—(CH₂CH₂O)₆₀₀₋₇₅₀—(CH₂)₃C(═O)NH]—C(═O)NH—(CH₂)₄—O—N═CH—C(═O)-GH,
MeO—(CH₂CH₂O)₄₀₀₋₅₀₀—CH₂CH₂CH₂—C(═O)—NH—(CH₂)₄—O—N═CH—C(═O)-GH,
MeO—(CH₂CH₂O)₆₀₀₋₇₀₀—CH₂CH₂CH₂—C(═O)—NH—(CH₂)₄—O—N═CH—C(═O)-GH,
MeO—(CH₂CH₂O)₈₅₀₋₉₅₀—CH₂CH₂CH₂—C(═O)—NH—(CH₂)₄—O—N═CH—C(═O)-GH,
[MeO—(CH₂CH₂O)₄₀₀₋₅₀₀]—C(═O)NH—(CH₂)₄CH[MeO—(CH₂CH₂O)₄₀₀₋₅₀₀—C(═O)NH]—C(═O)NH—
(CH₂)₄—O—N═CH—C(═O)-GH,
[MeO—(CH₂CH₂O)₆₀₀₋₇₀₀]—C(═O)NH—(CH₂)₄CH[MeO—(CH₂CH₂O)₆₀₀₋₇₀₀—C(═O)NH]—C(═O)NH—(CH₂)₄—O—N═CH—C(═O)-GH, 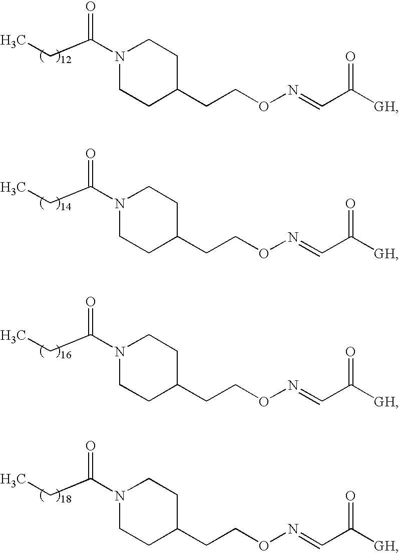
(5-tetrazolyl)-(CH₂)₁₅—C(═O)—NH—(CH₂)₄—O—N═CH—C(═O)-GH,
(5-tetrazolyl)-(CH₂)₁₋₅—C(═O)—NH—S(═O)₂—(CH₂)₃—C(═O)—NH—(CH₂)₄—O—N═CH—C(═O)-GH,
(5-tetrazolyl)-(CH₂)₁₅—C(═O)—NH—(CH₂CH₂—O)₂—CH₂—C(═O)NH—(CH₂)₄—O—N═CH—C(═O)-GH,
(5-tetrazolyl)-(CH₂)₁₋₅—C(═O)—[NH—(CH₂CH₂—O)₂—CH₂—C(═O)]₂NH—(CH₂)₄—O—N═CH—C(═O)-GH,
(5-tetrazolyl)-(CH₂)₁₋₅—C(═O)—[NH—(CH₂CH₂—O)₂—CH₂—C(═O)]₃NH—(CH₂)₄—O—N═CH—C(═O)-GH,
(5-tetrazolyl)₂CH—(CH₂)₁₋₄—C(═O)—NH—(CH₂)₄—O—N═CH—C(═O)-GH,
(5-tetrazolyl)(1,4-C₆H₄)—O—(CH₂)₁₅—C(═O)—NH—(CH₂)₄—O—N═CH—C(═O)-GH,
(5-tetrazolyl)(1,4-C₆H₄)₂—O—(CH₂)₁₅—C(═O)—NH—(CH₂)₄—O—N═CH—C(═O)-GH,
GH-C(═O)—CH═N—(O—CH₂CH₂)₂—O—N═CH—C(═O)-GH,
GH-C(═O)—CH═N—(O—CH₂CH₂)₃—O—N═CH—C(═O)-GH,
GH-C(═O)—CH═N—(O—CH₂CH₂)₄—O—N═CH—C(═O)-GH,
GH-C(═O)—CH═N—(O—CH₂CH₂)₅—O—N═CH—C(═O)-GH,
GH-C(═O)—CH═N—(O—CH₂CH₂)₆—O—N═CH—C(═O)-GH,
cibacronyl-NH—(CH₂)₄—O—N═CH—C(═O)-GH,
MeO—(CH₂CH₂O)₄₀₀₋₅₀₀—CH₂CH₂CH₂—C(═O)—NH—(CH₂)₄—O—N═CH—CH₂—O—(CH₂)₄-GH,
MeO—(CH₂CH₂O)₆₀₀₋₇₀₀—CH₂CH₂CH₂—C(═O)—NH—(CH₂)₄—O—N═CH—CH₂—O—(CH₂)₄-GH,
MeO—(CH₂CH₂O)₈₅₀₋₉₅₀—CH₂CH₂CH₂—C(═O)—NH—(CH₂)₄—O—N═CH—CH₂—O—(CH₂)₄-GH,
[MeO—(CH₂CH₂O)₄₀₀₋₅₀₀]—C(═O)NH—(CH₂)₄CH[MeO—(CH₂CH₂O)₄₀₀₋₅₀₀—C(═O)NH]—C(═O)NH—(CH₂)₄—O—N═CH—CH₂—O—(CH₂)₄-GH,
MeO—(CH₂CH₂O)₄₀₀₋₅₀₀—CH₂CH₂CH₂—C(═O)—NH—(CH₂)₄—O—N═CH—CH₂—O—(CH₂)₅-GH,
MeO—(CH₂CH₂O)₆₀₀₋₇₀₀—CH₂CH₂CH₂—C(═O)—NH—(CH₂)₄—O—N═CH—CH₂—O—(CH₂)₅-GH,
MeO—(CH₂CH₂O)₈₅₀₋₉₅₀—CH₂CH₂CH₂—C(═O)—NH—(CH₂)₄—O—N═CH—CH₂—O—(CH₂)₅-GH,
[MeO—(CH₂CH₂O)₄₀₀₋₅₀₀]—C(═O)NH—(CH₂)₄CH[MeO—(CH₂CH₂O)₄₀₀₋₅₀₀—C(═O)NH]—C(═O)NH—(CH₂)₄—O—N═CH—CH₂—O—(CH₂)₅-GH,
MeO—(CH₂CH₂O)₄₀₀₋₅₀₀—CH₂CH₂CH₂—C(═O)—NH—(CH₂)₄—O—N═CH—CH₂—O—(CH₂)₆-GH,
MeO—(CH₂CH₂O)₆₀₀₋₇₀₀—CH₂CH₂CH₂—C(═O)—NH—(CH₂)₄—O—N═CH—CH₂—O—(CH₂)₆-GH,
MeO—(CH₂CH₂O)₈₅₀₋₉₅₀—CH₂CH₂CH₂—C(═O)—NH—(CH₂)₄—O—N═CH—CH₂—O—(CH₂)₆-GH,
[MeO—(CH₂CH₂O)₄₀₀₋₅₀₀]—C(═O)NH—(CH₂)₄CH[MeO—(CH₂CH₂O)₄₀₀₋₅₀₀—C(═O)NH]—C(═O)NH—(CH₂)₄—O—N═CH—CH₂—O—(CH₂)₆-GH,
MeO—(CH₂CH₂O)₄₀₀₋₅₀₀—CH₂CH₂CH₂—C(═O)—NH—(CH₂)₄—O—N═CH—CH₂—O-(1,4-C₆H₄)CH₂-GH,
MeO—(CH₂CH₂O)₆₀₀₋₇₀₀—CH₂CH₂CH₂—C(═O)—NH—(CH₂)₄—O—N═CH—CH₂—O-(1,4-C₆H₄)CH₂-GH,
MeO—(CH₂CH₂O)₈₅₀₋₉₅₀—CH₂CH₂CH₂—C(═O)—NH—(CH₂)₄—O—N═CH—CH₂—O-(1,4-C₆H₄)CH₂-GH,
[MeO—(CH₂CH₂O)₄₀₀₋₅₀₀]—C(═O)NH—(CH₂)₄CH[MeO—(CH₂CH₂O)₄₀₀₋₅₀₀—C(═O)NH]—C(═O)NH—(CH₂)₄—O—N═CH—CH₂—O-(1,4-C₆H₄)CH₂-GH,
MeO—(CH₂CH₂O)₄₀₀₋₅₀₀—CH₂CH₂CH₂—C(═O)—NH—(CH₂)₄—O—N═CH—CH₂—O-(1,3-C₆H₄)CH₂-GH,
MeO—(CH₂CH₂O)₆₀₀₋₇₀₀—CH₂CH₂CH₂—C(═O)—NH—(CH₂)₄—O—N═CH—CH₂—O-(1,3-C₆H₄)CH₂-GH,
MeO—(CH₂CH₂O)₈₅₀₋₉₅₀—CH₂CH₂CH₂—C(═O)—NH—(CH₂)₄—O—N═CH—CH₂—O-(1,3-C₆H₄)CH₂-GH,
[MeO—(CH₂CH₂O)₄₀₀₋₅₀₀]—C(═O)NH—(CH₂)₄CH[MeO—(CH₂CH₂O)₄₀₀₋₅₀₀—C(═O)NH]—C(═O)NH—(CH₂)₄—O—N═CH—CH₂—O-(1,3-C₆H₄)CH₂-GH,

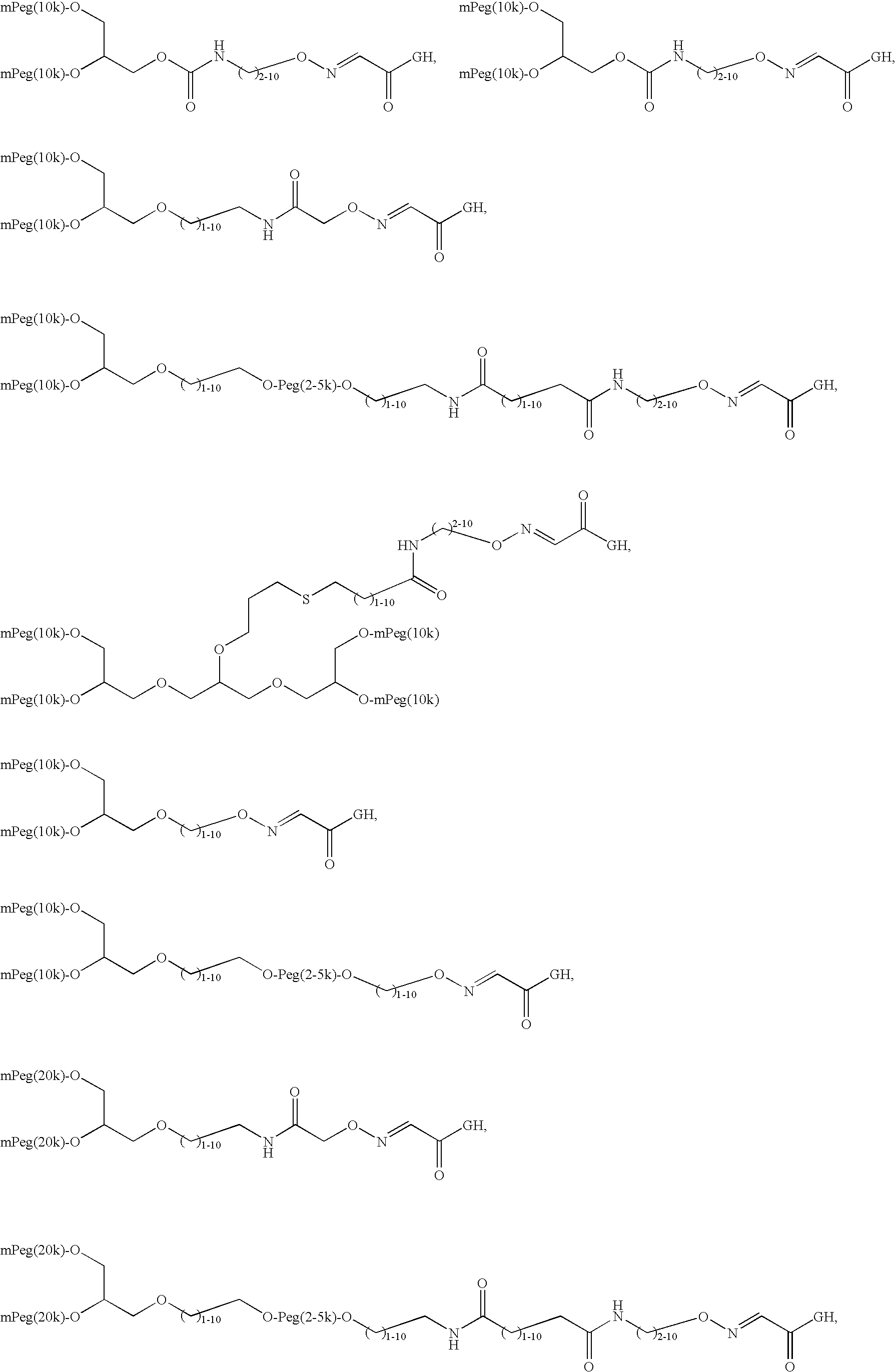

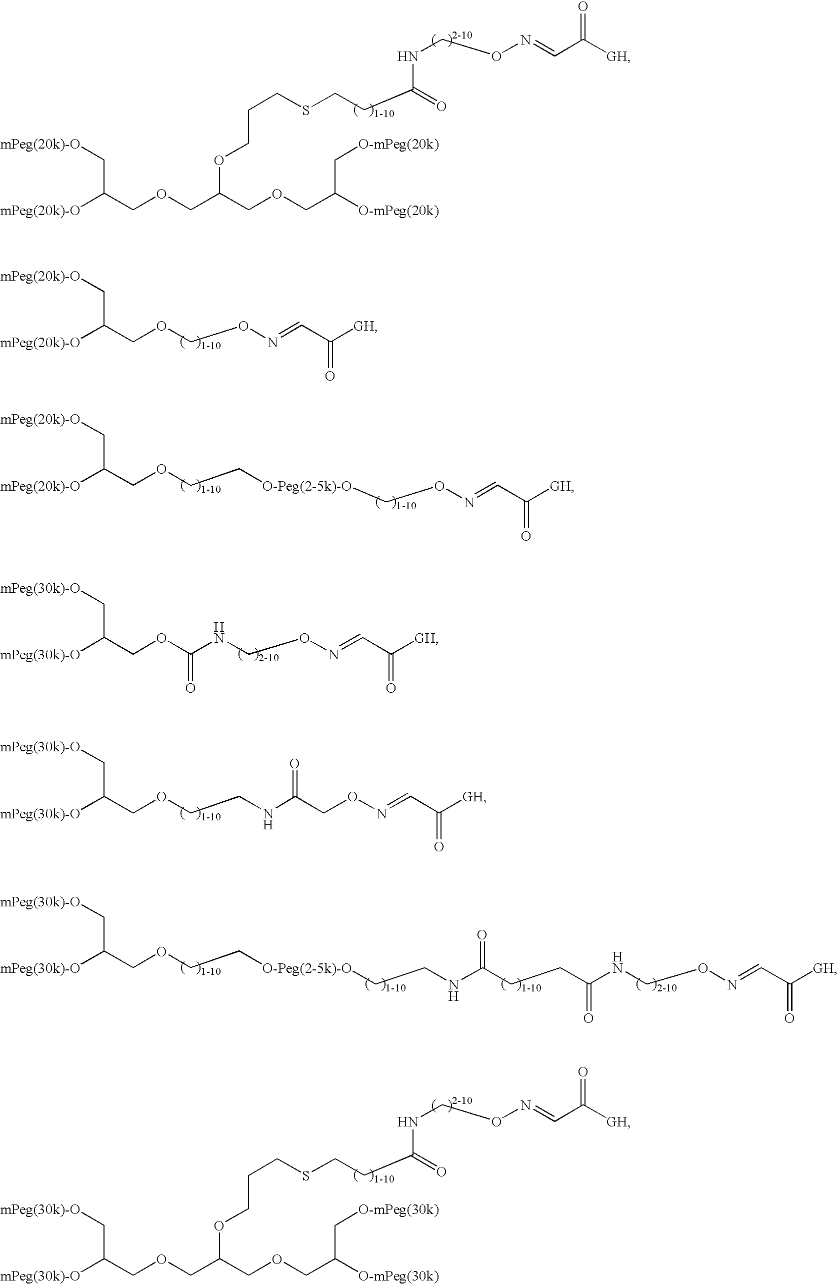

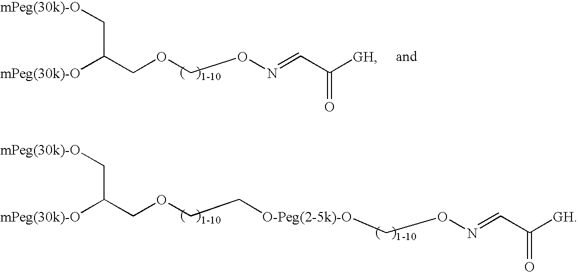

27. A method for preparing a compound according to claim 1, said method comprising the steps of
(a) reductive alkylation of the N-terminal amino group in a GH with a compound of the formula Q-Z-CH═O, wherein Q is a functional group which can be converted into an aldehyde or a ketone by treatment with a suitable reagent to obtain a compound of formula (II) 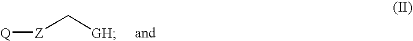
(b) conversion of the functional group Q in the compound of formula (II) into the aldehyde or ketone to obtain a compound of formula (III′) 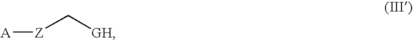
wherein A represents an aldehyde or ketone moiety; and
(c) condensation of the compound of formula III′ with a compound selected from R¹—O—NH₂, R²R³N—NH₂, R⁴NH(CH₂)₂₋₃SH or R⁵CH(NR⁶H)(CR⁷R⁸)₁₋₂SH, wherein R², R³, R⁴, R⁵, R⁶, R⁷ and R⁸ are as defined in claim 1.

28. The method according to claim 27, wherein Q is selected from 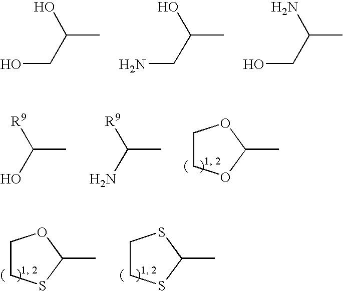
wherein R⁹ represents H, C₁₋₁₆alkyl, aryl, or heteroaryl, and the compound according to formula (III′) takes the form of formula (III)

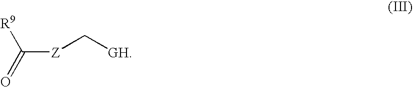

29. A method of preparing a compound according to claim 1, said method comprising the steps of
(a′) oxidating the N-terminally serine extended GH of formula (IV)
Ser-GH (IV)
to a glyoxylic acid derivative of formula (V)
O═CH—C(═O)-GH (V)
(b′) condensation of said derivative of formula V with a compound selected from R¹—O—NH₂, R²R³N—NH₂, R⁴NH(CH₂)₂₋₃SH or R⁵CH(NR⁶H)(CR⁷R⁸)₁₋₂SH, wherein R¹, R², R³, R⁴, R⁵, R⁶, R⁷ and R⁸ are as defined in claim 1.

30. The method according to claim 29, wherein said oxidation in step (a′) is effected by periodate.

31. A compound of formula (II) or (III) 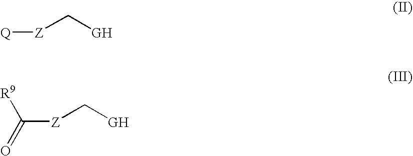
wherein Z represents a bond, alkylene, arylene, heteroarylene, or combinations thereof, and Q is a functional group which can be converted into an aldehyde or a ketone by treatment with a suitable reagent selected from 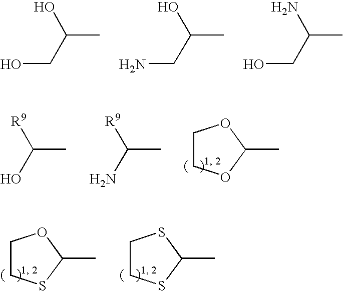
wherein R⁹ represents H, C₁₋₆alkyl, aryl, or heteroaryl.

32. The compound according to claim 31, wherein R⁹ represents hydrogen.

33. The compound according to claim 31, wherein Z represents —CH₂—O—(CH₂)₁₋₅ or —CH—O—(C₆H₄)—.

34. The compound according to claim 33, wherein Z represents —CH₂—O—(CH₂)₃, —CH₂—O—(CH₂)₄, —CH₂—O—(CH₂)₅, —CH₂—O-(1,4-C₆H₄)— or —CH₂—O-(1,3-C₆H₄)—.

35. The compound according claim 31, wherein GH represents the radical obtained by removal of the N-terminal amino group from hGH.

36. A compound according to formula IV,
Ser-GH (IV)
wherein GH represent a radical derived from a growth hormone compound by removal of one hydrogen atom from the N-terminal amino group, provided said compound is not Ser-hGH.

37. A compound according to formula V
O═CH—C(═O)-GH (V),
wherein GH represent a radical derived from a growth hormone compound by removal of one hydrogen atom from the N-terminal amino group.

38.-40. (canceled)

39. A nucleic acid construct comprising a nucleic acid sequence encoding a compound according to claim 36, provided that said construct does not encode Ser-hGH.

40. A vector comprising the construct according to claim 41.

41. A host cell comprising the vector according to claim 42.

42. (canceled)

43. A pharmaceutical composition comprising a compound according to claim 1.

44. A method of treating diseases benefiting from an increase in the level of circulating growth hormone, the method comprising the administration of a therapeutically effective amount of a compound according to claim 1 to a patient in need thereof.

45. The method according to claim 46, wherein said administration is performed every second day or with longer intervals.

46. (canceled)

49. A pharmaceutical composition comprising a compound according to claim 26.

50. A method of treating diseases benefiting from an increase in the level of circulating growth hormone, the method comprising the administration of a therapeutically effective amount of a compound according to claim 26 to a patient in need thereof.

51. The method according to claim 50, wherein said administration is performed every second day or with longer intervals.
